SEQUENCE LISTING (63 sequences)

SEQ ID NO: 1 - DNA, 1119 bp, Artificial Sequence
  Description of Artificial Sequence:  Synthetic Peptide
atgttagccaacagctcctcaaccaacagttctgttctcccgtgtcctgactaccgacctacccaccgcctgcacttggtggtctacagcttggtgctggctgccgggctccccctcaacgcgctagccctctgggtcttcctgcgcgcgctgcgcgtgcactcggtggtgagcgtgtacatgtgtaacctggcggccagcgacctgctcttcaccctctcgctgcccgttcgtctctcctactacgcactgcaccactggcccttccccgacctcctgtgccagacgacgggcgccatcttccagatgaacatgtacggcagctgcatcttcctgatgctcatcaacgtggaccgctacgccgccatcgtgcacccgctgcgactgcgccacctgcggcggccccgcgtggcgcggctgctctgcctgggcgtgtgggcgctcatcctggtgtttgccgtgcccgccgcccgcgtgcacaggccctcgcgttgccgctaccgggacctcgaggtgcgcctatgcttcgagagcttcagcgacgagctgtggaaaggcaggctgctgcccctcgtgctgctggccgaggcgctgggcttcctgctgcccctggcggcggtggtctactcgtcgggccgagtcttctggacgctggcgcgccccgacgccacgcagagccagcggcggcggaagaccgtgcgcctcctgctggctaacctcgtcatcttcctgctgtgcttcgtgccctacaacagcacgctggcggtctacgggctgctgcggagcaagctggtggcggccagcgtgcctgcccgcgatcgcgtgcgcggggtgctgatggtgatggtgctgctggccggcgccaactgcgtgctggacccgctggtgtactactttagcgccgagggcttccgcaacaccctgcgcggcctgggcactccgcaccgggccaggacctcggccaccaacgggacgcgggcggcgctcgcgcaatccgaaaggtccgccgtcaccaccgacgccaccaggccggatgccgccagtcaggggctgctccgaccctccgactcccactctctgtcttccttcacacagtgtccccaggattccgccctctga

SEQ ID NO: 2 - protein, 372 aa, Homo sapiens [sequence omitted]

SEQ ID NO: 3 - DNA, 3379 bp, Homo sapiens
gcgtccgaaaaaaaaagaaattcctttacatactacaacatgaatagatcttggaaacattatgctaagtgaaataaaccagacacaaaaggacaaatattgtatgattccactcatatgaggtatctagaataggcaaattcattgagacagaaagtagactagaaccagaagctgaatggggtgcggtgggtagtactgcttaatgactgcagagttgttgcttggttgatgaaaaagttctatttctggaaacagagagtggtgacggttaagcaacactgtcttggtcttttttgttgttgttgttgtttttgagacggactctcactctgtctcccaggccggagtgcgatggattagacctgctaggggagcacttggcaaaactcaacccacagggccttcccctgcctagcaagactgtgctgtcaaatttattcacatgtggctctggtcaagactagcatgcaatcagcctatgagggcattattatattattattcccattttacagatgaagaaactgagaagtcaaaccattaagctgaacccagtttgctttgaccacaaatccagccctcacaggcgcagtgatgcatgtgatgcgtaaggctgggatgttgttctgtatttgggagttttgtttgcttgtttgcttgtctgacatggagtctcactctgtcacccaggctggagtgcagtggcgtgatctcggctcactgcaacctccgcctcccgggttcaaggactctcctgctgcagcctcccatgtactcaaagagtttgacctttattctttggataatgaggagctagcctagcacctggtccaaggaggtgctccataagaccacctattgatttgtgcttattatctgtctccctccaatggaatgtaaaggaggtgggggcaaagactttttgctttgttccctgctgtgaacatgcctggaactttctatgagctcagtaagcaaggaaagaaggaaggaagagatcttgagatagtaacagcaacctaagcgttttacacacgtcatcttaatctccaaacctcatgaattctctctctctctctctcattttttgagacagagtctcgctctgtcacccaggctggagtgcagtggcgtgatctcgactcattgcaacctctgcctcctggattcaatcaattctcatgccttagcctactgaggagctgggattacaagtgcacgccaccatacccggctaatctttgtatttttagtagaggcaagattttgtcatgttggccaggttggtcttcaactcctggcctcaagtaatccacccacatcagcctcccaaagtgctgagatcacaggcatgaggtaccatgcagccgcctttttttttttttttgagatggagtctcgttttgttacccaggctggagggcagtggtacgatgtcagctcactgcaacctccgcctcctgggttcaagtgattctcctgtgtgagcctcctgagtagctgggactacaggtgcatgccaccacatctggctaatttttgtatttttagtagagacagggttttgccaggttggccaggctgatctcgaactcctgacctcaggtgatctgcccgcctcagtctcccaaagtgctggattacaggtgtgggccactacgccggccctggccctctttctttcttttttgagatgggctcactctgtcacccaggcaggagtgcagtggtgggcttgaggctcactgcactgcagcctccacctccctggagtcaagtgattctctcacctcagcctcacaagtagctgggactacgggcatgtgccacaatgcctggctaattttttaattttttaatattttttattttatttttttttgagacagagtcttgctctgtcacccaggccggagtgcaatggtgtgatctcggctcactgcaacctctgctcaagcaattctccctgccttagcctcctgagtagctgggattacaggcgcctgccaccacgcccggctaatttttttttttttttagtagagacaggattttgccatgttggccaggatggtctcaacctcctgacctcaggtgatccgcccacctcagcctcccaaagtgctcggattacagatgtgagccaccacgcccagccttatttttattttttattttattttatttatttattttgagatggagtttcactcttgttgcccaggctggagtgcaatggcgcgatcttggctcactgcaaactccaccccccaggttcaagcaattctcctgtctcagccccctgagtagctgggattacaggcgcccgcccctatgccaggctaatttttggtattttttttagtagagatggggtttcaccatgttggccaagctggtctcgaactcctgacctcaggtgatccacctgcctcggcctcccaaagtgctgggattacaggcgtgagccaccgcgcctggctatttttattttttgagacagagtttcacttttgttgtccaggctggagtgcaatggcacagtctcagctcactgcaacctctgcctcctggtttcaagcgattctcctgtctcagcctcccgagtagctgggattacaggcgtgcaccaccacgcccagctaatttttgtatttttagtagagatggggtttcaccatattggacaggttggtctcgaactcctgacctcaggtaatccacccgcctcggcctcccaaaatgctgggattacaggtgtgagccactgcacctggccctgtatttttttgtagagatggggtttcgccgtgttgcccaggctggtccccaactcctaggttcaagcaattggtctgccttggcctcccaaagtgccaggattacaggtgtaagccattgcacccagccaagattaatttttttgaagtcacacaactaggcaagttagcaaaaccaagatttaaacctaggcatccgagtccctgccttcaaacctgggtgtttaacactatactatatagtcctgccgtaggaacctattctagcccaatggcagacttgaggctgagaaaagattcagaaggcctgccagtggagctaaacatttgtgtgtgcagccctgtctctgtataacttccggcttgccttcctattccaggtctctgctgctgatgaagctgtgaccaaacgcacccaacccttggcagccatctgtccctgcagccatagcccacattcccatgacctccctctgcttgttttgggaccatgtctgtacagcctctaggccccagccccggaggtgaatgccatgccatgattctggtgtgctccatggcatccccagcctagctcccaatcccactttggcacg

SEQ ID NO: 4 - DNA, 1302 bp, Homo sapiens
acacacatgccattgcgctgtccgtgcccgactcccaacgcctctcgttctgggaggcttacagggtgtacacacaagaaggtgggctgggcacttggacctttgggtggcaattccagcttagcaacgcagaagagtacaaagtgtggaagccagggcccagggaaggcagtgctgctggaaatggcttctttaaactgtgagcacgcagagcaccccttctccagcggtgggaagtgatgcagagagcccacccgtgcagagggcagaagaggacgaaatgcctttgggtgggcagggcattaaactgctaaaagctggttagatggaacagaaaatgggcattctggatctaaaccgccacaggggcctgagagctgaagagcaccaggtttggtggacaaagctactgagatgcctgttcatctgctgacttctgtctaggctcatggatgccaccccctttcatttcggcctaggcttcccctgctcaccactgaggcctaatacaagagttcctatggacagaactacattctttctcgcatagtgacttgtgacaatttagacttggcatccagcatgggatagttggggcaaggcaaaactaacttagagtttccccctcaacaacatccaagtccaaaccctttttaggttatcctttcttccatcacatccccttttccaggcctcctccattttaggtccttaatattctttctttttctctctctctcgtttctctcttctctctcctctcctctctcttctcctcttctctctctctccctctctcctttgtccagagtaaggataaaattctttctactaaagcactggttctcaaactttttggtctcagaccccactcttagaaattgaggatctcaaagagctttgcttatattttgttcttttgatacttaccatactagaaattaaagcgaatacatttttaaaataaatacacatgcacacattacattagccatgggagcaataatgtcaccacacacacttcatgaagcctctggaaaactctacagtatacttgtgagagaatgagagtgaaagggacaaataacatctgtgtagcagtattatgaaaatagcttgacctcgtggacttcctcagagggttggtccctggatcacactttgagaaccatacttgtcctgaagtattggagttcatgtctaacttcttcccagggcattatgtacagtgctttttattactgtggggagagggcagtgctaaataaattaatcactactgataaaaaaaaaaaaaaaaag

SEQ ID NO: 5 - DNA, 80 bp, Artificial Sequence
  Description of Artificial Sequence: synthetic 5′ biotinylated oligonucleotide
ccaagctgtagaccaccaagtgcaggcggtgggtaggtcggtagtcaggacacgggagaacagaactgttggttgaggag

SEQ ID NO: 6 - DNA, 20 bp, Artificial Sequence
  Description of Artificial Sequence: synthetic HGPRBMY3 5′ primer
agcccaatggcagacttgag

SEQ ID NO: 7 - DNA, 20 bp, Artificial Sequence
  Description of Artificial Sequence:  Synthetic HGPRBMY3 3′ primer
ggtgcgtttggtcacagctt

SEQ ID NO: 8 - protein, 365 aa, Homo sapiens
MGNITADNSSMSCTIDHTIHQTLAPVVYVTVLVVGFPANCLSLYFGYLQIKARNELGVYLCNLTVADLFYICSLPFWLQYVLQHDNWSHGDLSCQVCGILLYENIYISVGFLCCISVDRYLAVAHPFRFHQFRTLKAAVGVSVVIWAKELLTSIYFLMHEEVIEDENQHRVCFEHYPIQAWQRAINYYRFLVGFLFPICLLLASYQGILRAVRRSHGTQKSRKDQIQRLVLSTVVIFLACFLPYHVLLLVRSVWEASCDFAKGVFNAYHFSLLLTSFNCVADPVLYCFVSETTHRDLARLRGACLAFLTCSRTGRAREAYPLGAPEASGKSGAQGEEPELLTKLHPAFQTPNSPGSGGFPTGRLA

SEQ ID NO: 9 - protein, 361 aa, HUMAN
MGNITADNTSMNCDIDHTIHQTLAPVVYVMVLVVGFPANCLSLYYGYLQIKARNELGVYLCNLTVADLFYICSLPFWLQYVLQHDHWSHDDLSCQVCGILLYENIYISVGFLCCISIDRYLAVAHPFRFHQFRTLKAAMGVSALIWVKELLTSIYFLMHEEVVEDADRHRVCFEHYPLEPRQRGINYYRFLVGFLFPICLLLASYRGILRAVRRSHGTQKSRKDQIQRLVLSTVVIFLACFLPYHVLLLVRSLWESSCDFAKGIFNAYHFSLLLTSFNCVADPVLYCFVSETTHRDLARLRGACLAFLTCARTGRAREAYPLGAPEASGKSEDPEVLTRLHPAFQTPHPPGMGGSPAGGLS

SEQ ID NO: 10 - protein, 370 aa, HUMAN [sequence omitted]

SEQ ID NO: 11 - protein, 370 aa, Homo sapiens
MGDRRFIDFQFQDSNSSLRPRLGNATANNTCIVDDSFKYNLNGAVYSVVFILGLITNSVSLFVFCFRMKMRSETAIFITNLAVSDLLFVCTLPFKIFYNFNRHWPFGDTLCKISGTAFLTNIYGSMLFLTCISVDRFLAIVYPFRSRTIRTRRNSAIVCAGVWILVLSGGISASLFSTTNVNNATTTCFEGFSKRVWKTYLSKITIFIEVVGFIIPLILNVSCSSVVLRTLRKPATLSQIGTNKKKVLKMITVHMAVFVVCFVPYNSVLFLYALVRSQAITNCFLERFAKIMYPITLCLATLNCCFDPFIYYFTLESFQKSFYINAHIRMESLFKTETPLTTKPSLPAIQEEVSDQTTNNGGELMLESTF

SEQ ID NO: 12 - protein, 308 aa, CHICKEN
MVSSNCSTEDSFKYTLYGCVFSMVFVLGLIANCVAIYIFTFTLKVRNETTTYMLNLAISDLLFVFTLPFRIYYFVVRNWPFGDVLCKISVTLFYTNMYGSILFLTCISVDRFLAIVHPFRSKTLRTKRNARIVCVAVWITVLAGSTPASFFQSTNRQNNTEQRTCFENFPESTWKTYLSRIVIFIEIVGFFIPLILNVTCSTMVLRTLNKPLTLSRNKLSKKKVLKMIFVHLVIFCFCFVPYNITLILYSLMRTQTWINCSVVTAVRTMYPVTLCIAVSNCCFDPIVYYFTSDTNSELDKKQQVHQNT

SEQ ID NO: 13 - protein, 344 aa, Homo sapiens
MVSVNSSHCFYNDSFKYTLYGCMFSMVFVLGLVSNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 14 - protein, 339 aa, Homo sapiens
MNGLEVAPPGLITNFSLATAEQCGQETPLENMLFASFYLLDFILALVGNTLALWLFIRDHKSGTPANVFLMHLAVADLSCVLVLPTRLVYHFSGNHWPFGEIACRLTGFLFYLNMYASIYFLTCISADRFLAIVHPVKSLKLRRPLYAHLACAFLWVVVAVAMAPLLVSPQTVQTNHTVVCLQLYREKASHHALVSLAVAFTFPFITTVTCYLLIIRSLRQGLRVEKRLKTKAVRMIAIVLAIFLVCFVPYHVNRSVYVLHYRSHGASCATQRILALANRITSCLTSLNGALDPIMYFFVAEKFRHALCNLLCGKRLKGPPPSFEGKTNESSLSAKSEL

SEQ ID NO: 15 - protein, 361 aa, RAT
MTSAESLLFTSLGPSPSSGDGDCRFNEEFKFILLPMSYAVVFVLGLALNAPTLWLFLFRLRPWDATATYMFHLALSDTLYVLSLPTLVYYYAARNHWPFGTGLCKFVRFLFYWNLYCSVLFLTCISVHRYLGICHPLRAIRWGRPRFASLLCLGVWLVVAGCLVPNLFFVTTNANGTTILCHDTTLPEEFDHYVYFSSAVMVLLFGLPFLITLVCYGLMARRLYRPLPGAGQSSSRLRSLRTIAVVLTVFAVCFVPFHITRTIYYQARLLQADCHVLNIVNVVYKVTRPLASANSCLDPVLYLFTGDKYRNQLQQLCRGSKPKPRTAASSLALVTLHEESISRWADTHQDSTFSAYEGDRL

SEQ ID NO: 16 - protein, 388 aa, Homo sapiens [sequence omitted]

SEQ ID NO: 17 - protein, 23 aa, Artificial Sequence
  Description of Artificial Sequence: Synthetic peptide
MLANSSSTNSSVLPCPDYRPTHR

SEQ ID NO: 18 - protein, 10 aa, Artificial Sequence
  Description of Artificial Sequence: Synthetic Peptide
RALRVHSVVS

SEQ ID NO: 19 - protein, 14 aa, Artificial Sequence
  Description of Artificial Sequence:  Synthetic Peptide
HHWPFPDLLCQTTG

SEQ ID NO: 20 - protein, 19 aa, Artificial Sequence
  Description of Artificial Sequence:  Synthetic Peptide
DRYAAIVHPLRLRHLRRPR

SEQ ID NO: 21 - protein, 35 aa, Artificial Sequence
  Description of Artificial Sequence:  Synthetic Peptide
AARVHRPSRCRYRDLEVRLCFESFSDELWKGRLLP

SEQ ID NO: 22 - protein, 23 aa, Artificial Sequence
  Description of Artificial Sequence:  Synthetic Peptide
RVFWTLARPDATQSQRRRKTVRL

SEQ ID NO: 23 - protein, 21 aa, Artificial Sequence
  Description of Artificial Sequence:  Synthetic Peptide
VYGLLRSKLVAASVPARDRVR

SEQ ID NO: 24 - protein, 72 aa, Artificial Sequence
  Description of Artificial Sequence:  Synthetic Peptide
AEGFRNTLRGLGTPHRARTSATNGTRAALAQSERSAVTTDATRTDAASQGLLRPSDSHSLSSFTQCPQDSAL

SEQ ID NO: 25 - protein, 19 aa, Artificial Sequence
  Description of Artificial Sequence:  Synthetic Peptide
TLARPDATQSQRRRKTVRL

SEQ ID NO: 26 - protein, 16 aa, Artificial Sequence
  Description of Artificial Sequence:  Synthetic Peptide
AQSERSAVTTDATRPD

SEQ ID NO: 27 - protein, 15 aa, Artificial Sequence
  Description of Artificial Sequence:  Synthetic Peptide
PAARVHRPSRCRYRD

SEQ ID NO: 28 - DNA, 23 bp, Artificial Sequence
  Description of Artificial Sequence: Synthetic GPCR19 forward primer
cctggcttccacactttgtactc

SEQ ID NO: 29 - DNA, 19 bp, Artificial Sequence
  Description of Artificial Sequence: Synthetic GPCR19 reverse primer
tcccaacgcctctcgttct

SEQ ID NO: 30 - DNA, 17 bp, Artificial Sequence
  Description of Artificial Sequence: Synthetic GAPDH-F3 forward primer
agccgagccacatcgct

SEQ ID NO: 31 - DNA, 19 bp, Artificial Sequence
  Description of Artificial Sequence: Synthetic GAPDH-R1 reverse primer
gtgaccaggcgcccaatac

SEQ ID NO: 32 - DNA, 28 bp, Artificial Sequence
  Description of Artificial Sequence:   Synthetic GAPDH-PVIC Taqman Probe
caaatccgttgactccgaccttcacctt

SEQ ID NO: 33 - DNA, 50 bp, Artificial Sequence
  Description of Artificial Sequence:  Synthetic HGPRBMY3 5′ primer
gtccccaagcttgcaccatgttagccaacagctcctcaaccaacagttct

SEQ ID NO: 34 - DNA, 72 bp, Artificial Sequence
  Description of Artificial Sequence: Synthetic HGPRBMY3 3′ Flag tag primer
gtccgcggatccctacttgtcgtcgtcgtccttgtagtccatgagggcggaatcctggggacactgtgtgaa

SEQ ID NO: 35 - DNA, 99 bp, Artificial Sequence
  Description of Artificial Sequence: Oligonucleotide 1 for library construction
  modified_base (25)..(84)
  n=a+g+c+t; n is located at nucleotide positions: 25,26,28,29,31,32,34,35,37,38,40,41,43,44,46,47,49 ,50,52,53,55,56,58,59,61,62,64,65,67,68,70,71,73,7 4,76,77,79,80,82,83
  k=c+g+t; k is located at nucleotide positions: 27,30,33,36,39,42,45,48,51,54,57,60,63,66,69,72,75 ,78,81,84
cgaagcgtaagggcccagccggccnnknnknnknnknnknnknnknnknnknnknnknnknnknnknnknnknnknnknnknnkccgggtccgggcggc

SEQ ID NO: 36 - DNA, 95 bp, Artificial Sequence
  Description of Artificial Sequence: Oligonucleotide 2 for library construction
  modified_base (21)..(80)
  n=a+g+c+t;  n is located at nucleotide positions: 22,23,25,26,28,29,31,32,34,35,37,38,40,41,43,44,46 ,47,49,50,52,53,55,56,58,59,61,62,64,65,67,68,70,7 1,73,74,76,77,79,80
  v= c+a+g; v is located at nucleotide positions: 21,24,27,30,33,36,39,42,45,48,51,54,57,60,63,66,69 ,72,75,78
aaaaggaaaaaagcggccgcvnnvnnvnnvnnvnnvnnvnnvnnvnnvnnvnnvnnvnnvnnvnnvnnvnnvnnvnnvnngccgcccggacccgg

SEQ ID NO: 37 - protein, 5 aa, Artificial Sequence
  Description of Artificial Sequence: Synthetic polypeptide
PGPGG

SEQ ID NO: 38 - protein, 15 aa, Artificial Sequence
  Description of Artificial Sequence: Synthetic polypeptide
GDFWYEACESSCAFW

SEQ ID NO: 39 - protein, 15 aa, Artificial Sequence
  Description of Artificial Sequence: Synthetic polypeptide
LEWGSDVFYDVYDCC

SEQ ID NO: 40 - protein, 15 aa, Artificial Sequence
  Description of Artificial Sequence: Synthetic polypeptide
CLRSGTGCAFQLYRF

SEQ ID NO: 41 - protein, 15 aa, Artificial Sequence
  Description of Artificial Sequence: Synthetic polypeptide
LFSSETFFDACCAFE

SEQ ID NO: 42 - protein, 14 aa, Artificial Sequence
  Description of Artificial Sequence: Synthetic polypeptide
RIDCCAKYFLRSCD

SEQ ID NO: 43 - DNA, 38 bp, Artificial Sequence
  Description of Artificial Sequence: Synthetic 5′ Primer
gcagcagcggccgccgcctgcacttggtggtctacagc

SEQ ID NO: 44 - DNA, 36 bp, Artificial Sequence
  Description of Artificial Sequence: Synthetic 3′ Primer
gcagcagtcgacgagggcggaatcctggggacactg

SEQ ID NO: 45 - DNA, 38 bp, Artificial Sequence
  Description of Artificial Sequence: Synthetic 5′ Primer
gcagcagcggccgcatgttagccaacagctcctcaacc

SEQ ID NO: 46 - DNA, 37 bp, Artificial Sequence
  Description of Artificial Sequence: Synthetic 3′ Primer
gcagcagtcgacggcgctaaagtagtacaccagcggg

SEQ ID NO: 47 - protein, 13 aa, Artificial Sequence
  Description of Artificial Sequence: Synthetic polypeptide
PDYRPTHRLHLVV

SEQ ID NO: 48 - protein, 13 aa, Artificial Sequence
  Description of Artificial Sequence: Synthetic polypeptide
AVVYSSGRVFWTL

SEQ ID NO: 49 - protein, 13 aa, Artificial Sequence
  Description of Artificial Sequence: Synthetic polypeptide
PDATQSQRRRKTV

SEQ ID NO: 50 - protein, 13 aa, Artificial Sequence
  Description of Artificial Sequence: Synthetic polypeptide
QRRRKTVRLLLAN

SEQ ID NO: 51 - protein, 13 aa, Artificial Sequence
  Description of Artificial Sequence: Synthetic polypeptide
EGFRNTLRGLGTP

SEQ ID NO: 52 - protein, 13 aa, Artificial Sequence
  Description of Artificial Sequence: Synthetic polypeptide
AALAQSERSAVTT

SEQ ID NO: 53 - protein, 14 aa, Artificial Sequence
  Description of Artificial Sequence: Synthetic polypeptide
RLCFESFSDELWKG

SEQ ID NO: 54 - protein, 14 aa, Artificial Sequence
  Description of Artificial Sequence: Synthetic polypeptide
VTTDATRPDAASQG

SEQ ID NO: 55 - protein, 14 aa, Artificial Sequence
  Description of Artificial Sequence: Synthetic polypeptide
TQSQRRRKTVRLLL

SEQ ID NO: 56 - protein, 12 aa, Artificial Sequence
  Description of Artificial Sequence: Synthetic polypeptide
MLANSSSTNSSV

SEQ ID NO: 57 - protein, 14 aa, Artificial Sequence
  Description of Artificial Sequence: Synthetic polypeptide
NSSSTNSSVLPCPD

SEQ ID NO: 58 - protein, 14 aa, Artificial Sequence
  Description of Artificial Sequence: Synthetic polypeptide
CFVPYNSTLAVYGL

SEQ ID NO: 59 - protein, 14 aa, Artificial Sequence
  Description of Artificial Sequence: Synthetic polypeptide
RTSATNGTRAALAQ

SEQ ID NO: 60 - protein, 17 aa, Artificial Sequence
  Description of Artificial Sequence: Synthetic polypeptide
LVLAAGLPLNALALAWV

SEQ ID NO: 61 - protein, 16 aa, Artificial Sequence
  Description of Artificial Sequence: Synthetic polypeptide
TLAVYGLLRSKLVAAS

SEQ ID NO: 62 - protein, 16 aa, Artificial Sequence
  Description of Artificial Sequence: Synthetic polypeptide
TSATNGTRAALAQSER

SEQ ID NO: 63 - protein, 27 aa, Artificial Sequence
  Description of Artificial Sequence: Synthetic polypeptide
QMNMYGSCIFLMLINVDRYAAIVHPLR

(19) United States
(12) Patent Application Publication
(10) Pub. No.: US 2003/0186360 A1
(43) Pub. Date: Oct. 2, 2003

(54) Novel human G-protein coupled receptor, HGPRBMY3, expressed highly in immune -and colon-related tissues

(76) Inventors: John N. Feder, Belle Mead, NJ (US); Gabe Mintier, Heightstown, NJ (US); Chandra S. Ramanathan, Wallingford, CT (US); Donald R. Hawken, Lawrenceville, NJ (US); Angela Cacace, Clinton, CT (US); Lauren Barber, Griswold, CT (US); Michael G. Kornacker, Princeton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/964,821

(22) Filed: Sep. 26, 2001

Related U.S. Application Data
(60) Provisional application No. 60/235,713, filed on Sep. 27, 2000.
(60) Provisional application No. 60/261,783, filed on Jan. 16, 2001.
(60) Provisional application No. 60/305,085, filed on Jul. 13, 2001.
(60) Provisional application No. 60/313,171, filed on Aug. 17, 2001.

(51) Int. Cl. C07K 14/705; C12Q 1/68; C07H 21/04; C12P 21/02; C12N 5/06

(52) U.S. Cl. 435/69.1; 435/320.1; 435/6; 435/325; 530/350; 536/23.2

(57) ABSTRACT

The present invention describes a newly discovered human G-protein coupled receptor and its encoding polynucleotide. Also described are expression vectors, host cells, agonists, antagonists, antisense molecules, and antibodies associated with the polynucleotide and/or polypeptide of the present invention. In addition, methods for treating, diagnosing, preventing, and screening for disorders associated with aberrant cell growth, immunological conditions, and diseases or disorders related to immune tissues, brain, and colon are illustrated.

### FIELD OF THE INVENTION

The present invention relates to the fields of pharmacogenomics, diagnostics and patient therapy. More specifically, the present invention relates to methods of diagnosing and/or treating diseases involving the Human G-Protein Coupled Receptor, HGPRBMY3.

### BACKGROUND OF THE INVENTION

It is well established that many medically significant biological processes are mediated by proteins participating in signal transduction pathways that involve G-proteins and/or second messengers, e.g., cAMP (Lefkowitz, Nature, 351:353-354 (1991)). Herein these proteins are referred to as proteins participating in pathways with G-proteins or PPG proteins. Some examples of these proteins include the GPC receptors, such as those for adrenergic agents and dopamine (Kobilka, B. K., et al., PNAS, 84:46-50 (1987); Kobilka, B. K., et al., Science, 238:650-656 (1987); Bunzow, J. R., et al., Nature, 336:783-787 (1988)), G-proteins themselves, effector proteins, e.g., phospholipase C, adenylate cyclase, and phosphodiesterase, and actuator proteins, e.g., protein kinase A and protein kinase C (Simon, M. I., et al., Science, 252:802-8 (1991)).

For example, in one form of signal transduction, the effect of hormone binding is activation of an enzyme, adenylate cyclase, inside the cell. Enzyme activation by hormones is dependent on the presence of the nucleotide GTP, and GTP also influences hormone binding. A G-protein connects the hormone receptors to adenylate cyclase. G-protein was shown to exchange GTP for bound GDP when activated by hormone receptors. The GTP-carrying form then binds to an activated adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of G-protein coupled receptors has been characterized as having seven putative transmembrane domains. The domains are believed to represent transmembrane α-helices connected by extracellular or cytoplasmic loops. G-protein coupled receptors include a wide range of biologically active receptors, such as hormone, viral, growth factor and neuroreceptors.

G-protein coupled receptors have been characterized as including these seven conserved hydrophobic stretches of about 20 to 30 amino acids, connecting at least eight divergent hydrophilic loops. The G-protein family of coupled receptors includes dopamine receptors, which bind to neuroleptic drugs, used for treating psychotic and neurological disorders. Other examples of members of this family include calcitonin, adrenergic, endothelin, cAMP, adenosine, muscarinic, acetylcholine, serotonin, histamine, thrombin, kinin, follicle stimulating hormone, opsins, endothelial differentiation gene-1 receptor, rhodopsins, odorant, cytomegalovirus receptors, etc.

Most G-protein coupled receptors have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or famesylation) of cysteine residues can influence signal transduction of some G-protein coupled receptors. Most G-protein coupled receptors contain potential phosphorylation sites within the third cytoplasmic loop and/or the carboxyl terminus. For several G-protein coupled receptors, such as the β-adrenoreceptor, phosphorylation by protein kinase A and/or specific receptor kinases mediates receptor desensitization.

For some receptors, the ligand binding sites of G-protein coupled receptors are believed to comprise a hydrophilic socket formed by several G-protein coupled receptors transmembrane domains, which socket is surrounded by hydrophobic residues of the G-protein coupled receptors. The hydrophilic side of each G-protein coupled receptor transmembrane helix is postulated to face inward and form the polar ligand-binding site. TM3 has been implicated in several G-protein coupled receptors as having a ligand-binding site, such as including the TM3 aspartate residue. Additionally, TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 10:317-331(1989)). Different G-protein β-subunits preferentially stimulate particular effectors to modulate various biological functions in a cell. Phosphorylation of cytoplasmic residues of G-protein coupled receptors have been identified as an important mechanism for the regulation of G-protein coupling of some G-protein coupled receptors. G-protein coupled receptors are found in numerous sites within a mammalian host.

G-protein coupled receptors (GPCRs) are one of the largest receptor superfamilies known. These receptors are biologically important and malfunction of these receptors results in diseases such as Alzheimer's, Parkinson, diabetes, dwarfism, color blindness, retinal pigmentosa and asthma. GPCRs are also involved in depression, schizophrenia, sleeplessness, hypertension, anxiety, stress, renal failure and in several other cardiovascular, metabolic, neural, oncology and immune disorders (F. Horn and G. Vriend, J. Mol. Med., 76: 464-468 (1998)). They have also been shown to play a role in HIV infection (Y. Feng et al., Science, 272: 872-877 (1996)). The structure of GPCRs consists of seven transmembrane helices that are connected by loops. The N-terminus is always extracellular and C-terminus is intracellular. GPCRs are involved in signal transduction. The signal is received at the extracellular N-terminus side. The signal can be an endogenous ligand, a chemical moiety or light. This signal is then transduced through the membrane to the cytosolic side where a heterotrimeric protein G-protein is activated which in turn elicits a response (F. Horn et al., Recept. and Chann., 5: 305-314 (1998)). Ligands, agonists and antagonists, for these GPCRs are used for therapeutic purposes.

The present invention provides a newly-discovered G-protein coupled receptor protein, which may be involved in cellular growth properties in immune-, testes-, colon-, breast-, and ovarian-related tissues based on its abundance found in said tissues. The present invention also relates to newly identified polynucleotides, polypeptides encoded by such polynucleotides, the use of such polynucleotides and polypeptides, as well as the production of such polynucleotides and polypeptides. More particularly, the polypeptides of the present invention are human 7-transmembrane receptors. The invention also relates to inhibiting the action of such polypeptides.

### SUMMARY OF THE INVENTION

The present invention provides a novel human member of the G-protein coupled receptor (GPCR) family (HGPRBMY3). Based on sequence homology, the protein HGPRBMY3 is a candidate GPCR. The HGPRBMY3 protein sequence has been predicted to contain seven transmembrane domains which is a characteristic structural feature of GPCRs. It is closely related to Purinergic receptor families based on sequence similarity. This orphan GPCR is expressed highly in immune-, testes-, and colon-, and breast-related tissues, while moderately expressed in ovarian tissues.

The present invention provides an isolated HGPRBMY3 polynucleotide as depicted in SEQ ID NO:1 (CDS: 1 to 1116).

The present invention also provides the HGPRBMY3 polypeptide (MW: 41.3 Kd), encoded by the polynucleotide of SEQ ID NO:1 and having the amino acid sequence of SEQ ID NO:2, or a functional or biologically active portion thereof.

The present invention further provides compositions comprising the HGPRBMY3 polynucleotide sequence, or a fragment thereof, or the encoded HGPRBMY3 polypeptide, or a fragment or portion thereof. Also provided by the present invention are pharmaceutical compositions comprising at least one HGPRBMY3 polypeptide, or a functional portion thereof, wherein the compositions further comprise a pharmaceutically acceptable carrier, excipient, or diluent.

The present invention provides a novel isolated and substantially purified polynucleotide that encodes the HGPRBMY3 GPCR homologue. In a particular aspect, the polynucleotide comprises the nucleotide sequence of SEQ ID NO: 1. The present invention also provides a polynucleotide sequence comprising the complement of SEQ ID NO: 1, or variants thereof. In addition, the present invention features polynucleotide sequences, which hybridize under moderately stringent or high stringency conditions to the polynucleotide sequence of SEQ ID NO: 1.

The present invention further provides a nucleic acid sequence encoding the HGPRBMY3 polypeptide and an antisense of the nucleic acid sequence, as well as oligonucleotides, fragments, or portions of the nucleic acid molecule or antisense molecule. Also provided are expression vectors and host cells comprising polynucleotides that encode the HGPRBMY3 polypeptide.

The present invention provides methods for producing a polypeptide comprising the amino acid sequence depicted in SEQ ID NO:2, or a fragment thereof, comprising the steps of a) cultivating a host cell containing an expression vector containing at least a functional fragment of the polynucleotide sequence encoding the HGPRBMY3 homologue according to this invention under conditions suitable for the expression of the polynucleotide; and b) recovering the polypeptide from the host cell.

Also provided are antibodies, and binding fragments thereof, which bind specifically to the HGPRBMY3 polypeptide, or an epitope thereof, for use as therapeutics and diagnostic agents.

The present invention also provides methods for screening for agents which modulate HGPRBMY3 polypeptide, e.g., agonists and antagonists, as well as modulators, e.g., agonists and antagonists, particularly those that are obtained from the screening methods described.

Also provided by the present invention is a substantially purified antagonist or inhibitor of the polypeptide of SEQ ID NO:2. In this regard, and by way of example, a purified antibody that binds to a polypeptide comprising the amino acid sequence of SEQ ID NO:2 is provided.

Substantially purified agonists of the polypeptide of SEQ ID NO:2 are further provided.

The present invention provides HGPRBMY3 nucleic acid sequences, polypeptide, peptides and antibodies for use in the diagnosis and/or screening of disorders or diseases associated with expression of the polynucleotide and its encoded polypeptide as described herein.

The present invention provides kits for screening and diagnosis of disorders associated with aberrant or uncontrolled cellular development and with the expression of the polynucleotide and its encoded polypeptide as described herein.

The present invention further provides methods for the treatment or prevention of cancers, immune disorders, testicular, colonic, breast, or ovarian diseases, or neurological disorders involving administering to an individual in need of treatment or prevention an effective amount of a purified antagonist of the HGPRBMY3 polypeptide. Due to its elevated expression in immune-, testes-, colon-, breast-, and ovarian-related tissues, the novel GPCR protein of the present invention is particularly useful in treating or preventing immunological, testicular, colonic, breast, and/or ovarian disorders, conditions, or diseases. Additionally, the GPCR protein of the invention may be used in the treatment or prevention of diseases, disorders, or conditions related to the breast or ovaries, as supported by the overexpression HGPRBMY3, GPCR19, or GPR92 in breast and ovarian cancer tissues.

The present invention also provides a method for detecting a polynucleotide that encodes the HGPRBMY3 polypeptide in a biological sample comprising the steps of: a) hybridizing the complement of the polynucleotide sequence encoding SEQ ID NO:2 to a nucleic acid material of a biological sample, thereby forming a hybridization complex; and b) detecting the hybridization complex, wherein the presence of the complex correlates with the presence of a polynucleotide encoding the HGPRBMY3 polypeptide in the biological sample. The nucleic acid material may be further amplified by the polymerase chain reaction prior to hybridization.

Further objects, features, and advantages of the present invention will be better understood upon a reading of the detailed description of the invention when considered in connection with the accompanying figures/drawings.

One aspect of the instant invention comprises methods and compositions to detect and diagnose alterations in the HGPRBMY3 sequence in tissues and cells as they relate to ligand response.

The present invention further provides compositions for diagnosing immunological disorders and response to HGPRBMY3 therapy in humans. In accordance with the invention, the compositions detect an alteration of the normal or wild type HGPRBMY3 sequence or its expression product in a patient sample of cells or tissue.

The present invention further provides diagnostic probes for diseases and a patient's response to therapy. The probe sequence comprises the HGPRBMY3 locus polymorphism. The probes can be constructed of nucleic acids or amino acids.

The present invention further provides antibodies that recognize and bind to the HGPRBMY3 protein. Such antibodies can be either polyclonal or monoclonal. Antibodies that bind to the HGPRBMY3 protein can be utilized in a variety of diagnostic and prognostic formats and therapeutic methods.

The present invention also provides diagnostic kits for the determination of the nucleotide sequence of human HGPRBMY3 alleles. The kits are based on amplification-based assays, nucleic acid probe assays, protein nucleic acid probe assays, antibody assays or any combination thereof.

The instant invention also provides methods for detecting genetic predisposition, susceptibility and response to therapy related to asthma and COPD. In accordance with the invention, the method comprises isolating a human sample, for example, blood or tissue from adults, children, embryos or fetuses, and detecting at least one alteration in the wild-type HGPRBMY3 sequence or its expression product from the sample, wherein the alterations are indicative of genetic predisposition, susceptibility or altered response to therapy related to immunological disorders.

In addition, methods for making determinations as to which drug to administer, dosages, duration of treatment and the like are provided.

### BRIEF DESCRIPTION OF THE FIGURES

FIG. 1 shows the full length nucleotide sequence of cDNA clone HGPRBMY3, a human G-protein coupled receptor (SEQ ID NO:1).

FIG. 2 shows the amino acid sequence (SEQ ID NO:2) from the conceptual translation of the full length HGPRBMY3 cDNA sequence.

FIGS. 3A-3B show the 5′ untranslated sequence of the orphan HGPRBMY3 (SEQ ID NO:3).

FIG. 4 shows the 3′ untranslated sequence of the orphan HGPRBMY3 (SEQ ID NO:4).

FIG. 5 shows the predicted transmembrane region of the HGPRBMY3 protein where the predicted transmembranes, bold-faced and underlined, correspond to the peaks with scores above 1000.

FIGS. 6A-6B show the multiple sequence alignment of the translated sequence of the orphan G-protein coupled receptor, HGPRBMY3, where the GCG pileup program was used to generate the alignment with purinergic and somatostatin receptor sequences. The blackened areas represent identical amino acids in more than half of the listed sequences and the grey highlighted areas represent similar amino acids. As shown in FIGS. 6A-6B, the sequences are aligned according to their amino acids, where: HGPRBMY3 (SEQ ID NO:2) is the translated full length HGPRBMY3 cDNA; GPR68 (SEQ ID NO:8) represents human ovarian cancer, GPCR 1; 046685 (SEQ ID NO:9) is the human orphan GPCR, BRGR1B; 015132 (SEQ ID NO:10) is the human form of a P2Y5-like purinergic receptor; P2Y9_HUMAN (SEQ ID NO:11) is the human form of the P2Y9 purinergic receptor; P2Y5_CHICK (SEQ ID NO:12) represents the chicken form of the purinergic receptor 5; P2Y5_HUMAN (SEQ ID NO: 13) is the human form of the purinergic receptor 5; GPRH_HUMAN (SEQ ID NO: 14) is the human form of the GPR17 receptor; 035811 (SEQ ID NO:15) is arat orphan GPCR; and SSR4_HUMAN (SEQ ID NO: 16) is the human form of the somatostatin receptor 4.

FIG. 7 shows the expression profiling of the novel human orphan GPCR, HGPRBMY3, as described in Example 3.

FIG. 8 shows the expression profiling of the novel human orphan GPCR, HGPRBMY3, as described in Table 1 and Example 5.

FIG. 9 shows the FACS profile for the untransfected CHO-NFAT/CRE cell line.

FIG. 10 shows the overexpression of HGPRBMY3 that constitutively couples through the NFAT/CRE Response Element.

FIG. 11 shows the FACS profile for the untransfected CHO-NFAT G alpha 15 cell line.

FIG. 12 shows the overexpression of HGPRBMY3 that constitutively couples through the NFAT response element via the promiscuous G protein, G alpha 15.

FIG. 13 shows the localization of expressed HGPRBMY3 to the cell surface.

FIG. 14 shows representative transfected CHO-NFAT/CRE cell lines with intermediate and high beta lactamase expression levels useful in screens to identify HGPRBMY3 agonists and/or antagonists.

### DETAILED DESCRIPTION OF THE PRESENT INVENTION

The present invention provides a novel isolated polynucleotide and encoded polypeptide, the expression of which is high in immune-, testes-, colon-, and breast-related tissues, and moderately high in ovarian tissues. This novel polypeptide is termed herein HGPRBMY3, an acronym for “Human G-Protein coupled Receptor BMY3”. HGPRBMY3 is also referred to as GPCR19 and GPR92.

Definitions

The HGPRBMY3 polypeptide (or protein) refers to the amino acid sequence of substantially purified HGPRBMY3, which may be obtained from any species, preferably mammalian, and more preferably, human, and from a variety of sources, including natural, synthetic, semi-synthetic, or recombinant. Functional fragments of the HGPRBMY3 polypeptide are also embraced by the present invention.

An “agonist” refers to a molecule which, when bound to the HGPRBMY3 polypeptide, or a functional fragment thereof, increases or prolongs the duration of the effect of the HGPRBMY3 polypeptide. Agonists may include proteins, nucleic acids, carbohydrates, or any other molecules that bind to and modulate the effect of HGPRBMY3 polypeptide. An antagonist refers to a molecule which, when bound to the HGPRBMY3 polypeptide, or a functional fragment thereof, decreases the amount or duration of the biological or immunological activity of HGPRBMY3 polypeptide. “Antagonists” may include proteins, nucleic acids, carbohydrates, antibodies, or any other molecules that decrease or reduce the effect of HGPRBMY3 polypeptide.

“Nucleic acid sequence”, as used herein, refers to an oligonucleotide, nucleotide, or poJynticleotide, and fragments or portions thereof, and to DNA or RNA of genomic or synthetic origin which may be single- or double-stranded, and represent the sense or anti-sense strand. By way of non-limiting example, fragments include nucleic acid sequences that are greater than 20-60 nucleotides in length, and preferably include fragments that are at least 70-100 nucleotides, or which are at least 1000 nucleotides or greater in length.

Similarly, “amino acid sequence” as used herein refers to an oligopeptide, peptide, polypeptide, or protein sequence, and fragments or portions thereof, and to naturally occurring or synthetic molecules. Amino acid sequence fragments are typically from about 5 to about 30, preferably from about 5 to about 15 amino acids in length and retain the biological activity or function of the HGPRBMY3 polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule. In addition, the phrases “HGPRBMY3 polypeptide” and “HGPRBMY3 protein” are used interchangeably herein to refer to the encoded product of the HGPRBMY3 nucleic acid sequence of the present invention.

A “variant” of the HGPRBMY3 polypeptide refers to an amino acid sequence that is altered by one or more amino acids. The variant may have “conservative” changes, wherein a substituted amino acid has similar structural or chemical properties, e.g., replacement of leucine with isoleucine. More rarely, a variant may have “non-conservative” changes, e.g., replacement of a glycine with a tryptophan. Minor variations may also include amino acid deletions or insertions, or both. Guidance in determining which amino acid residues may be substituted, inserted, or deleted without abolishing functional biological or immunological activity may be found using computer programs well known in the art, for example, DNASTAR software.

An “allele” or “allelic sequence” is an alternative form of the HGPRBMY3 nucleic acid sequence. Alleles may result from at least one mutation in the nucleic acid sequence and may yield altered mRNAs or polypeptides whose structure or function may or may not be altered. Any given gene, whether natural or recombinant, may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

“Altered” nucleic acid sequences encoding HGPRBMY3 polypeptide include nucleic acid sequences containing deletions, insertions and/or substitutions of different nucleotides resulting in a polynucleotide that encodes the same or a functionally equivalent HGPRBMY3 polypeptide. Altered nucleic acid sequences may further include polymorphisms of the polynucleotide encoding the HGPRBMY3 polypeptide; such polymorphisms may or may not be readily detectable using a particular oligonucleotide probe. The encoded protein may also contain deletions, insertions, or substitutions of amino acid residues, which produce a silent change and result in a functionally equivalent HGPRBMY3 protein. Deliberate amino acid substitutions may be made on the basis of similarity in polarity, charge, solubility, hydrophobicity, hydrophilicity, and/or the amphipathic nature of the residues, as long as the biological activity of HGPRBMY3 protein is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; and phenylalanine and tyrosine.

“Peptide nucleic acid” (PNA) refers to an antisense molecule or anti-gene agent which comprises an oligonucleotide (“oligo”) linked via an amide bond, similar to the peptide backbone of amino acid residues. PNAs typically comprise oligos of at least 5 nucleotides linked via amide bonds. PNAs may or may not terminate in positively charged amino acid residues to enhance binding affinities to DNA. Such amino acids include, for example, lysine and arginine, among others. These small molecules stop transcript elongation by binding to their complementary strand of nucleic acid (P. E. Nielsen et al., 1993, Anticancer Drug Des., 8:53-63). PNA may be pegylated to extend their lifespan in the cell where they preferentially bind to complementary single stranded DNA and RNA.

“Oligonucleotides” or “oligomers” refer to a nucleic acid sequence, preferably comprising contiguous nucleotides, of at least about 6 nucleotides to about 60 nucleotides, preferably at least about 8 to 10 nucleotides in length, more preferably at least about 12 nucleotides in length e.g., about 15 to 35 nucleotides, or about 15 to 25 nucleotides, or about 20 to 35 nucleotides, which can be typically used in PCR amplification assays, hybridization assays, or in microarrays. It will be understood that the term “oligonucleotide” is substantially equivalent to the terms primer, probe, or amplimer, as commonly defined in the art. It will also be appreciated by those skilled in the pertinent art that a longer oligonucleotide probe, or mixtures of probes, e.g., degenerate probes, can be used to detect longer, or more complex, nucleic acid sequences, for example, genomic DNA. In such cases, the probe may comprise at least 20-200 nucleotides, preferably, at least 30-100 nucleotides, more preferably, 50-100 nucleotides.

“Amplification” refers to the production of additional copies of a nucleic acid sequence and is generally carried out using polymerase chain reaction (PCR) technologies, which are well known and practiced in the art (see, D. W. Dieffenbach and G. S. Dveksler, 1995, PCR Primer, a Laboratory Manual, Cold Spring Harbor Press, Plainview, N.Y.).

“Microarray” is an array of distinct polynucleotides or oligonucleotides synthesized on a substrate, such as paper, nylon, or other type of membrane; filter; chip; glass slide; or any other type of suitable solid support.

The term “antisense” refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNA and may be produced by any method, including synthesis or transcription. Once introduced into a cell, the complementary nucleotides combine with natural sequences produced by the cell to form duplexes, which block either transcription or translation. The designation “negative” is sometimes used in reference to the antisense strand, and “positive” is sometimes used in reference to the sense strand.

The term “consensus” refers to the sequence that reflects the most common choice of base or amino acid at each position among a series of related DNA, RNA or protein sequences. Areas of particularly good agreement often represent conserved functional domains.

A “deletion” refers to a change in either nucleotide or amino acid sequence and results in the absence of one or more nucleotides or amino acid residues. By contrast, an insertion (also termed “addition”) refers to a change in a nucleotide or amino acid sequence that results in the addition of one or more nucleotides or amino acid residues, as compared with the naturally occurring molecule. A substitution refers to the replacement of one or more nucleotides or amino acids by different nucleotides or amino acids.

A “derivative” nucleic acid molecule refers to the chemical modification of a nucleic acid encoding, or complementary to, the encoded HGPRBMY3 polypeptide. Such modifications include, for example, replacement of hydrogen by an alkyl, acyl, or amino group. A nucleic acid derivative encodes a polypeptide, which retains the essential biological and/or functional characteristics of the natural molecule. A derivative polypeptide is one, which is modified by glycosylation, pegylation, or any similar process that retains the biological and/or functional or immunological activity of the polypeptide from which it is derived.

The term “biologically active”, i.e., functional, refers to a protein or polypeptide or fragment thereof having structural, regulatory, or biochemical functions of a naturally occurring molecule. Likewise, “immunologically active” refers to the capability of the natural, recombinant, or synthetic HGPRBMY3, or any oligopeptide thereof, to induce a specific immune response in appropriate animals or cells, for example, to generate antibodies, and to bind with specific antibodies.

The term “hybridization” refers to any process by which a strand of nucleic acid binds with a complementary strand through base pairing.

The term “hybridization complex” refers to a complex formed between two nucleic acid sequences by virtue of the formation of hydrogen bonds between complementary G and C bases and between complementary A and T bases. The hydrogen bonds may be further stabilized by base stacking interactions. The two complementary nucleic acid sequences hydrogen bond in an anti-parallel configuration. A hybridization complex may be formed in solution (e.g., Cot or Rot analysis), or between one nucleic acid sequence present in solution and another nucleic acid sequence immobilized on a solid support (e.g., membranes, filters, chips, pins, or glass slides, or any other appropriate substrate to which cells or their nucleic acids have been affixed).

The terms “stringency” or “stringent conditions” refer to the conditions for hybridization as defined by nucleic acid composition, salt and temperature. These conditions are well known in the art and may be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (e.g., formamide, dextran sulfate and/or polyethylene glycol) and reaction temperature (within a range of from about 5° C. below the melting temperature of the probe to about 20° C. to 25° C. below the melting temperature). One or more factors may be varied to generate conditions, either low or high stringency, that are different from but equivalent to the aforementioned conditions.

As will be understood by those of skill in the art, the stringency of hybridization may be altered in order to identify or detect identical or related polynucleotide sequences. As will be further appreciated by the skilled practitioner, the melting temperature, Tₘ, can be approximated by the formulas as known in the art, depending on a number of parameters, such as the length of the hybrid or probe in number of nucleotides, or hybridization buffer ingredients and conditions (see, for example, T. Maniatis et al., Molecular Cloning: A Laboratory Manual, Cold Spring Harbor Laboratory, Cold Spring Harbor, N.Y., 1982 and J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA”, John Wiley and Sons, Inc., 1994-1995, Suppls. 26, 29, 35 and 42; pp. 2.10.7-2.10.16; G. M. Wahl and S. L. Berger (1987; Methods Enzymol. 152:399-407); and A. R. Kimmel, 1987; Methods of Enzymol. 152:507-511). As a general guide, Tₘ decreases approximately 1° C.-1.5° C. with every 1% decrease in sequence homology. Also, in general, the stability of a hybrid is a function of sodium ion concentration and temperature. Typically, the hybridization reaction is initially performed under conditions of low stringency, followed by washes of varying, but higher stringency. Reference to hybridization stringency, e.g., high, moderate, or low stringency, typically relates to such washing conditions.

Thus, by way of non-limiting example, “high stringency” refers to conditions that permit hybridization of those nucleic acid sequences that form stable hybrids in 0.018M NaCl at about 65° C. (i.e., if a hybrid is not stable in 0.018M NaCl at about 65° C., it will not be stable under high stringency conditions). High stringency conditions can be provided, for instance, by hybridization in 50% formamide, 5× Denhardt's solution, 5× SSPE (saline sodium phosphate EDTA) (lx SSPE buffer comprises 0.15 M NaCl, 10 mM Na₂HPO₄, 1 mM EDTA), (or 1× SSC buffer containing 150 mM NaCl, 15 mM Na₃ citrate·2H₂O, pH 7.0), 0.2% SDS at about 42° C., followed by washing in 1× SSPE (or saline sodium citrate, SSC) and 0.1% SDS at a temperature of at least about 42° C., preferably about 55° C., more preferably about 65° C.

“Moderate stringency” refers, by non-limiting example, to conditions that permit hybridization in 50% formamide, 5× Denhardt's solution, 5× SSPE (or SSC), 0.2% SDS at 42° C. (to about 50° C.), followed by washing in 0.2× SSPE (or SSC) and 0.2% SDS at a temperature of at least about 42° C., preferably about 55° C., more preferably about 65° C.

“Low stringency” refers, by non-limiting example, to conditions that permit hybridization in 10% formamide, 5× Denhardt's solution, 6× SSPE (or SSC), 0.2% SDS at 42° C., followed by washing in 1× SSPE (or SSC) and 0.2% SDS at a temperature of about 45° C., preferably about 50° C.

For additional stringency conditions, see T. Maniatis et al., Molecular Cloning: A Laboratory Manual, Cold Spring Harbor Laboratory, Cold Spring Harbor, N.Y. (1982). It is to be understood that the low, moderate and high stringency hybridization/washing conditions may be varied using a variety of ingredients, buffers and temperatures well known to and practiced by the skilled artisan.

The terms “complementary” or “complementarity” refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands, as well as in the design and use of PNA molecules.

The term “homology” refers to a degree of complementarity. There may be partial homology or complete homology, wherein complete homology is equivalent to identity. A partially complementary sequence that at least partially inhibits an identical sequence from hybridizing to a target nucleic acid is referred to using the functional term “substantially homologous”. The inhibition of hybridization of the completely complementary sequence to the target sequence may be examined using a hybridization assay (e.g., Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. Nonetheless, conditions of low stringency do not permit non-specific binding; low stringency conditions require that the binding of two sequences to one another be a specific (i.e., selective) interaction. The absence of non-specific binding may be tested by the use of a second target sequence which lacks even a partial degree of complementarity (e.g., less than about 30% identity). In the absence of non-specific binding, the probe will not hybridize to the second non-complementary target sequence.

Those having skill in the art will know how to determine percent identity between or among sequences using, for example, algorithms such as those based on the CLUSTALW computer program (J. D. Thompson et al., 1994, Nucleic Acids Research, 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations.

A “composition” comprising a given polynucleotide sequence refers broadly to any composition containing the given polynucleotide sequence. The composition may comprise a dry formulation or an aqueous solution. Compositions comprising polynucleotide sequence (SEQ ID NO: 1) encoding HGPRBMY3 polypeptide (SEQ ID NO:2), or fragments thereof, may be employed as hybridization probes. The probes may be stored in freeze-dried form and may be in association with a stabilizing agent such as a carbohydrate. In hybridizations, the probe may be employed in an aqueous solution containing salts (e.g., NaCl), detergents or surfactants (e.g., SDS) and other components (e.g., Denhardt's solution, dry milk, salmon sperm DNA, and the like).

The term “substantially purified” refers to nucleic acid sequences or amino acid sequences that are removed from their natural environment, isolated or separated, and are at least 60% free, preferably 75% to 85% free, and most preferably 90% or greater free from other components with which they are naturally associated.

The term “sample”, or “biological sample”, is meant to be interpreted in its broadest sense. A biological sample suspected of containing nucleic acid encoding HGPRBMY3 protein, or fragments thereof, or HGPRBMY3 protein itself, may comprise a body fluid, an extract from cells or tissue, chromosomes isolated from a cell (e.g., a spread of metaphase chromosomes), organelle, or membrane isolated from a cell, a cell, nucleic acid such as genomic DNA (in solution or bound to a solid support such as for Southern analysis), RNA (in solution or bound to a solid support such as for Northern analysis), cDNA (in solution or bound to a solid support), a tissue, a tissue print and the like.

“Transformation” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells, which transiently express the inserted DNA or RNA for limited periods of time.

The term “mimetic” refers to a molecule, the structure of which is developed from knowledge of the structure of HGPRBMY3 protein, or portions thereof, and as such, is able to effect some or all of the actions of HGPRBMY3 protein.

The term “portion” with regard to a protein (as in “a portion of a given protein”) refers to fragments or segments of that protein. The fragments may range in size from four or five amino acid residues to the entire amino acid sequence minus one amino acid. Thus, a protein “comprising at least a portion of the amino acid sequence of SEQ ID NO:2” encompasses the full-length human HGPRBMY3 polypeptide, and fragments thereof.

The term “antibody” refers to intact molecules as well as fragments thereof, such as Fab, F(ab′)₂, Fv, which are capable of binding an epitopic or antigenic determinant. Antibodies that bind to HGPRBMY3 polypeptides can be prepared using intact polypeptides or fragments containing small peptides of interest or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (e.g, a mouse, a rat, or a rabbit).

The term “humanized” antibody refers to antibody molecules in which amino acids have been replaced in the non-antigen binding regions in order to more closely resemble a human antibody, while still retaining the original binding capability, e.g., as described in U.S. Pat. No. 5,585,089 to C. L. Queen et al.

The term “antigenic determinant” refers to that portion of a molecule that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to an antigenic determinants. An antigenic determinant may compete with the intact antigen (i.e., the immunogen used to elicit the immune response) for binding to an antibody.

The terms “specific binding” or “specifically binding” refer to the interaction between a protein or peptide and a binding molecule, such as an agonist, an antagonist, or an antibody. The interaction is dependent upon the presence of a particular structure (i.e., an antigenic determinant or epitope) of the protein that is recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

The term “correlates with expression of a polynucleotide” indicates that the detection of the presence of ribonucleic acid that is similar to SEQ ID NO: 1 by Northern analysis is indicative of the presence of mRNA encoding HGPRBMY3 polypeptide (SEQ ID NO:2) in a sample and thereby correlates with expression of the transcript from the polynucleotide encoding the protein.

An alteration in the polynucleotide of SEQ ID NO: 1 comprises any alteration in the sequence of the polynucleotides encoding HGPRBMY3 polypeptide, including deletions, insertions, and point mutations that may be detected using hybridization assays. Included within this definition is the detection of alterations to the genomic DNA sequence which encodes HGPRBMY3 polypeptide (e.g., by alterations in the pattern of restriction fragment length polymorphisms capable of hybridizing to SEQ ID NO: 1), the inability of a selected fragment of SEQ ID NO: 1 to hybridize to a sample of genomic DNA (e.g., using allele-specific oligonucleotide probes), and improper or unexpected hybridization, such as hybridization to a locus other than the normal chromosomal locus for the polynucleotide sequence encoding HGPRBMY3 polypeptide (e.g., using fluorescent in situ hybridization (FISH) to metaphase chromosome spreads).

### DESCRIPTION OF THE INVENTION

The present invention provides a novel human member of the G-protein coupled receptor (GPCR) family (HGPRBMY3). Based on sequence homology, the protein HGPRBMY3 is a novel human GPCR. This protein sequence has been predicted to contain seven transmembrane domains, which is a characteristic structural feature of GPCRs. It is closely related to purinergic receptor families based on sequence similarity. This orphan GPCR is expressed highly in immune-, testes-, colon-, and breast-related tissues, and moderately high in ovarian tissues. HGPRBMY3 polypeptides and polynucleotides are useful for diagnosing diseases related to over- or under-expression of HGPRBMY3 proteins by identifying mutations in the HGPRBMY3 gene using HGPRBMY3 probes, or determining HGPRBMY3 protein or mRNA expression levels. HGPRBMY3 polypeptides are also useful for screening compounds, which affect activity of the protein. The invention encompasses the polynucleotide encoding the HGPRBMY3 polypeptide and the use of the HGPRBMY3 polynucleotide or polypeptide, or composition in thereof, the screening, diagnosis, treatment, or prevention of disorders associated with aberrant or uncontrolled cellular growth and/or function, such as neoplastic diseases (e.g., cancers and tumors), with particular regard to diseases or disorders related to the immune system. More specifically, diseases that can be treated with HGPRBMY3 include HIV infections, pain, anorexia, intestinal bowel disorders, cancers, bulimia, asthma, Parkinson's disease, acute heart failure, hypotension, hypertension, urinary retention, osteoporosis, angina pectoris, myocardial infarction, ulcers, allergies, benign prostatic hypertrophy, psychotic, immune, metabolic, cardiovascular and neurological disorders. Additionally, HGPRBMY3 may be used in particular for the diagnosis, treatment, or prevention of diseases, disorders, or conditions of the testes, colon, breast, or ovaries, for example, but not limited to testicular, colon, breast, and ovarian cancer.

Nucleic acids encoding human HGPRBMY3 according to the present invention were first identified in Incyte CloneID:3356166 from a prostate tumor library through a computer search for amino acid sequence alignments (see Example 1).

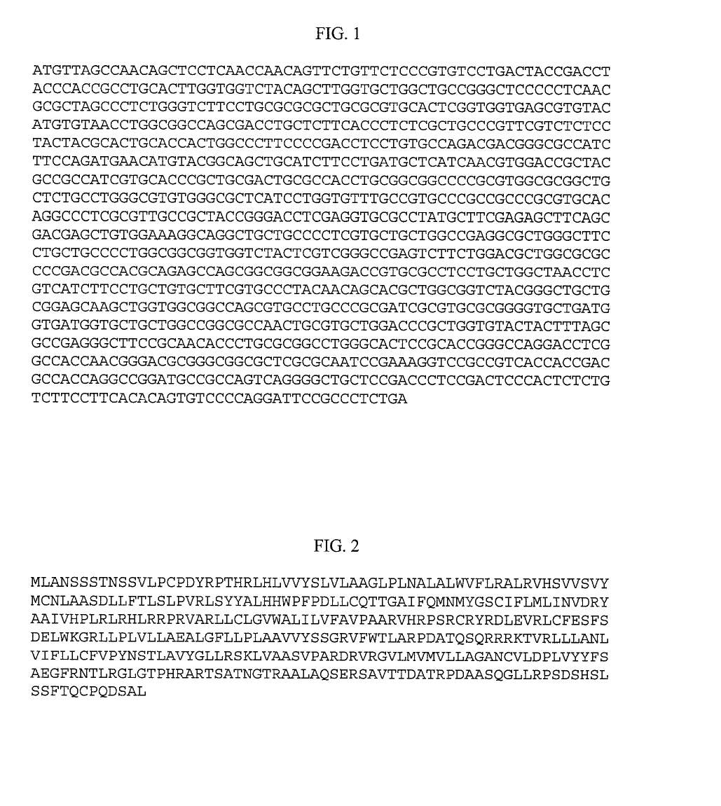

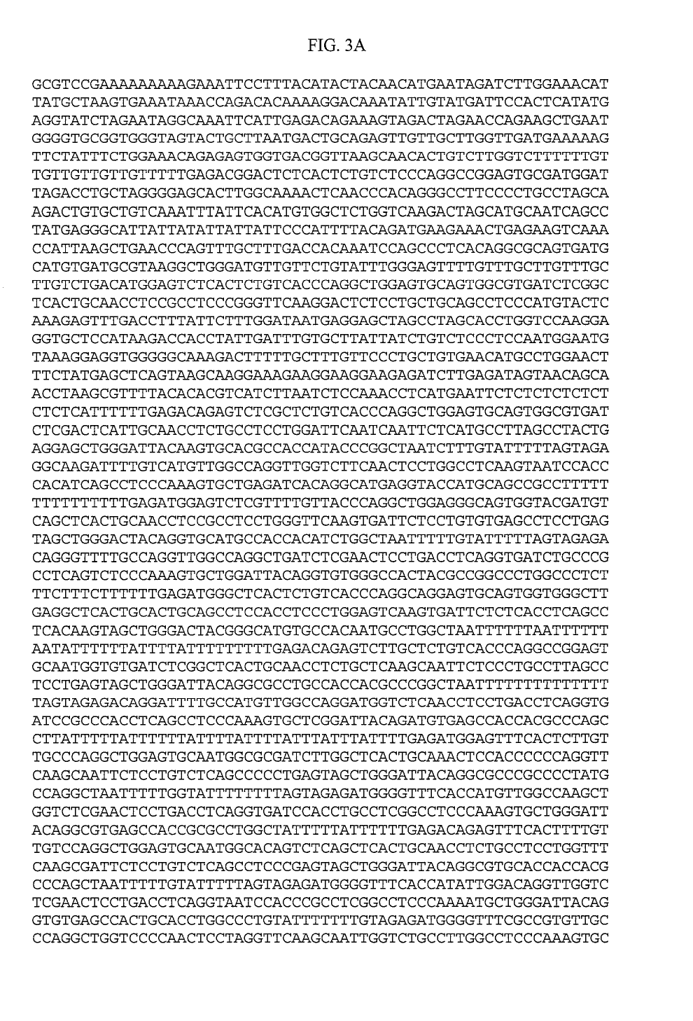

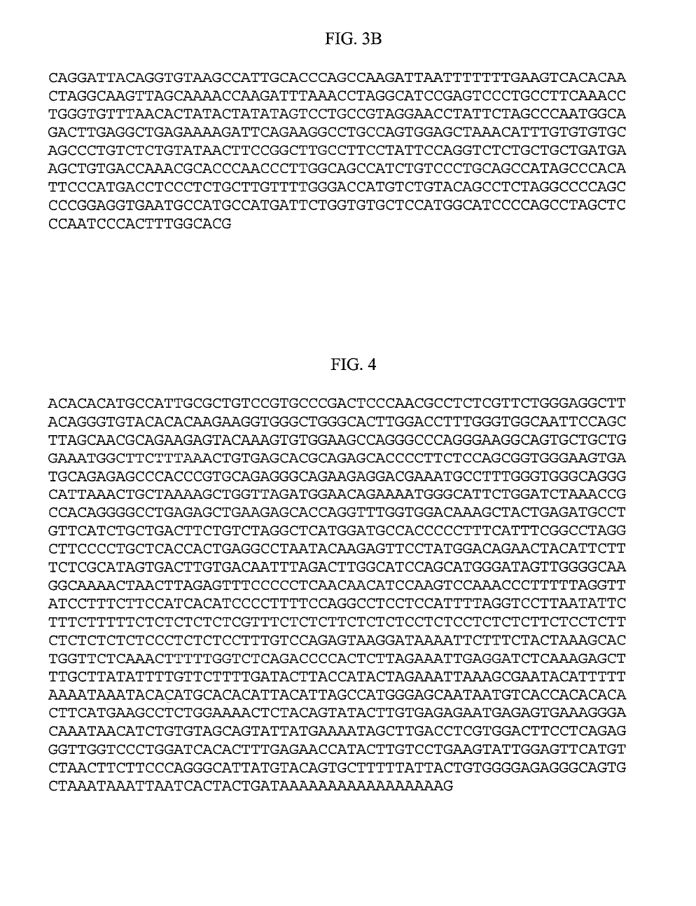

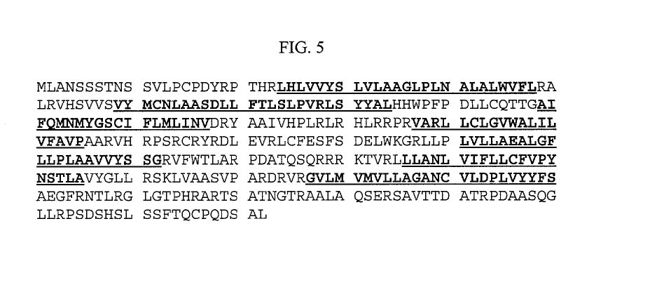

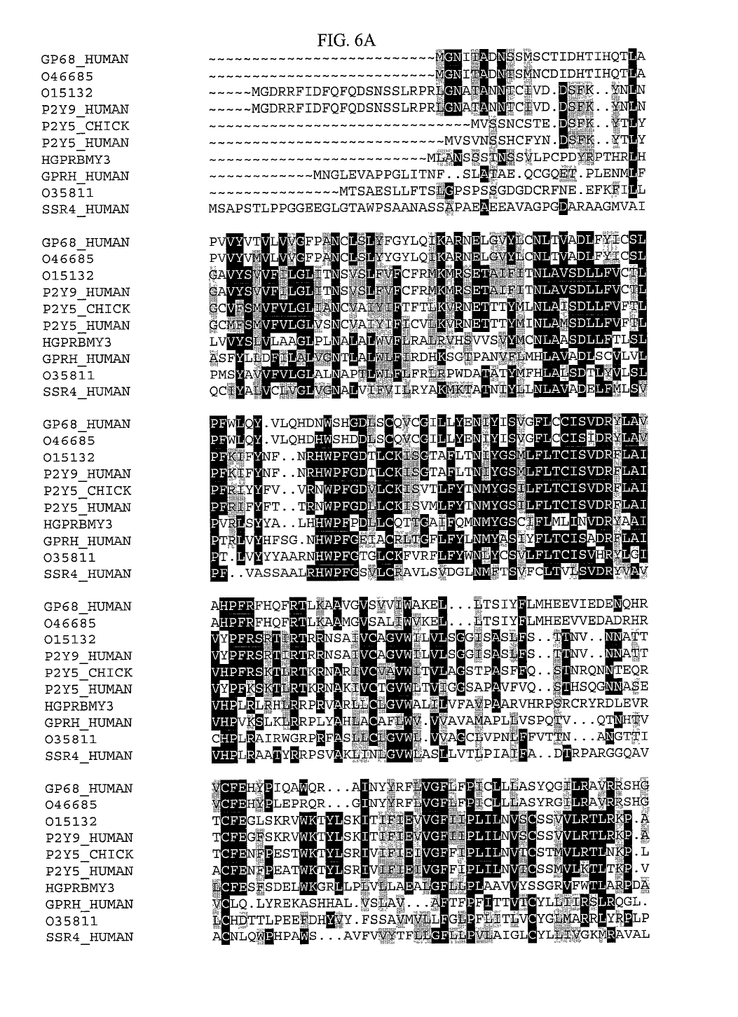

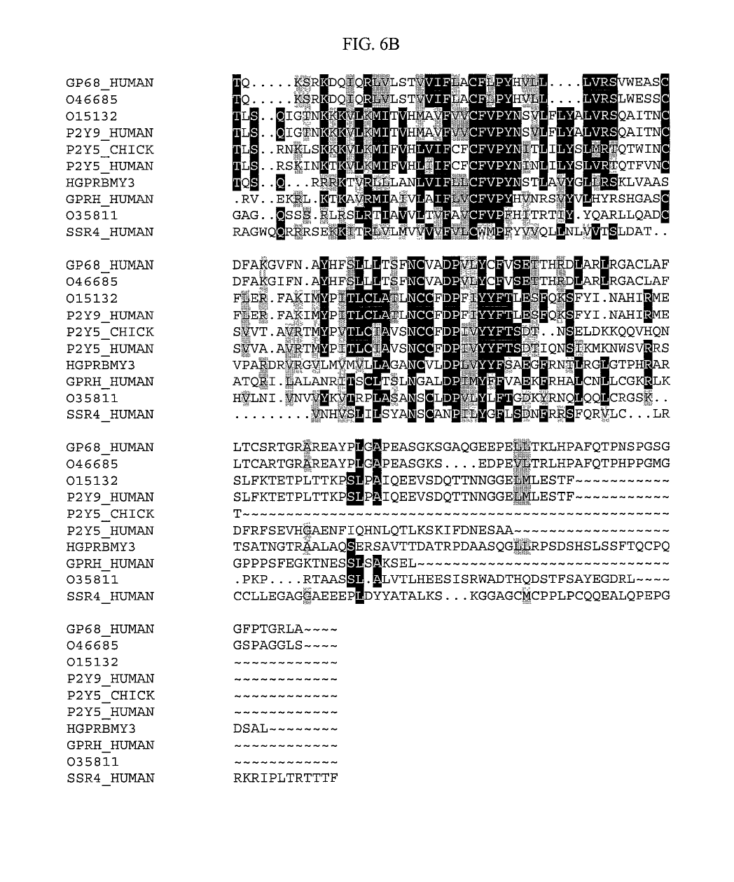

In one of its embodiments, the present invention encompasses a polypeptide comprising the amino acid sequence of SEQ ID NO:2 as shown in FIG. 1. The HGPRBMY3 polypeptide is 372 amino acids in length and shares amino acid sequence homology with the chicken P2Y purinergic receptor 5 (P2Y5_CHICK; Acc. No.:P32250). The HGPRBMY3 polypeptide (SEQ ID NO:2) shares 39% identity and 49.5% similarity with 284 amino acids of the chicken P2Y purinergic receptor 5, wherein “similar” amino acids are those which have the same/similar physical properties and in many cases, the function is conserved with similar residues. For example, amino acids Lysine and Arginine are similar; whereas residues such as Proline and Cysteine do not share any physical property and they are not considered similar. The HGPRBMY3 polypeptide shares 31.5% identity and 37.5% similarity with the human GP68 receptor (GP68_HUMAN); 32.1% identity and 41.4% similarity with the human GPCR (GPRH_HUMAN; Acc. No.:Q13304); 34.5% identity and 44.7% similarity with human P2Y5-like receptor (Acc. No.:015132); 35.3% identity and 40.8% similarity with rattus norvegicus G-protein coupled receptor (Acc. No.:035811); 34.2% identity and 39.9% similarity with bos taurus orphan G-protein-coupled receptor BRGR1B (Acc. No.:046685); 35.5% identity and 46.7% similarity with human P2Y5 (P2Y5_HUMAN; Acc. No.:P43657; 015133); 34.8% identity and 45% similarity with human P2Y purinoceptor 9 (P2Y9; P2Y9_HUMAN; Acc. No.:Q99677); and 31.4% identity and 38% similarity with human somatostatin receptor type 4 (SS4R; SSR4_HUMAN; Acc. No.:P31391).

Variants of the HGPRBMY3 polypeptide are also encompassed by the present invention. A preferred HGPRBMY3 variant has at least 75 to 80%, more preferably at least 85 to 90%, and even more preferably at least 90% amino acid sequence identity to the amino acid sequence claimed herein, and which retains at least one biological, immunological, or other functional characteristic or activity of HGPRBMY3 polypeptide. Most preferred is a variant having at least 95% amino acid sequence identity to that of SEQ ID NO:2.

In another embodiment, the present invention encompasses polynucleotides, which encode HGPRBMY3 polypeptide. Accordingly, any nucleic acid sequence, which encodes the amino acid sequence of HGPRBMY3 polypeptide, can be used to produce recombinant molecules that express HGPRBMY3 protein. In a particular embodiment, the present invention encompasses the HGPRBMY3 polynucleotide comprising the nucleic acid sequence of SEQ ID NO: 1 and as shown in FIG. 1. More particularly, the present invention provides the HGPRBMY3 clone, deposited at the American Type Culture Collection (ATCC), 10801 University Boulevard, Manassas, Va. 20110-2209 on Nov. 15, 2000 and under ATCC Accession No. PTA-2681 according to the terms of the Budapest Treaty.

As will be appreciated by the skilled practitioner in the art, the degeneracy of the genetic code results in the production of a multitude of nucleotide sequences encoding HGPRBMY3 polypeptide. Some of the sequences bear minimal homology to the nucleotide sequences of any known and naturally occurring gene. Accordingly, the present invention contemplates each and every possible variation of nucleotide sequence that could be made by selecting combinations based on possible codon choices. These combinations are made in accordance with the standard triplet genetic code as applied to the nucleotide sequence of naturally occurring HGPRBMY3, and all such variations are to be considered as being specifically disclosed.

Although nucleotide sequences which encode HGPRBMY3 polypeptide and its variants are preferably capable of hybridizing to the nucleotide sequence of the naturally occurring HGPRBMY3 polypeptide under appropriately selected conditions of stringency, it may be advantageous to produce nucleotide sequences encoding HGPRBMY3 polypeptide, or its derivatives, which possess a substantially different codon usage. Codons may be selected to increase the rate at which expression of the peptide/polypeptide occurs in a particular prokaryotic or eukaryotic host in accordance with the frequency with which particular codons are utilized by the host. Other reasons for substantially altering the nucleotide sequence encoding HGPRBMY3 polypeptide, and its derivatives, without altering the encoded amino acid sequences include the production of RNA transcripts having more desirable properties, such as a greater half-life, than transcripts produced from the naturally occurring sequence.

The present invention also encompasses production of DNA sequences, or portions thereof, which encode the HGPRBMY3 polypeptide, and its derivatives, entirely by synthetic chemistry. After production, the synthetic sequence may be inserted into any of the many available expression vectors and cell systems using reagents that are well known and practiced by those in the art. Moreover, synthetic chemistry may be used to introduce mutations into a sequence encoding HGPRBMY3 polypeptide, or any fragment thereof.

Also encompassed by the present invention are polynucleotide sequences that are capable of hybridizing to the claimed nucleotide sequence of HGPRBMY3, such as that shown in SEQ ID NO: 1, under various conditions of stringency. Hybridization conditions are typically based on the melting temperature (Tₘ) of the nucleic acid binding complex or probe (see, G. M. Wahl and S. L. Berger, 1987; Methods Enzymol., 152:399-407 and A. R. Kimmel, 1987; Methods of Enzymol., 152:507-511), and may be used at a defined stringency. For example, included in the present invention are sequences capable of hybridizing under moderately stringent conditions to the HGPRBMY3 sequence of SEQ ID NO: 1 and other sequences which are degenerate to those which encode HGPRBMY3 polypeptide (e.g., as a non-limiting example: prewashing solution of 2× SSC, 0.5% SDS, 1.0 mM EDTA, pH 8.0, and hybridization conditions of 50° C., 5× SSC, overnight.

The nucleic acid sequence encoding the HGPRBMY3 protein may be extended utilizing a partial nucleotide sequence and employing various methods known in the art to detect upstream sequences such as promoters and regulatory elements. For example, one method, which may be employed, is restriction-site PCR, which utilizes universal primers to retrieve unknown sequence adjacent to a known locus (G. Sarkar, 1993, PCR Methods Applic., 2:318-322). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region or sequence (T. Triglia et al., 1988, Nucleic Acids Res., 16:8186). The primers may be designed using OLIGO 4.06 Primer Analysis software (National Biosciences Inc., Plymouth, Minn.), or another appropriate program, to be 22-30 nucleotides in length, to have a GC content of 50% or more, and to anneal to the target sequence at temperatures about 68°-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome (YAC) DNA (M. Lagerstrom et al., 1991, PCR Methods Applic., 1:111-119). In this method, multiple restriction enzyme digestions and ligations may also be used to place an engineered double-stranded sequence into an unknown portion of the DNA molecule before performing PCR. J. D. Parker et al. (1991; Nucleic Acids Res., 19:3055-3060) provide another method which may be used to retrieve unknown sequences. In addition, PCR, nested primers, and PROMOTERFINDER libraries can be used to walk genomic DNA (Clontech; Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, since they will contain more sequences, which contain the 5′ regions of genes. The use of a randomly primed library may be especially preferable for situations in which an oligo d(T) library does not yield a full-length cDNA. Genomic libraries may be useful for extension of sequence into the 5′ and 3′ non-transcribed regulatory regions.

The embodiments of the present invention can be practiced using methods for DNA sequencing which are well known and generally available in the art. The methods may employ such enzymes as the Klenow fragment of DNA polymerase I, SEQUENASE (US Biochemical Corp.; Cleveland, Ohio), Taq polymerase (PE Biosystems), thermostable T7 polymerase (Amersham Pharmacia Biotech; Piscataway, N.J.), or combinations of recombinant polymerases and proofreading exonucleases such as the ELONGASE Amplification System marketed by Life Technologies (Gaithersburg, Md.). Preferably, the process is automated with machines such as the Hamilton Micro Lab 2200 (Hamilton, Reno, Nev.), Peltier Thermal Cycler (PTC200; MJ Research; Watertown, Mass.) and the ABI Catalyst and 373 and 377 DNA sequencers (PE Biosystems).

Commercially available capillary electrophoresis systems may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled device camera. Output/light intensity may be converted to electrical signal using appropriate software (e.g., GENOTYPER and SEQUENCE NAVIGATOR, PE Biosystems) and the entire process—from loading of samples to computer analysis and electronic data display—may be computer controlled. Capillary electrophoresis is especially preferable for the sequencing of small pieces of DNA, which may be present in limited amounts in a particular sample.

In another embodiment of the present invention, polynucleotide sequences or fragments thereof which encode HGPRBMY3 polypeptide, or peptides thereof, may be used in recombinant DNA molecules to direct the expression of HGPRBMY3 polypeptide product, or fragments or functional equivalents thereof, in appropriate host cells. Because of the inherent degeneracy of the genetic code, other DNA sequences, which encode substantially the same or a functionally equivalent amino acid sequence, may be produced and these sequences may be used to clone and express HGPRBMY3 protein.

As will be appreciated by those having skill in the art, it may be advantageous to produce HGPRBMY3 polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The nucleotide sequence of the present invention can be engineered using methods generally known in the art in order to alter HGPRBMY3 polypeptide-encoding sequences for a variety of reasons, including, but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and the like.

In preferred embodiments, the present invention encompasses a polynucleotide lacking the initiating start codon, in addition to, the resulting encoded polypeptide of HGPRBMY3. Specifically, the present invention encompasses the polynucleotide corresponding to nucleotides 4 thru 1116 of SEQ ID NO: 1, and the polypeptide corresponding to amino acids 2 thru 372 of SEQ ID NO:2. Also encompassed are recombinant vectors comprising said encoding sequence, and host cells comprising said vector.

In another embodiment of the present invention, natural, modified, or recombinant nucleic acid sequences encoding HGPRBMY3 polypeptide may be ligated to a heterologous sequence to encode a fusion protein. For example, for screening peptide libraries for inhibitors of HGPRBMY3 activity, it may be useful to encode a chimeric HGPRBMY3 protein that can be recognized by a commercially available antibody. A fusion protein may also be engineered to contain a cleavage site located between the HGPRBMY3 protein-encoding sequence and the heterologous protein sequence, so that HGPRBMY3 protein may be cleaved and purified away from the heterologous moiety.

In another embodiment, sequences encoding HGPRBMY3 polypeptide may be synthesized in whole, or in part, using chemical methods well known in the art (see, for example, M. H. Caruthers et al., 1980, Nucl. Acids Res. Symp. Ser., 215-223 and T. Horn et al., 1980, Nucl. Acids Res. Symp. Ser., 225-232). Alternatively, the protein itself may be produced using chemical methods to synthesize the amino acid sequence of HGPRBMY3 polypeptide, or a fragment or portion thereof. For example, peptide synthesis can be performed using various solid-phase techniques (J. Y. Roberge et al., 1995, Science, 269:202-204) and automated synthesis may be achieved, for example, using the ABI 431 A Peptide Synthesizer (PE Biosystems).

The newly synthesized peptide can be substantially purified by preparative high performance liquid chromatography (e.g., T. Creighton, 1983, Proteins, Structures and Molecular Principles, W.H. Freeman and Co., New York, N.Y.), by reversed-phase high performance liquid chromatography, or other purification methods as are known in the art. The composition of the synthetic peptides may be confirmed by amino acid analysis or sequencing (e.g., the Edman degradation procedure; Creighton, supra). In addition, the amino acid sequence of HGPRBMY3 polypeptide or any portion thereof, may be altered during direct synthesis and/or combined using chemical methods with sequences from other proteins, or any part thereof, to produce a variant polypeptide.

To express a biologically active HGPRBMY3 polypeptide or peptide, the nucleotide sequences encoding HGPRBMY3 polypeptide, or functional equivalents, may be inserted into an appropriate expression vector, i.e., a vector, which contains the necessary elements for the transcription and translation of the inserted coding sequence.

Methods, which are well known to those skilled in the art, may be used to construct expression vectors containing sequences encoding HGPRBMY3 polypeptide and appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector/host systems may be utilized to contain and express sequences encoding HGPRBMY3 polypeptide. Such expression vector/host systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); or animal cell systems. The host cell employed is not limiting to the present invention.

“Control elements” or “regulatory sequences” are those non-translated regions of the vector, e.g., enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene; La Jolla, Calif.) or PSPORT1 plasmid (Life Technologies), and the like, may be used. The baculovirus polyhedrin promoter may be used in insect cells. Promoters or enhancers derived from the genomes of plant cells (e.g., heat shock, RUBISCO; and storage protein genes), or from plant viruses (e.g., viral promoters or leader sequences), may be cloned into the vector. In mammalian cell systems, promoters from mammalian genes or from mammalian viruses are preferred. If it is necessary to generate a cell line that contains multiple copies of the sequence encoding HGPRBMY3, vectors based on SV40 or EBV may be used with an appropriate selectable marker.

In bacterial systems, a number of expression vectors may be selected, depending upon the use intended for the expressed HGPRBMY3 product. For example, when large quantities of expressed protein are needed for the induction of antibodies, vectors, which direct high level expression of fusion proteins that are readily purified, may be used. Such vectors include, but are not limited to, the multifunctional E. coli cloning and expression vectors such as BLUESCRIPT (Stratagene), in which the sequence encoding HGPRBMY3 polypeptide may be ligated into the vector in-frame with sequences for the amino-terminal Met and the subsequent 7 residues of β-galactosidase, so that a hybrid protein is produced; pIN vectors (see, G. Van Heeke and S. M. Schuster, 1989.J. Biol. Chem., 264:5503-5509); and the like. pGEX vectors (Promega; Madison, Wis.) may also be used to express foreign polypeptides, as fusion proteins with glutathione S-transferase (GST). In general, such fusion proteins are soluble and can be easily purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. (For reviews, see F. M. Ausubel et al., supra, and Grant et al., 1987, Methods Enzymol., 153:516-544).

Should plant expression vectors be desired and used, the expression of sequences encoding HGPRBMY3 polypeptide may be driven by any of a number of promoters. For example, viral promoters such as the 35S and 19S promoters of CaMV may be used alone or in combination with the omega leader sequence from TMV (N. Takamatsu, 1987, EMBO J, 6:307-311). Alternatively, plant promoters such as the small subunit of RUBISCO, or heat shock promoters, may be used (G. Coruzzi et al., 1984, EMBO J, 3:1671-1680; R. Broglie et al., 1984, Science, 224:838-843; and J. Winter et al., 1991, Results Probl. Cell Differ. 17:85-105). These constructs can be introduced into plant cells by direct DNA transformation or pathogen-mediated transfection. Such techniques are described in a number of generally available reviews (see, for example, S. Hobbs or L. E. Murry, In: McGraw Hill Yearbook of Science and Technology (1992) McGraw Hill, New York, N.Y.; pp. 191-196).

An insect system may also be used to express HGPRBMY3 polypeptide. For example, in one such system, Autographa californica nuclear polyhedrosis virus (AcNPV) is used as a vector to express foreign genes in Spodopterafrugiperda cells or in Trichoplusia larvae. The sequences encoding HGPRBMY3 polypeptide may be cloned into a non-essential region of the virus such as the polyhedrin gene and placed under control of the polyhedrin promoter. Successful insertion of HGPRBMY3 polypeptide will render the polyhedrin gene inactive and produce recombinant virus lacking coat protein. The recombinant viruses may then be used to infect, for example, S. frugiperda cells or Trichoplusia larvae in which the HGPRBMY3 polypeptide product may be expressed (E. K. Engelhard et al., 1994, Proc. Nat. Acad. Sci., 91:3224-3227).

In mammalian host cells, a number of viral-based expression systems may be utilized. In cases where an adenovirus is used as an expression vector, sequences encoding HGPRBMY3 polypeptide may be ligated into an adenovirus transcription/translation complex containing the late promoter and tripartite leader sequence. Insertion in a non-essential E1 or E3 region of the viral genome may be used to obtain a viable virus which is capable of expressing HGPRBMY3 polypeptide in infected host cells (J. Logan and T. Shenk, 1984, Proc. Natl. Acad. Sci., 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding HGPRBMY3 polypeptide. Such signals include the ATG initiation codon and adjacent sequences. In cases where sequences encoding HGPRBMY3 polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals, including the ATG initiation codon, should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system that is used, such as those described in the literature (D. Scharf et al., 1994, Results Probl. Cell Differ., 20:125-162).

Moreover, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells having specific cellular machinery and characteristic mechanisms for such post-translational activities (e.g., CHO, HeLa, MDCK, HEK293, and W138) are available from the American Type Culture Collection (ATCC), American Type Culture Collection (ATCC), 10801 University Boulevard, Manassas, Va. 20110-2209, and may be chosen to ensure the correct modification and processing of the foreign protein.

For long-term, high-yield production of recombinant proteins, stable expression is preferred. For example, cell lines which stably express HGPRBMY3 protein may be transformed using expression vectors, which may contain viral origins of replication and/or endogenous expression elements, and a selectable marker gene on the same, or on a separate, vector. Following the introduction of the vector, cells may be allowed to grow for 1-2 days in an enriched cell culture medium before they are switched to selective medium. The purpose of the selectable marker is to confer resistance to selection, and its presence allows the growth and recovery of cells, which successfully express the introduced sequences. Resistant clones of stably transformed cells may be proliferated using tissue culture techniques appropriate to the cell type.

Any number of selection systems may be used to recover transformed cell lines. These include, but are not limited to, the Herpes Simplex Virus thymidine kinase (HSV TK), (M. Wigler et al., 1977, Cell, 11:223-32) and adenine phosphoribosyltransferase (I. Lowy et al., 1980, Cell, 22:817-23) genes which can be employed in tk⁻ or aprt⁻ cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G-418 (F. Colbere-Garapin et al., 1981, J Mol. Biol., 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (S. C. Hartman and R. C. Mulligan, 1988, Proc. Natl. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C. A. Rhodes et al., 1995, Methods Mol. Biol., 55:121-131).

Although the presence or absence of marker gene expression suggests that the gene of interest is also present, the presence and expression of the desired gene of interest may need to be confirmed. For example, if the nucleic acid sequence encoding HGPRBMY3 polypeptide is inserted within a marker gene sequence, recombinant cells containing sequences encoding HGPRBMY3 polypeptide can be identified by the absence of marker gene function. Alternatively, a marker gene can be placed in tandem with a sequence encoding HGPRBMY3 polypeptide under the control of a single promoter. Expression of the marker gene in response to induction or selection usually indicates co-expression of the tandem gene.

Alternatively, host cells, which contain the nucleic acid, sequence encoding HGPRBMY3 polypeptide and which express HGPRBMY3 polypeptide product may be identified by a variety of procedures known to those having skill in the art. These procedures include, but are not limited to, DNA-DNA or DNA-RNA hybridizations and protein bioassay or immunoassay techniques, including membrane, solution, or chip based technologies, for the detection and/or quantification of nucleic acid or protein.

The presence of polynucleotide sequences encoding HGPRBMY3 polypeptide can be detected by DNA-DNA or DNA-RNA hybridization, or by amplification using probes or portions or fragments of polynucleotides encoding HGPRBMY3 polypeptide. Nucleic acid amplification based assays involve the use of oligonucleotides or oligomers, based on the sequences encoding HGPRBMY3 polypeptide, to detect transformants containing DNA or RNA encoding HGPRBMY3 polypeptide.

A wide variety of labels and conjugation techniques are known and employed by those skilled in the art and may be used in various nucleic acid and amino acid assays. Means for producing labeled hybridization or PCR probes for detecting sequences related to polynucleotides encoding HGPRBMY3 polypeptide include oligo-labeling, nick translation, end-labeling, or PCR amplification using a labeled nucleotide. Alternatively, the sequences encoding HGPRBMY3 polypeptide, or any portions or fragments thereof, may be cloned into a vector for the production of an mRNA probe. Such vectors are known in the art, are commercially available, and may be used to synthesize RNA probes in vitro by addition of an appropriate RNA polymerase, such as T7, T3, or SP(6) and labeled nucleotides. These procedures may be conducted using a variety of commercially available kits (e.g., Amersham Pharmacia Biotech, Promega and U.S. Biochemical Corp.). Suitable reporter molecules or labels which may be used include radionuclides, enzymes, fluorescent, chemiluminescent, or chromogenic agents, as well as substrates, cofactors, inhibitors, magnetic particles, and the like.

Host cells transformed with nucleotide sequences encoding HGPRBMY3 protein, or fragments thereof, may be cultured under conditions suitable for the expression and recovery of the protein from cell culture. The protein produced by a recombinant cell may be secreted or contained intracellularly depending on the sequence and/or the vector used. As will be understood by those having skill in the art, expression vectors containing polynucleotides which encode HGPRBMY3 protein may be designed to contain signal sequences which direct secretion of the HGPRBMY3 protein through a prokaryotic or eukaryotic cell membrane. Other constructions may be used to join nucleic acid sequences encoding HGPRBMY3 protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAGS extension/affinity purification system (Immunex Corp.; Seattle, Wash.). The inclusion of cleavable linker sequences such as those specific for Factor XA or enterokinase (Invitrogen; San Diego, Calif.) between the purification domain and HGPRBMY3 protein may be used to facilitate purification. One such expression vector provides for expression of a fusion protein containing HGPRBMY3 and a nucleic acid encoding 6 histidine residues preceding a thioredoxin or an enterokinase cleavage site. The histidine residues facilitate purification on IMAC (immobilized metal ion affinity chromatography) as described by J. Porath et al., 1992, Prot. Exp. Purif., 3:263-281, while the enterokinase cleavage site provides a means for purifying from the fusion protein. For a discussion of suitable vectors for fusion protein production, see D. J. Kroll et al., 1993; DNA Cell Biol., 12:441-453.

In addition to recombinant production, fragments of HGPRBMY3 polypeptide may be produced by direct peptide synthesis using solid-phase techniques (J. Merrifield, 1963, J. Am. Chem. Soc., 85:2149-2154). Protein synthesis maybe performed using manual techniques or by automation. Automated synthesis may be achieved, for example, using ABI 431A Peptide Synthesizer (PE Biosystems). Various fragments of HGPRBMY3 polypeptide can be chemically synthesized separately and then combined using chemical methods to produce the full length molecule.

Human artificial chromosomes (HACs) may be used to deliver larger fragments of DNA than can be contained and expressed in a plasmid vector. HACs are linear microchromosomes which may contain DNA sequences of 10K to 10M in size, and contain all of the elements that are required for stable mitotic chromosome segregation and maintenance (see, J. J. Harrington et al., 1997, Nature Genet., 15:345-355). HACs of 6 to 10M are constructed and delivered via conventional delivery methods (e.g., liposomes, polycationic amino polymers, or vesicles) for therapeutic purposes.

Diagnostic Assays

A variety of protocols for detecting and measuring the expression of HGPRBMY3 polypeptide using either polyclonal or monoclonal antibodies specific for the protein are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on the HGPRBMY3 polypeptide is preferred, but a competitive binding assay may also be employed. These and other assays are described in the art as represented by the publication of R. Hampton et al., 1990; Serological Methods, a Laboratory Manual, APS Press, St Paul, Minn. and D. E. Maddox et al., 1983; J Exp. Med., 158:1211-1216).

This invention also relates to the use of HGPRBMY3 polynucleotides as diagnostic reagents. Detection of a mutated form of the HGPRBMY3 gene associated with a dysfunction will provide a diagnostic tool that can add to or define a diagnosis of a disease or susceptibility to a disease which results from under-expression, over-expression, or altered expression of HGPRBMY3. Individuals carrying mutations in the HGPRBMY3 gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Hybridizing amplified DNA to labeled HGPRBMY3 polynucleotide sequences can identify point mutations. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al., Science (1985) 230:1242. Sequence changes at specific locations may also be revealed by nuclease protection assays, such as RNase and SI protection or the chemical cleavage method (see Cotton et al., Proc. Natl. Acad. Sci., USA (1985) 85:43297-4401). In another embodiment, an array of oligonucleotides probes comprising HGPRBMY3 nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, 274:610-613, 1996).

The diagnostic assays offer a process for diagnosing or determining a susceptibility to infections such as bacterial, fungal, protozoan and viral infections, particularly infections caused by HIV-1 or HIV-2 through detection of a mutation in the HGPRBMY3 gene by the methods described. The invention also provides diagnostic assays for determining or monitoring susceptibility to the following conditions, diseases, or disorders: cancers; anorexia; bulimia asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, schizophrenia, manic depression, delirium, dementia, severe mental retardation and dyskinesias, such as Huntington's disease or Gilles dela Tourett's syndrome.

In addition, infections such as bacterial, protozoan and viral infections, particularly infections caused by HIV-1 or HIV-2; as well as, conditions or disorders such as pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, schizophrenia, manic depression, delirium, dementia, severe mental retardation and dyskinesias, such as Huntington's disease or Gilles dela Tourett's syndrome, can be diagnosed by methods comprising determining from a sample derived from a subject having an abnormally decreased or increased level of HGPRBMY3 polypeptide or HGPRBMY3 mRNA. Decreased or increased expression can be measured at the RNA level using any of the methods well known in the art for the quantification of polynucleotides, such as, for example, PCR, RT-PCR, RNase protection, Northern blotting and other hybridization methods. Assay techniques that can be used to determine levels of a protein, such as an HGPRBMY3, in a sample derived from a host are well-known to those of skill in the art. Such assay methods include radioimmunoassays, competitive-binding assays, Western Blot analysis and ELISA assays.

In another of its aspects, the present invention relates to a diagnostic kit for a disease or susceptibility to a disease, particularly infections such as bacterial, fungal, protozoan and viral infections, particularly infections caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy, and psychotic and neurological disorders, including anxiety, schizophrenia, manic depression, delirium, dementia, severe medal retardation and dyskinesias, such as Huntington's disease or Gilles dela Tourett's syndrome, which comprises:

(a) a HGPRBMY3 polynucleotide, preferably the nucleotide sequence of SEQ ID NO: 1, or a fragment thereof, or

(b) a nucleotide sequence complementary to that of (a); or

(c) a HGPRBMY3 polypeptide, preferably the polypeptide of SEQ ID NO: 2, or a fragment thereof; or

(d) an antibody to a HGPRBMY3 polypeptide, preferably to the polypeptide of SEQ ID NO: 2, or combinations thereof.

It will be appreciated that in any such kit, (a), (b), (c) or (d) may comprise a substantial component.

The GPCR polynucleotides, which may be used in the diagnostic assays according to the present invention, include oligonucleotide sequences, complementary RNA and DNA molecules, and PNAs. The polynucleotides may be used to detect and quantify HGPRBMY3-encoding nucleic acid expression in biopsied tissues in which expression (or under- or overexpression) of the HGPRBMY3 polynucleotide may be correlated with disease. The diagnostic assays may be used to distinguish between the absence, presence, and excess expression of HGPRBMY3, and to monitor regulation of HGPRBMY3 polynucleotide levels during therapeutic treatment or intervention.

In a related aspect, hybridization with PCR probes which are capable of detecting polynucleotide sequences, including genomic sequences, encoding HGPRBMY3 polypeptide, or closely related molecules, may be used to identify nucleic acid sequences which encode HGPRBMY3 polypeptide. The specificity of the probe, whether it is made from a highly specific region, e.g., about 8 to 10 contiguous nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding HGPRBMY3 polypeptide, alleles thereof, or related sequences.

Probes may also be used for the detection of related sequences, and should preferably contain at least 50% of the nucleotides encoding the HGPRBMY3 polypeptide. The hybridization probes of this invention may be DNA or RNA and may be derived from the nucleotide sequence of SEQ ID NO: 1, or from genomic sequence including promoter, enhancer elements, and introns of the naturally occurring HGPRBMY3 protein.

Methods for producing specific hybridization probes for DNA encoding the HGPRBMY3 polypeptide include the cloning of a nucleic acid sequence that encodes the HGPRBMY3 polypeptide, or HGPRBMY3 derivatives, into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of detector/reporter groups, e.g., radionuclides such as ³²P or ³⁵S, or enzymatic labels, such as alkaline phosphatase coupled to the probe via avidin/biotin coupling systems, and the like.

The polynucleotide sequence encoding the HGPRBMY3 polypeptide, or fragments thereof, may be used for the diagnosis of disorders associated with expression of HGPRBMY3. Examples of such disorders or conditions are described for “Therapeutic Assays”. The polynucleotide sequence encoding the HGPRBMY3 polypeptide may be used in Southern or Northern analysis, dot blot, or other membrane-based technologies; in PCR technologies; or in dip stick, pin, ELISA or chip assays utilizing fluids or tissues from patient biopsies to detect the status of, e.g., levels or overexpression of HGPRBMY3, or to detect altered HGPRBMY3 expression. Such qualitative or quantitative methods are well known in the art.

In a particular aspect, the nucleotide sequence encoding the HGPRBMY3 polypeptide may be useful in assays that detect activation or induction of various neoplasms or cancers, particularly those mentioned supra. The nucleotide sequence encoding the HGPRBMY3 polypeptide may be labeled by standard methods, and added to a fluid or tissue sample from a patient, under conditions suitable for the formation of hybridization complexes. After a suitable incubation period, the sample is washed and the signal is quantified and compared with a standard value. If the amount of signal in the biopsied or extracted sample is significantly altered from that of a comparable control sample, the nucleotide sequence has hybridized with nucleotide sequence present in the sample, and the presence of altered levels of nucleotide sequence encoding the HGPRBMY3 polypeptide in the sample indicates the presence of the associated disease. Such assays may also be used to evaluate the efficacy of a particular therapeutic treatment regimen in animal studies, in clinical trials, or in monitoring the treatment of an individual patient.

To provide a basis for the diagnosis of disease associated with expression of HGPRBMY3, a normal or standard profile for expression is established. This may be accomplished by combining body fluids or cell extracts taken from normal subjects, either animal or human, with a sequence, or a fragment thereof, which encodes the HGPRBMY3 polypeptide, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject (patient) values is used to establish the presence of disease.

Once disease is established and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to cancer, the presence of an abnormal amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the nucleic acid sequence encoding the HGPRBMY3 polypeptide may involve the use of PCR. Such oligomers may be chemically synthesized, generated enzymatically, or produced from a recombinant source. Oligomers will preferably comprise two nucleotide sequences, one with sense orientation (5′→3′) and another with antisense (3′→5′), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods suitable for quantifying the expression of HGPRBMY3 include radiolabeling or biotinylating nucleotides, co-amplification of a control nucleic acid, and standard curves onto which the experimental results are interpolated (P. C. Melby et al., 1993, J. Immunol. Methods, 159:235-244; and C. Duplaa et al., 1993, Anal. Biochem., 229-236). The speed of quantifying multiple samples may be accelerated by running the assay in an ELISA format where the oligomer of interest is presented in various dilutions and a spectrophotometric or calorimetric response gives rapid quantification.

Therapeutic Assays

The HGPRBMY3 polypeptide (SEQ ID NO:2) shares homology with a chicken Purinergic receptor (P2Y5; SEQ ID NO:12). The HGPRBMY3 protein may play a role in immune, testicular, colonic, breast, and ovarian disorders, and/or in cell cycle regulation, and/or in cell signaling. The HGPRBMY3 protein may further be involved in neoplastic, neurological, and immunological disorders.

In one embodiment of the present invention, the HGPRBMY3 protein may play a role in neoplastic disorders. An antagonist of HGPRBMY3 polypeptide may be administered to an individual to prevent or treat a neoplastic disorder. Such disorders may include, but are not limited to, adenocarcinoma, leukemia, lymphoma, melanoma, myeloma, sarcoma, and teratocarcinoma, and particularly, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In a related aspect, an antibody which specifically binds to HGPRBMY3 may be used directly as an antagonist or indirectly as a targeting or delivery mechanism for bringing a pharmaceutical agent to cells or tissue which express the HGPRBMY3 polypeptide.

In another embodiment of the present invention, an antagonist or inhibitory agent of the HGPRBMY3 polypeptide may be administered to an individual to prevent or treat a neurological disorder. Such disorders may include, but are not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder.

In a preferred embodiment of the present invention, an antagonist or inhibitory agent of the HGPRBMY3 polypeptide may be administered to an individual to prevent or treat an immunological disorder, particularly since HGPRBMY3 is highly expressed in immune-, testes-, colon-, and breast-related tissues. Such conditions or disorders may include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, leukemia, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections, and trauma.

In preferred embodiments, the HGPRBMY3 polynucleotides and polypeptides, including agonists, antagonists, and fragments thereof, are useful for modulating intracellular Ca²⁺ levels, modulating Ca²⁺ sensitive signaling pathways, and modulating NFAT element associated signaling pathways.

In yet another embodiment of the present invention, an expression vector containing the complement of the polynucleotide encoding HGPRBMY3 polypeptide may be administered to an individual to treat or prevent a neoplastic disorder, including, but not limited to, the types of cancers and tumors described above. For example, but not limited to testes, colon, breast, and/or ovarian cancer.

In a further embodiment of the present invention, an expression vector containing the complement of the polynucleotide encoding HGPRBMY3 polypeptide may be administered to an individual to treat or prevent a neurological disorder, including, but not limited to, the types of disorders described above.

In yet another embodiment of the present invention, an expression vector containing the complement of the polynucleotide encoding HGPRBMY3 polypeptide may be administered to an individual to treat or prevent an immune disorder, including, but not limited to, the types of immune disorders described above, testicular, colonic, breast, and ovarian diseases, disorders, and conditions.

In a further embodiment of the present invention, an expression vector harboring the complement of the polynucleotide encoding HGPRBMY3 polypeptide may be administered to an individual to treat or prevent a developmental disorder, including, but not limited to, the types of disorders described above.

In another embodiment, the proteins, antagonists, antibodies, agonists, complementary sequences, or vectors of the present invention can be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Antagonists or inhibitors of the HGPRBMY3 polypeptide of the present invention may be produced using methods which are generally known in the art. For example, HGPRBMY3 transfected CHO-NFAT/CRE cell lines of the present invention are useful for the identification of agonists and antagonists of the HGPRBMY3 polypeptide. Representative uses of these cell lines would be their inclusion in a method of identifying HGPRBMY3 agonists and antagonists. Preferably, the cell lines are useful in a method for identifying a compound that modulates the biological activity of the HGPRBMY3 polypeptide, comprising the steps of (a) combining a candidate modulator compound with a host cell expressing the HGPRBMY3 polypeptide having the sequence as set forth in SEQ ID NO:2; and (b) measuring an effect of the candidate modulator compound on the activity of the expressed HGPRBMY3 polypeptide. Representative vectors expressing the HGPRBMY3 polypeptide are referenced herein (e.g., pcDNA3.1 hygro™) or otherwise known in the art.

The cell lines are also useful in a method of screening for a compounds that is capable of modulating the biological activity of HGPRBMY3 polypeptide, comprising the steps of: (a) determining the biological activity of the HGPRBMY3 polypeptide in the absence of a modulator compound; (b) contacting a host cell expression the HGPRBMY3 polypeptide with the modulator compound; and (c) determining the biological activity of the HGPRBMY3 polypeptide in the presence of the modulator compound; wherein a difference between the activity of the HGPRBMY3 polypeptide in the presence of the modulator compound and in the absence of the modulator compound indicates a modulating effect of the compound. Additional uses for these cell lines are described herein or otherwise known in the art. In particular, purified HGPRBMY3 protein, or fragments thereof, can be used to produce antibodies, or to screen libraries of pharmaceutical agents, to identify those which specifically bind HGPRBMY3.

Antibodies specific for HGPRBMY3 polypeptide, or immunogenic peptide fragments thereof, can be generated using methods that have long been known and conventionally practiced in the art. Such antibodies may include, but are not limited to, polyclonal, monoclonal, chimeric, single chain, Fab fragments, and fragments produced by an Fab expression library. Neutralizing antibodies, (i.e., those which inhibit dimer formation) are especially preferred for therapeutic use.

The present invention also encompasses the polypeptide sequences that intervene between each of the predicted HGPRBMY3 transmembrane domains. Since these regions are solvent accessible either extracellularly or intracellularly, they are particularly useful for designing antibodies specific to each region. Such antibodies may be useful as antagonists or agonists of the HGPRBMY3 full-length polypeptide and may modulate its activity.

The following serve as non-limiting examples of peptides or fragments that may be used to generate antibodies:

(SEQ ID NO:17)

MLANSSSTNSSVLPCPDYRPTHR

(SEQ ID NO:18)

RALRVHSVVS

(SEQ ID NO:19)

HHWPFPDLLCQTTG

(SEQ ID NO:20)

DRYAAIVHPLRLRHLRRPR

(SEQ ID NO:21)

AARVHRPSRCRYRDLEVRLCFESFSDELWKGRLLP

(SEQ ID NO:22)

RVFWTLARPDATQSQRRRKTVRL

(SEQ ID NO:23)

VYGLLRSKLVAASVPARDRVR

(SEQ ID NO:24)

AEGFRNTLRGLGTPHRARTSATNGTRAALAQSERSAVTTDATRPDAASQG
LLRPSDSHSLSSFTQCPQDSAL

In preferred embodiments, the following N-terminal HGPRBMY3 deletion polypeptides are encompassed by the present invention: M1-R23, L2-R23, A3-R23, N4-R23, S5-R23, S6-R23, S7-R23, T8-R23, N9-R23, S10-R23, S11-R23, V12-R23, L13-R23, P14-R23, C15-R23, P16-R23, and/or D17-R23 of SEQ ID NO:17. Polynucleotide sequences encoding these polypeptides are also provided. The present invention also encompasses the use of these N-terminal HGPRBMY3 deletion polypeptides as immunogenic and/or antigenic epitopes as described elsewhere herein.

In preferred embodiments, the following C-terminal HGPRBMY3 deletion polypeptides are encompassed by the present invention: M1-R23, M1-H22, M1-T21, M1-P20, M1-R19, M1-Y18, M1-D17, M1-P16, M1-C15, M1-P14, M1-L13, M1-V12, M1-S11, M1-S10, M1-N9, M1-T8, and/or M1-S7 of SEQ ID NO:17. Polynucleotide sequences encoding these polypeptides are also provided. The present invention also encompasses the use of these C-terminal HGPRBMY3 deletion polypeptides as immunogenic and/or antigenic epitopes as described elsewhere herein.

In preferred embodiments, the following N-terminal HGPRBMY3 TM1-2 intertransmembrane domain deletion polypeptides are encompassed by the present invention: R1-S10, A2-S10, L3-S10, and/or R4-S10 of SEQ ID NO:18. Polynucleotide sequences encoding these polypeptides are also provided. The present invention also encompasses the use of these N-terminal HGPRBMY3 TM1-2 intertransmembrane domain deletion polypeptides as immunogenic and/or antigenic epitopes as described elsewhere herein.

In preferred embodiments, the following C-terminal HGPRBMY3 TM1-2 intertransmembrane domain deletion polypeptides are encompassed by the present invention: R1-S10, R1-V9, R1-V8, and/or R1-S7 of SEQ ID NO: 18. Polynucleotide sequences encoding these polypeptides are also provided. The present invention also encompasses the use of these C-terminal HGPRBMY3 TM1-2 intertransmembrane domain deletion polypeptides as immunogenic and/or antigenic epitopes as described elsewhere herein.

In preferred embodiments, the following N-terminal HGPRBMY3 TM2-3 intertransmembrane domain deletion polypeptides are encompassed by the present invention: H1-G14, H2-G14, W3-G14, P4-G14, F5-G14, P6-G14, D7-G14, and/or L8-G14 of SEQ ID NO:19. Polynucleotide sequences encoding these polypeptides are also provided. The present invention also encompasses the use of these N-terminal HGPRBMY3 TM2-3 intertransmembrane domain deletion polypeptides as immunogenic and/or antigenic epitopes as described elsewhere herein.

In preferred embodiments, the following C-terminal HGPRBMY3 TM2-3 intertransmembrane domain deletion polypeptides are encompassed by the present invention: H1-G14, H1-T13, H1-T12, H1-Q11, H1-C10, H1-L9, H1-L8, and/or H1-D7 of SEQ ID NO:19. Polynucleotide sequences encoding these polypeptides are also provided. The present invention also encompasses the use of these C-terminal HGPRBMY3 TM2-3 inteirtransmembrane domain deletion polypeptides as immunogenic and/or antigenic epitopes as described elsewhere herein.

In preferred embodiments, the following N-terminal HGPRBMY3 TM3-4 intertransmembrane domain deletion polypeptides are encompassed by the present invention: D1-R19, R2-R19, Y3-R19, A4-R19, A5-R19, 16-R19, V7-R19, H8-R19, P9-R19, L10-R19, R11-R19, L12-R19, and/or R13-R19 of SEQ ID NO:20. Polynucleotide sequences encoding these polypeptides are also provided. The present invention also encompasses the use of these N-terminal HGPRBMY3 TM3-4 intertransmembrane domain deletion polypeptides as immunogenic and/or antigenic epitopes as described elsewhere herein.

In preferred embodiments, the following C-terminal HGPRBMY3 TM3-4 intertransmembrane domain deletion polypeptides are encompassed by the present invention: D1-R19, D1-P18, D1-R17, D1-R16, D1-L15, D1-H14, D1-R13, D1-L12, D1-R11, D1-L10, D1-P9, D1-H8, and/or D1-V7 of SEQ ID NO:20. Polynucleotide sequences encoding these polypeptides are also provided. The present invention also encompasses the use of these C-terminal HGPRBMY3 TM3-4 intertransmembrane domain deletion polypeptides as immunogenic and/or antigenic epitopes as described elsewhere herein.

In preferred embodiments, the following N-terminal HGPRBMY3 TM4-5 intertransmembrane domain deletion polypeptides are encompassed by the present invention: A1-P35, A2-P35, R3-P35, V4-P35, H5-P35, R6-P35, P7-P35, S8-P35, R9-P35, C10-P35, R11-P35, Y12-P35, R13-P35, D14-P35, L15-P35, E16-P35, V17-P35, R18-P35, L19-P35, C20-P35, F21-P35, E22-P35, S23-P35, F24-P35, S25-P35, D26-P35, E27-P35, L28-P35, and/or W29-P35 of SEQ ID NO:21. Polynucleotide sequences encoding these polypeptides are also provided. The present invention also encompasses the use of these N-terminal HGPRBMY3 TM4-5 intertransmembrane domain deletion polypeptides as immunogenic and/or antigenic epitopes as described elsewhere herein.

In preferred embodiments, the following C-terminal HGPRBMY3 TM4-5 intertransmembrane domain deletion polypeptides are encompassed by the present invention: A1-P35, A1-L34, A1-L33, A1-R32, A1-G31, A1-K30, A1-W29, A1-L28, A1-E27, A1-D26, A1-S25, A1-F24, A1-S23, A1-E22, A1-F21, A1-C20, Al-L19, A1-R18, A1-V17, A1-E16, A1-L15, A1-D14, A1-R13, A1-Y12, A1-R11, A1-C10, A1-R9, A1-S8, and/or A1-P7 of SEQ ID NO:21. Polynucleotide sequences encoding these polypeptides are also provided. The present invention also encompasses the use of these C-terminal HGPRBMY3 TM4-5 intertransmembrane domain deletion polypeptides as immunogenic and/or antigenic epitopes as described elsewhere herein.

In preferred embodiments, the following N-terminal HGPRBMY3 TM5-6 intertransmembrane domain deletion polypeptides are encompassed by the present invention: R1-L23, V2-L23, F3-L23, W4-L23, T5-L23, L6-L23, A7-L23, R8-L23, P9-L23, D10-L23, A11-L23, T12-L23, Q13-L23, S14-L23, Q15-L23, R16-L23, and/or R17-L23 of SEQ ID NO:22. Polynucleotide sequences encoding these polypeptides are also provided. The present invention also encompasses the use of these N-terminal HGPRBMY3 TM5-6 intertransmembrane domain deletion polypeptides as immunogenic and/or antigenic epitopes as described elsewhere herein.

In preferred embodiments, the following C-terminal HGPRBMY3 TM5-6 intertransmembrane domain deletion polypeptides are encompassed by the present invention: R1-L23, R1-R22, R1-V21, R1-T20, R1-K19, R1-R18, R1-R17, R1-R16, R1-Q15, R1-S14, R1-Q13, R1-T12, R1-A11, R1-D10, R1-P9, R1-R8, and/or R1-A7 of SEQ ID NO:22. Polynucleotide sequences encoding these polypeptides are also provided. The present invention also encompasses the use of these C-terminal HGPRBMY3 TM5-6 intertransmembrane domain deletion polypeptides as immunogenic and/or antigenic epitopes as described elsewhere herein.

In preferred embodiments, the following N-terminal HGPRBMY3 TM6-7 intertransmembrane domain deletion polypeptides are encompassed by the present invention: V1-R21, Y2-R21, G3-R21, L4-R21, L5-R21, R6-R21, S7-R21, K8-R21, L9-R21, V10-R21, A11-R21, A12-R21, S13-R21, V14-R21, and/or P15-R21 of SEQ ID NO:23. Polynucleotide sequences encoding these polypeptides are also provided. The present invention also encompasses the use of these N-terminal HGPRBMY3 TM6-7 intertransmembrane domain deletion polypeptides as immunogenic and/or antigenic epitopes as described elsewhere herein.

In preferred embodiments, the following C-terminal HGPRBMY3 TM6-7 intertransmembrane domain deletion polypeptides are encompassed by the present invention: V1-R21, V1-V20, VI-R19, V1-D18, V1-R17, V1-A16, V1-P15, V1-V14, V1-S13, V1-A12, V1-A11, V1-V10, V1-L9, V1-K8, and/or V1-S7 of SEQ ID NO:23. Polynucleotide sequences encoding these polypeptides are also provided. The present invention also encompasses the use of these C-terminal HGPRBMY3 TM6-7 intertransmembrane domain deletion polypeptides as immunogenic and/or antigenic epitopes as described elsewhere herein.

In preferred embodiments, the following N-terminal HGPRBMY3 C-terminal fragment deletion polypeptides are encompassed by the present invention: A1-L72, E2-L72, G3-L72, F4-L72, R5-L72, N6-L72, T7-L72, L8-L72, R9-L72, G10-L72, L11-L72, G12-L72, T13-L72, P14-L72, H15-L72, R16-L72, A17-L72, R18-L72, T19-L72, S20-L72, A21-L72, T22-L72, N23-L72, G24-L72, T25-L72, R26-L72, A27-L72, A28-L72, L29-L72, A30-L72, Q31-L72, S32-L72, E33-L72, R34-L72, S35-L72, A36-L72, V37-L72, T38-L72, T39-L72, D40-L72, A41-L72, T42-L72, R43-L72, P44-L72, D45-L72, A46-L72, A47-L72, S48-L72, Q49-L72, G50-L72, L51-L72, L52-L72, R53-L72, P54-L72, S55-L72, D56-L72, S57-L72, H58-L72, S59-L72, L60-L72, S61-L72, S62-L72, F63-L72, T64-L72, Q65-L72, and/or C66-L72 of SEQ ID NO:24. Polynucleotide sequences encoding these polypeptides are also provided. The present invention also encompasses the use of these N-terminal HGPRBMY3 C-terminal fragment deletion polypeptides as immunogenic and/or antigenic epitopes as described elsewhere herein.

In preferred embodiments, the following C-terminal HGPRBMY3 C-terminal fragment deletion polypeptides are encompassed by the present invention: A1-L72, A1-A71, A1-S70, A1-D69, A1-Q68, A1-P67, A1-C66, A1-Q65, A1-T64, A1-F63, A1-S62, A1-S61, A1-L60, A1-S59, A1-H58, A1-S57, A1-D56, A1-S55, A1-P54, A1-R53, A1-L52, A1-L51, A1-G50, A1-Q49, A1-S48, A1-A47, A1-A46, A1-D45, A1-P44, A1-R43, A1-T42, A1-A41, A1-D40, A1-T39, A1-T38, A1-V37, A1-A36, A1-S35, A1-R34, A1-E33, A1-S32, A1-Q31, A1-A30, A1-L29, A1-A28, A1-A27, A1-R26, A1-T25, A1-G24, A1-N23, A1-T22, A1-A21, A1-S20, A1-T19, A1-R18, A1-A17, A1-R16, A1-H15, A1-P14, A1-T13, A1-G12, A1-L11, A1-G10, A1-R9, A1-L8, and/or A1-T7 of SEQ ID NO:24. Polynucleotide sequences encoding these polypeptides are also provided. The present invention also encompasses the use of these C-terminal HGPRBMY3 C-terminal fragment deletion polypeptides as immunogenic and/or antigenic epitopes as described elsewhere herein.

The HGPRBMY3 polypeptides of the present invention were determined to comprise several phosphorylation sites based upon the Motif algorithm (Genetics Computer Group, Inc.). The phosphorylation of such sites may regulate some biological activity of the HGPRBMY3 polypeptide. For example, phosphorylation at specific sites may be involved in regulating the proteins ability to associate or bind to other molecules (e.g., proteins, ligands, substrates, DNA, etc.). In the present case, phosphorylation may modulate the ability of the HGPRBMY3 polypeptide to associate with other polypeptides, particularly cognate ligand for HGPRBMY3, or its ability to modulate certain cellular signal pathways.

The HGPRBMY3 polypeptide was predicted to comprise six PKC phosphorylation site using the Motif algorithm (Genetics Computer Group, Inc.). In vivo, protein kinase C exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget J. R., Gould K. L., Hunter T., Eur. J. Biochem. 161:177-184(1986), and Kishimoto A., Nishiyama K., Nakanishi H., Uratsuji Y., Nomura H., Takeyama Y., Nishizuka Y., J. Biol. Chem. 260:12492-12499(1985); which are hereby incorporated by reference herein.

In preferred embodiments, the following PKC phosphorylation site polypeptides are encompassed by the present invention: PDYRPTHRLHLVV (SEQ ID NO:47), AVVYSSGRVFWTL (SEQ ID NO:48), PDATQSQRRRKTV (SEQ ID NO:49), QRRRKTVRLLLAN (SEQ ID NO:50), EGFRNTLRGLGTP (SEQ ID NO:51), and/or AALAQSERSAVTT (SEQ ID NO:52). Polynucleotides encoding these polypeptides are also provided. The present invention also encompasses the use of the HGPRBMY3 PKC phosphorylation site polypeptides as immunogenic and/or antigenic epitopes as described elsewhere herein.

The HGPRBMY3 polypeptide was predicted to comprise two casein kinase II phosphorylation sites using the Motif algorithm (Genetics Computer Group, Inc.). Casein kinase II (CK-2) is a protein serine/threonine kinase whose activity is independent of cyclic nucleotides and calcium. CK-2 phosphorylates many different proteins. The substrate specificity [1] of this enzyme can be summarized as follows: (1) Under comparable conditions Ser is favored over Thr.; (2) An acidic residue (either Asp or Glu) must be present three residues from the C-terminal of the phosphate acceptor site; (3) Additional acidic residues in positions +1, +2, +4, and +5 increase the phosphorylation rate. Most physiological substrates have at least one acidic residue in these positions; (4) Asp is preferred to Glu as the provider of acidic determinants; and (5) A basic residue at the N-terminal of the acceptor site decreases the phosphorylation rate, while an acidic one will increase it.

A consensus pattern for casein kinase II phosphorylations site is as follows: [ST]-x(2)-[DE], wherein ‘x’ represents any amino acid, and S or T is the phosphorylation site.

Additional information specific to casein kinase II phosphorylation site domains may be found in reference to the following publication: Pinna L. A., Biochim. Biophys. Acta 1054:267-284(1990); which is hereby incorporated herein in its entirety.

In preferred embodiments, the following casein kinase II phosphorylation site polypeptide is encompassed by the present invention: RLCFESFSDELWKG (SEQ ID NO:53), and/or VTTDATRPDAASQG (SEQ ID NO:54). Polynucleotides encoding these polypeptides are also provided. The present invention also encompasses the use of this casein kinase II phosphorylation site polypeptide as an immunogenic and/or antigenic epitope as described elsewhere herein.

The HGPRBMY3 polypeptide was predicted to comprise one cAMP- and cGMP-dependent protein kinase phosphorylation site using the Motif algorithm (Genetics Computer Group, Inc.). There has been a number of studies relative to the specificity of cAMP- and cGMP-dependent protein kinases. Both types of kinases appear to share a preference for the phosphorylation of serine or threonine residues found close to at least two consecutive N-terminal basic residues.

A consensus pattern for cAMP- and cGMP-dependent protein kinase phosphorylation sites is as follows: [RK](2)-x-[ST], wherein “x” represents any amino acid, and S or T is the phosphorylation site.

Additional information specific to cAMP- and cGMP-dependent protein kinase phosphorylation sites may be found in reference to the following publication: Fremisco J. R., Glass D. B., Krebs E. G, J. Biol. Chem. 255:4240-4245(1980); Glass D. B., Smith S. B., J. Biol. Chem. 258:14797-14803(1983); and Glass D. B., El-Maghrabi M. R., Pilkis S. J., J. Biol. Chem. 261:2987-2993(1986); which is hereby incorporated herein in its entirety.

In preferred embodiments, the following cAMP- and cGMP-dependent protein kinase phosphorylation site polypeptide is encompassed by the present invention: TQSQRRRKTVRLLL (SEQ ID NO:55). Polynucleotides encoding this polypeptide are also provided. The present invention also encompasses the use of this cAMP- and cGMP-dependent protein kinase phosphorylation site polypeptide as an immunogenic and/or antigenic epitope as described elsewhere herein.

The HGPRBMY3 polypeptide has been shown to comprise four glycosylation sites according to the Motif algorithm (Genetics Computer Group, Inc.). As discussed more specifically herein, protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following concensus pattern, N-{P}-[ST]-{P}, wherein N represents the glycosylation site. However, it is well known that that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation may be found in reference to the following publications, which are hereby incorporated by reference herein: Marshall R. D., Annu. Rev. Biochem. 41:673-702(1972); Pless D. D., Lennarz W. J., Proc. Natl. Acad. Sci. U.S.A. 74:134-138(1977); Bause E., Biochem. J. 209:331-336(1983); Gavel Y., von Heijne G., Protein Eng. 3:433-442(1990); and Miletich J. P., Broze G. J. Jr., J. Biol. Chem. 265:11397-11404(1990).

In preferred embodiments, the following MLANSSSTNSSV (SEQ ID NO:56), NSSSTNSSVLPCPD (SEQ ID NO:57), CFVPYNSTLAVYGL (SEQ ID NO:58), and/or RTSATNGTRAALAQ (SEQ ID NO:58). Polynucleotides encoding these polypeptides are also provided. The present invention also encompasses the use of these HGPRBMY3 asparagine glycosylation site polypeptide as immunogenic and/or antigenic epitopes as described elsewhere herein.

The HGPRBMY3 polypeptide was predicted to comprise three N-myristoylation sites using the Motif algorithm (Genetics Computer Group, Inc.). An appreciable number of eukaryotic proteins are acylated by the covalent addition of myristate (a C14-saturated fatty acid) to their N-terminal residue via an amide linkage. The sequence specificity of the enzyme responsible for this modification, myristoyl CoA:protein N-myristoyl transferase (NMT), has been derived from the sequence of known N-myristoylated proteins and from studies using synthetic peptides. The specificity seems to be the following: i.) The N-terminal residue must be glycine; ii.) In position 2, uncharged residues are allowed; iii.) Charged residues, proline and large hydrophobic residues are not allowed; iv.) In positions 3 and 4, most, if not all, residues are allowed; v.) In position 5, small uncharged residues are allowed (Ala, Ser, Thr, Cys, Asn and Gly). Serine is favored; and vi.) In position 6, proline is not allowed.

A consensus pattern for N-myristoylation is as follows: G-{EDRKHPFYW}-x(2)-[STAGCN]-{P}, wherein ‘x’ represents any amino acid, and G is the N-myristoylation site.

Additional information specific to N-myristoylation sites may be found in reference to the following publication: Towler D. A., Gordon J. I., Adams S. P., Glaser L., Annu. Rev. Biochem. 57:69-99(1988); and Grand R. J. A., Biochem. J. 258:625-638(1989); which is hereby incorporated herein in its entirety.

In preferred embodiments, the following N-myristoylation site polypeptides are encompassed by the present invention: LVLAAGLPLNALALWV (SEQ ID NO:60), TLAVYGLLRSKLVAAS (SEQ ID NO:61), and/or TSATNGTRAALAQSER (SEQ ID NO:62). Polynucleotides encoding these polypeptides are also provided. The present invention also encompasses the use of these N-myristoylation site polypeptides as immunogenic and/or antigenic epitopes as described elsewhere herein.

Moreover, in confirmation of HGPRBMY3 representing a novel GPCR, the HGPRBMY3 polypeptide was predicted to comprise a G-protein coupled receptor motif using the Motif algorithm (Genetics Computer Group, Inc.). G-protein coupled receptors (also called R7G) are an extensive group of hormones, neurotransmitters, odorants and light receptors which transduce extracellular signals by interaction with guanine nucleotide-binding (G) proteins. Some examples of receptors that belong to this family are provided as follows: 5-hydroxytryptamine (serotonin) 1A to 1F, 2A to 2C, 4, 5A, 5B, 6 and 7, Acetylcholine, muscarinic-type, M1 to M5, Adenosine A1, A2A, A2B and A3, Adrenergic alpha-1A to -1C; alpha-2A to -2D; beta-1 to -3, Angiotensin II types I and II, Bombesin subtypes 3 and 4, Bradykinin B1 and B2, c3a and C5a anaphylatoxin, Cannabinoid CB1 and CB2, Chemokines C-C CC-CKR-1 to CC-CKR-8, Chemokines C-X-C CXC-CKR-1 to CXC-CKR-4, Cholecystokinin-A and cholecystokinin-B/gastrin, Dopamine D1 to D5, Endothelin ET-a and ET-b, fMet-Leu-Phe (fMLP) (N-formyl peptide), Follicle stimulating hormone (FSH-R), Galanin, Gastrin-releasing peptide (GRP-R), Gonadotropin-releasing hormone (GNRH-R), Histamine H1 and H2 (gastric receptor I), Lutropin-choriogonadotropic hormone (LSH-R), Melanocortin MC1R to MC5R, Melatonin, Neuromedin B (NMB-R), Neuromedin K (NK-3R), Neuropeptide Y types 1 to 6, Neurotensin (NT-R), Octopamine (tyramine) from insects, Odorants, Opioids delta-, kappa- and mu-types, Oxytocin (OT-R), Platelet activating factor (PAF-R), Prostacyclin, Prostaglandin D2, Prostaglandin E2, EP1 to EP4 subtypes, Prostaglandin F2, Purinoreceptors (ATP), Somatostatin types 1 to 5, Substance-K (NK-2R), Substance-P (NK-1R), Thrombin, Thromboxane A2, Thyrotropin (TSH-R), Thyrotropin releasing factor (TRH-R), Vasopressin V1a, V1b and V2, Visual pigments (opsins and rhodopsin), Proto-oncogene mas, Caenorhabditis elegans putative receptors C06G4.5, C38C10.1, C43C3.2, T27D1.3 and ZC84.4. Three putative receptors encoded in the genome of cytomegalovirus: US27, US28, and UL33., ECRF3, a putative receptor encoded in the genome of herpesvirus saimiri.

The structure of all GPCRs are thought to be identical. They have seven hydrophobic regions, each of which most probably spans the membrane. The N-terminus is located on the extracellular side of the membrane and is often glycosylated, while the C-terminus is cytoplasmic and generally phosphorylated. Three extracellular loops alternate with three intracellular loops to link the seven transmembrane regions. Most, but not all of these receptors, lack a signal peptide. The most conserved parts of these proteins are the transmembrane regions and the first two cytoplasmic loops. A conserved acidic-Arg-aromatic triplet is present in the N-terminal extremity of the second cytoplasmic loop and could be implicated in the interaction with G proteins.

The putative concensus sequence for GPCRs comprises the conserved triplet and also spans the major part of the third transmembrane helix, and is as follows: [GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R-[FYWCSH]-x(2)-[LIVM], where “X” represents any amino acid.

Additional information relating to G-protein coupled receptors may be found in reference to the following publications: Strosberg A. D., Eur. J. Biochem. 196:1-10(1991); Kerlavage A. R., Curr. Opin. Struct. Biol. 1:394-401(1991); Probst W. C., Snyder L. A., Schuster D. I., Brosius J., Sealfon S. C., DNA Cell Biol. 11: 1-20(1992); Savarese T. M., Fraser C. M., Biochem. J. 283:1-9(1992); Branchek T., Curr. Biol. 3:315-317(1993); Stiles G. L., J. Biol. Chem. 267:6451-6454(1992); Friell T., Kobilka B. K., Lefkowitz R. J., Caron M. G., Trends Neurosci. 11:321-324(1988); Stevens C. F., Curr. Biol. 1:20-22(1991); Sakurai T., Yanagisawa M., Masaki T., Trends Pharmacol. Sci. 13:103-107(1992); Salesse R., Remy J. J., Levin J. M., Jallal B., Gamier J., Biochimie 73:109-120(1991); Lancet D., Ben-Arie N., Curr. Biol. 3:668-674(1993); Uhl G. R., Childers S., Pasternak G., Trends Neurosci. 17:89-93(1994); Barnard E. A., Burnstock G., Webb T. E., Trends Pharmacol. Sci. 15:67-70(1994); Applebury M. L., Hargrave P. A., Vision Res. 26:1881-1895(1986); Attwood T. K., Eliopoulos E. E., Findlay J. B. C., Gene 98:153-159(1991); http://www.gcrdb.uthscsa.edu/; and http://swift.embl-heidelberg.de/7tm/.

In preferred embodiments, the following G-protein coupled receptors signature polypeptide is encompassed by the present invention: QMNMYGSCIFLMLINVDRYAAIVHPLR (SEQ ID NO:63). Polynucleotides encoding this polypeptide is also provided. The present invention also encompasses the use of the HGPRBMY3 G-protein coupled receptors signature polypeptide as immunogenic and/or antigenic epitopes as described elsewhere herein.

For the production of antibodies, various hosts including goats, rabbits, sheep, rats, mice, humans, and others, can be immunized by injection with HGPRBMY3 polypeptide, or any fragment or oligopeptide thereof, which has immunogenic properties. Depending on the host species, various adjuvants may be used to increase the immunological response. Non-limiting examples of suitable adjuvants include Freund's (incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guérin) and Corynebacterium parvumn.

Preferably, the peptides, fragments, or oligopeptides used to induce antibodies to HGPRBMY3 polypeptide (i.e., immunogens) have an amino acid sequence having at least five amino acids, and more preferably, at least 7-10 amino acids. It is also preferable that the immunogens are identical to a portion of the amino acid sequence of the natural protein; they may also contain the entire amino acid sequence of a small, naturally occurring molecule. The peptides, fragments or oligopeptides may comprise a single epitope or antigenic determinant or multiple epitopes. Short stretches of HGPRBMY3 amino acids may be fused with those of another protein, such as KLH, and antibodies are produced against the chimeric molecule.

Monoclonal antibodies to HGPRBMY3 polypeptide, or immunogenic fragments thereof may be prepared using any technique which provides for the production of antibody molecules by continuous cell lines in culture. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-42; R. J. Cote et al., 1983, Proc. Natl. Acad. Sci. USA, 80:2026-2030; and S. P. Cole et al., 1984, Mol. Cell Biol., 62:109-120). The production of monoclonal antibodies is well known and routinely used in the art.

In addition, techniques developed for the production of “chimeric antibodies,” the splicing of mouse antibody genes to human antibody genes to obtain a molecule with appropriate antigen specificity and biological activity can be used (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques described for the production of single chain antibodies may be adapted, using methods known in the art, to produce HGPRBMY3 polypeptide-specific single chain antibodies. Antibodies with related specificity, but of distinct idiotypic composition, may be generated by chain shuffling from random combinatorial immunoglobulin libraries (D. R. Burton, 1991, Proc. Natl. Acad. Sci. USA, 88:11120-3). Antibodies may also be produced by inducing in vivo production in the lymphocyte population or by screening recombinant immunoglobulin libraries or panels of highly specific binding reagents as disclosed in the literature (R. Orlandi et al., 1989, Proc. Natl.. Acad. Sci. USA, 86:3833-3837 and G. Winter et al., 1991, Nature, 349:293-299).

Antibody fragments, which contain specific binding sites for HGPRBMY3 polypeptide, may also be generated. For example, such fragments include, but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed to allow rapid and easy identification of monoclonal Fab fragments with the desired specificity (W. D. Huse et al., 1989, Science, 254.1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between HGPRBMY3 polypeptide and its specific antibody. A two-site, monoclonal-based immunoassay utilizing monoclonal antibodies reactive with two non-interfering HGPRBMY3 polypeptide epitopes is preferred, but a competitive binding assay may also be employed (Maddox, supra).

Another aspect of the invention relates to a method for inducing an immunological response in a mammal which comprises inoculating the mammal with HGPRBMY3 polypeptide, or a fragment thereof, adequate to produce antibody and/or T cell immune response to protect said animal from infections such as bacterial, fungal, protozoan and viral infections, particularly infections caused by HIV-1 or HIV-2. Yet another aspect of the invention relates to a method of inducing immunological response in a mammal which comprises, delivering HGPRBMY3 polypeptide via a vector directing expression of HGPRBMY3 polynucleotide in vivo in order to induce such an immunological response to produce antibody to protect said animal from diseases.

A further aspect of the invention relates to an immunological/vaccine formulation (composition) which, when introduced into a mammalian host, induces an immunological response in that mammal to an HGPRBMY3 polypeptide wherein the composition comprises an HGPRBMY3 polypeptide or HGPRBMY3 gene. The vaccine formulation may further comprise a suitable carrier. Since the HGPRBMY3 polypeptide may be broken down in the stomach, it is preferably administered parenterally (including subcutaneous, intramuscular, intravenous, intradermal, etc., injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials, and may be stored in a freeze-dried condition requiring only the addition of the sterile liquid carrier immediately prior to use. The vaccine formulation may also include adjuvant systems for enhancing the immunogenicity of the formulation, such as oil-in-water systems and other systems known in the art. The dosage will depend on the specific activity of the vaccine and can be readily determined by routine experimentation.

In an embodiment of the present invention, the polynucleotide encoding the HGPRBMY3 polypeptide, or any fragment or complement thereof, may be used for therapeutic purposes. In one aspect, antisense, to the polynucleotide encoding the HGPRBMY3 polypeptide, may be used in situations in which it would be desirable to block the transcription of the mRNA. In particular, cells may be transformed with sequences complementary to polynucleotides encoding HGPRBMY3 polypeptide. Thus, complementary molecules may be used to modulate HGPRBMY3 polynucleotide and polypeptide activity, or to achieve regulation of gene function. Such technology is now well known in the art, and sense or antisense oligomers or oligonucleotides, or larger fragments, can be designed from various locations along the coding or control regions of polynucleotide sequences encoding HGPRBMY3 polypeptide.

Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors which will express a nucleic acid sequence that is complementary to the nucleic acid sequence encoding the HGPRBMY3 polypeptide. These techniques are described both in J. Sambrook et al., supra and in F. M. Ausubel et al., supra.

Polypeptides used in treatment can also be generated endogenously in the subject, in treatment modalities often referred to as “gene therapy”. Thus for example, cells from a subject may be engineered with a polynucleotide, such as DNA or RNA, to encode a polypeptide ex vivo, and for example, by the use of a retroviral plasmid vector. The cells can then be introduced into the subject.

The genes encoding the HGPRBMY3 polypeptide can be turned off by transforming a cell or tissue with an expression vector that expresses high levels of an HGPRBMY3 polypeptide-encoding polynucleotide, or a fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and even longer if appropriate replication elements are designed to be part of the vector system.

Modifications of gene expression can be obtained by designing antisense molecules or complementary nucleic acid sequences (DNA, RNA, or PNA), to the control, 5′, or regulatory regions of the gene encoding the HGPRBMY3 polypeptide, (e.g., signal sequence, promoters, enhancers, and introns). Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (see, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence may also be designed to block translation of mRNA by preventing the transcript from binding to ribosomes.

Ribozymes, i.e., enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Suitable examples include engineered hammerhead motif ribozyme molecules that can specifically and efficiently catalyze endonucleolytic cleavage of sequences encoding HGPRBMY3 polypeptide.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes according to the invention may be prepared by any method known in the art for the synthesis of nucleic acid molecules. Such methods include techniques for chemically synthesizing oligonucleotides, for example, solid phase phosphoramidite chemical synthesis. Alternatively, RNA molecules may be generated by in vitro and in vivo transcription of DNA sequences encoding HGPRBMY3. Such DNA sequences may be incorporated into a wide variety of vectors with suitable RNA polymerase promoters such as T7 or SP. Alternatively, the cDNA constructs that constitutively or inducibly synthesize complementary RNA can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl, rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytosine, guanine, thymine, and uridine which are not as easily recognized by endogenous endonucleases.

Many methods for introducing vectors into cells or tissues are available and are equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art.

Any of the therapeutic methods described above may be applied to any individual in need of such therapy, including, for example, mammals such as dogs, cats, cows, horses, rabbits, monkeys, and most preferably, humans.

A further embodiment of the present invention embraces the administration of a pharmaceutical composition, in conjunction with a pharmaceutically acceptable carrier, diluent, or excipient, for any of the above-described therapeutic uses and effects. Such pharmaceutical compositions may comprise HGPRBMY3 nucleic acid, polypeptide, or peptides, antibodies to HGPRBMY3 polypeptide, mimetics, agonists, antagonists, or inhibitors of HGPRBMY3 polypeptide or polynucleotide. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (i.e., the HGPRBMY3 nucleic acid or polypeptide, or functional fragments thereof), the pharmaceutical compositions may contain suitable pharmaceutically acceptable carriers or excipients comprising auxiliaries which facilitate processing of the active compounds into preparations which can be used pharmaceutically. Further details on techniques for formulation and administration are provided in the latest edition of Remington 's Pharmaceutical Sciences (Mack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which can be used orally, include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants or permeation agents that are appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of HGPRBMY3 product, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, e.g., using neoplastic cells, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, HGPRBMY3 polypeptide, or fragments thereof, antibodies to HGPRBMY3 polypeptide, agonists, antagonists or inhibitors of HGPRBMY3 polypeptide, which ameliorates, reduces, or eliminates the symptoms or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, ED₅₀/LD₅₀. Pharmaceutical compositions, which exhibit large therapeutic indices, are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, sensitivity of the patient, and the route of administration.

The exact dosage will be determined by the practitioner, who will consider the factors related to the individual requiring treatment. Dosage and administration are adjusted to provide sufficient levels of the active moiety or to maintain the desired effect. Factors, which may be taken into account, include the severity of the individual's disease state, general health of the patient, age, weight, and gender of the patient, diet, time and frequency of administration, drug combination(s), reaction sensitivities, and tolerance/response to therapy. As a general guide, long-acting pharmaceutical compositions may be administered every 3 to 4 days, every week, or once every two weeks, depending on half-life and clearance rate of the particular formulation. Variations in these dosage levels can be adjusted using standard empirical routines for optimization, as is well understood in the art.

Normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors. Similarly, delivery of polynucleotides or polypeptides will be specific to particular cells, conditions, locations, and the like.

In another embodiment of the present invention, antibodies which specifically bind to the HGPRBMY3 polypeptide may be used for the diagnosis of conditions or diseases characterized by expression (or overexpression) of the HGPRBMY3 polynucleotide or polypeptide, or in assays to monitor patients being treated with the HGPRBMY3 polypeptide, or its agonists, antagonists, or inhibitors. The antibodies useful for diagnostic purposes may be prepared in the same manner as those described herein for use in therapeutic methods. Diagnostic assays for the HGPRBMY3 polypeptide include methods, which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules, which are known in the art, may be used, several of which are described above.

The use of mammalian cell reporter assays to demonstrate functional coupling of known GPCRs (G Protein Coupled Receptors) has been well documented in the literature (Gilman, 1987, Boss et al., 1996; Alam & Cook, 1990; George et al., 1997; Selbie & Hill, 1998; Rees et al., 1999). In fact, reporter assays have been successfully used for identifying novel small molecule agonists or antagonists against GPCRs as a class of drug targets (7, 5; 4; Rees et al, 2001). In such reporter assays, a promoter is regulated as a direct consequence of activation of specific signal transduction cascades following agonist binding to a GPCR (Alam & Cook 1990; Selbie & Hill, 1998; Boss et al., 1996; 5; Gilman, 1987).

A number of response element-based reporter systems have been developed that enable the study of GPCR function. These include cAMP response element (CRE)-based reporter genes for G alpha i/o, G alpha s-coupled GPCRs, Nuclear Factor Activator of Transcription (NFAT)-based reporters for G alpha q/l 1 or the promiscuous G protein G alpha 15/16-coupled receptors and MAP kinase reporter genes for use in Galpha i/o coupled receptors (Selbie & Hill, 1998; Boss et al., 1996; 5; Blahos, et al., 2001; Offernann & Simon, 1995; Gilman, 1987; Rees et al., 2001). Transcriptional response elements that regulate the expression of Beta-Lactamase within a CHO K1 cell line (CHO-NFAT/CRE: Aurora Biosciences™) (7) have been implemented to characterize the function of the orphan HGPRBMY3 polypeptide of the present invention. The system enables demonstration of constitutive G-protein coupling to endogenous cellular signaling components upon intracellular overexpression of orphan receptors. Overexpression has been shown to represent a physiologically relevant event. For example, it has been shown that overexpression occurs in nature during metastatic carcinomas, wherein defective expression of the monocyte chemotactic protein 1 receptor, CCR2, in macrophages is associated with the incidence of human ovarian carcinoma (14, 13). Indeed, it has been shown that overproduction of the Beta 2 Adrenergic Receptor in transgenic mice leads to constitutive activation of the receptor signaling pathway such that these mice exhibit increased cardiac output (15; 16). These are only a few of the many examples demonstrating constitutive activation of GPCRs whereby many of these receptors are likely to be in the active, R*, conformation (17) (see Example 8).

Several assay protocols including ELISA, RIA, and FACS for measuring HGPRBMY3 polypeptide are known in the art and provide a basis for diagnosing altered or abnormal levels of HGPRBMY3 polypeptide expression. Normal or standard values for HGPRBMY3 polypeptide expression are established by combining body fluids or cell extracts taken from normal mammalian subjects, preferably human, with antibody to the HGPRBMY3 polypeptide under conditions suitable for complex formation. The amount of standard complex formation may be quantified by various methods; photometric means are preferred. Quantities of HGPRBMY3 polypeptide expressed in subject sample, control sample, and disease samples from biopsied tissues are compared with the standard values. Deviation between standard and subject values establishes the parameters for diagnosing disease.

Microarrays and Screening Assays

In another embodiment of the present invention, oligonucleotides, or longer fragments derived from the HGPRBMY3 polynucleotide sequence described herein may be used as targets in a microarray. The microarray can be used to monitor the expression level of large numbers of genes simultaneously (to produce a transcript image), and to identify genetic variants, mutations and polymorphisms. This information may be used to determine gene function, to understand the genetic basis of a disease, to diagnose disease, and to develop and monitor the activities of therapeutic agents. In a particular aspect, the microarray is prepared and used according to the methods described in WO 95/11995 (Chee et al.); D. J. Lockhart et al., 1996, Nature Biotechnology, 14:1675-1680; and M. Schena et al., 1996, Proc. Natl.. Acad. Sci. USA, 93:10614-10619). Microarrays are further described in U.S. Pat. No. 6,015,702 to P. Lal et al.

In another embodiment of this invention, the nucleic acid sequence, which encodes the HGPRBMY3 polypeptide, may also be used to generate hybridization probes, which are useful for mapping the naturally occurring genomic sequence. The sequences may be mapped to a particular chromosome, to a specific region of a chromosome, or to artificial chromosome constructions (HACs), yeast artificial chromosomes (YACs), bacterial artificial chromosomes (BACs), bacterial PI constructions, or single chromosome cDNA libraries, as reviewed by C. M. Price, 1993, Blood Rev., 7:127-134 and by B. J. Trask, 1991, Trends Genet., 7:149-154.

Fluorescent In Situ Hybridization (FISH), (as described in I. Verma et al., 1988, Human Chromosomes: A Manual of Basic Techniques Pergamon Press, New York, N.Y.) may be correlated with other physical chromosome mapping techniques and genetic map data. Examples of genetic map data can be found in numerous scientific journals or at Online Mendelian Inheritance in Man (OMIM). Correlation between the location of the gene encoding the HGPRBMY3 polypeptide on a physical chromosomal map and a specific disease, or predisposition to a specific disease, may help delimit the region of DNA associated with that genetic disease. The nucleotide sequences, particularly that of SEQ ID NO: 1, or fragments thereof, according to this invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers, even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (R. A. Gatti et al., 1988, Nature, 336:577-580), any sequences mapping to that area may represent associated or regulatory genes for further investigation. The nucleotide sequence of the present invention may also be used to detect differences in the chromosomal location due to translocation, inversion, and the like, among normal, carrier, or affected individuals.

In another embodiment of the present invention, the HGPRBMY3 polypeptide, its catalytic or immunogenic fragments or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between HGPRBMY3 polypeptide, or portion thereof, and the agent being tested, may be measured utilizing techniques commonly practiced in the art.

Another technique for drug screening, which may be used, provides for high throughput screening of compounds having suitable binding affinity to the protein of interest as described in WO 84/03564 (Venton, et al.). In this method, as applied to the HGPRBMY3 protein, large numbers of different small test compounds are synthesized on a solid substrate, such as plastic pins or some other surface. The test compounds are reacted with the HGPRBMY3 polypeptide or fragments thereof, and washed. Bound HGPRBMY3 polypeptide is then detected by methods well known in the art. Purified HGPRBMY3 polypeptide can also be coated directly onto plates for use in the aforementioned drug screening techniques. Alternatively, non-neutralizing antibodies can be used to capture the peptide and immobilize it on a solid support.

In a further embodiment of this invention, competitive drug screening assays can be used in which neutralizing antibodies, capable of binding the HGPRBMY3 polypeptide, specifically compete with a test compound for binding to the HGPRBMY3 polypeptide. In this manner, the antibodies can be used to detect the presence of any peptide, which shares one or more antigenic determinants with the HGPRBMY3 polypeptide.

### EXAMPLES

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publication's, for example, Sambrook, Fritsch, and Maniatis, Molecular Cloning: a Laboratory Manual, 2^{nd} Edition, Cold Spring Harbor Laboratory Press, USA, (1989).

### Example 1

### Bioinformatics Analysis

G-protein coupled receptor sequences were used as a probe to search the Incyte and public domain EST databases. The search program used was gapped BLAST (S. F. Altschul, et al., Nuc. Acids Res., 25:3389-4302 (1997)). The top EST hits from the BLAST results were searched back against the non-redundant protein and patent sequence databases. From this analysis, ESTs encoding potential novel GPCRs were identified based on sequence homology. The Incyte EST (CloneID:3356166) was selected as potential novel GPCR candidate, called HGPRBMY3, for subsequent analysis. This EST was sequenced and the full-length clone of this GPCR was obtained using the EST sequence information and conventional methods. The complete protein sequence of HGPRBMY3 was analyzed for potential transmembrane domains. The TMPRED program (K. Hofmann and W. Stoffel, Biol. Chem., 347:166 (1993)) was used for transmembrane prediction. The program predicted seven transmembrane domains and the predicted domains match with the predicated transmembrane domains of related GPCRs at the sequence level. Based on sequence, structure and known GPCR signature sequences, the orphan protein, HGPRBMY3, is a novel human GPCR.

### Example 2

### Cloning of the Novel Human GPCR HGPRBMY3

Using the EST sequence, an antisense 80 base pair oligonucleotide with biotin on the 5′ end was designed that was complementary to the putative coding region of HGPRBMY3 as follows: 5′-b-CCA AGC TGT AGA CCA CCA AGT GCA GGC GGT GGG TAG GTC GGT AGT CAG GAC ACG GGA GAA CAG AAC TGT TGG TTG AGG AG-3′ (SEQ ID NO:5). This biotinylated oligo was incubated with a mixture of single-stranded covalently closed circular cDNA libraries, which contained DNA corresponding to the sense strand. Hybrids between the biotinylated oligo and the circular cDNA were captured on streptavidin magnetic beads. Upon thermal release of the cDNA from the biotinylated oligo, the single stranded cDNA was converted into double strands using a primer homologous to a sequence on the cDNA cloning vector. The double stranded cDNA was introduced into E. coli by electroporation and the resulting colonies were screened by PCR, using a primer pair designed from the EST sequence to identify the proper cDNA.

Oligonucleotides used to identify the cDNA by PCR were as follows: HGPRBMY3s (SEQ ID NO:6) 5′-AGCCCAATGG CAGACTTGAG-3′; and HGPRBMY3a (SEQ ID NO:7) 5′-GGTGCGTTTG GTCACAGCTT-3′

Those cDNA clones that were positive by PCR had the inserts sized and two of the largest clones (7.0 Kb and 3.0 Kb) were chosen for DNA sequencing. Both clones had identical sequence over the common regions.

### Example 3

### Expression Profiling of Novel Human GPCR, HGPRBMY3

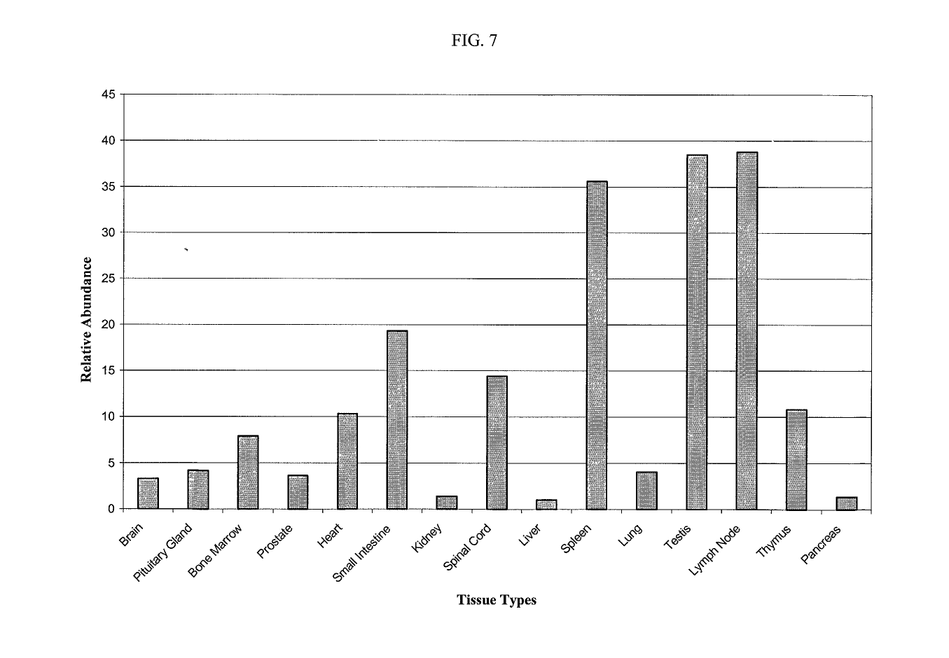

The same PCR primer pair used to identify HGPRBMY3 cDNA clones (HGPRBMY3s-SEQ ID NO:6 and HGPRBMY3a-SEQ ID NO:7) was used to measure the steady state levels of mRNA by quantitative PCR. Briefly, first strand cDNA was made from commercially available mRNA. The relative amount of cDNA used in each assay was determined by performing a parallel experiment using a primer pair for the cyclophilin gene, which is expressed in equal amounts in all tissues. The cyclophilin primer pair detected small variations in the amount of cDNA in each sample, and these data were used for normalization of the data obtained with the primer pair for HGPRBMY3. The PCR data were converted into a relative assessment of the difference in transcript abundance among the tissues tested and the data are presented in FIG. 7. Transcripts corresponding to the orphan GPCR, HGPRBMY3, were found to be highly expressed in immune- and testes-related tissues.

### Example 4

### G-Protein Coupled Receptor Immunohistochemistry Hybridization Expression Profiling

Immunohistochemistry expression using the LifeSpan database, describes positive staining in tumor cells from ovarian carcinoma, colonic adenocarcinoma, pancreatic carcinoma, lung adenocarcinoma, breast carcinoma, and melanoma. Slides containing paraffin sections (LifeSpan BioSciences, Inc.; Seattle, Wash.) were deparaffinized through xylene and alcohol, rehydrated, and then subjected to the steam method of target retrieval (#S1700; DAKO Corp.; Carpenteria, Calif.).

Immunohistochemical assay techniques are commonly known in the art and are described briefly herein. Immunocytochemical (ICC) experiments were performed on a DAKO autostainer following the procedures and reagents developed by DAKO. Specifically, the slides were blocked with avidin, rinsed, blocked with biotin, rinsed, protein blocked with DAKO universal protein block, machine blown dry, primary antibody, incubated, and the slides rinsed. Biotinylated secondary antibody was applied using the manufacturer's instructions (1 drop/10 ml, or approximately 0.75 μg/mL), incubated, rinsed slides, and applied Vectastain ABC-AP reagent for 30 minutes. Vector Red was used as substrate and prepared according to the manufacturer's instructions just prior to use.

Strong positivity was identified in the adrenal medulla. Blush staining was present in the zona glomerulosa of the cortex, but the zonae fasciculata and reticularis were negative. In the surrounding soft tissue, ganglion cells showed blush staining, but Schwann cells were negative. Adipocytes and fibroblasts were also negative.

In the central nervous system, the most prominent positivity was identified in neurons of the lateral mammillary and tuberomammillary nuclei of the hypothalamus, in a subset of cells in the anterior pituitary, and in Herring bodies in the posterior pituitary. Less prominent positivity was identified in pituicytes of the posterior pituitary, and Rathke's pouch remnants of the pars intermedia were negative. In the hippocampus, prominent neurophil positivity was seen in a distribution consistent with mossy fibers originating from the granular neurons of the dentate gyrus. Faint positivity was identified in the cell bodies of the adjacent granular neurons, and occasionally in the pyramidal neurons of areas CA2 through CA4. Other areas showing focal neuronal positivity included the cortex, substantia nigra, diagonal band of Broca, paraventricular nucleus of the hypothalamus, cerebellar granular neurons, and rare Golgi neurons. Blush or faint staining was identified and in various medullary nuclei, and in neurons in the amygdala. Negative neuronal groups included those of the caudate, putamen, thalamus, and various hypothalamic nuclei (other than those already mentioned). A few areas, such as the caudate and putamen, showed blush synaptic labeling surrounding negative neurons. Astrocytes were often positive with this antibody, but oligodendrocytes and microglia were negative.

Outside of the central nervous system the most prominent positivity was identified in the adrenal medulla, adrenal zona glomerulosa, neutrophils, spermatocytic precursors, ovarian mesothelium (germinal epithelium), endometrium, and hepatocytes. Prominent positivity was also identified in neuroendocrine cells in the small and large intestinal epithelium, renal collecting ducts, respiratory epithelium, occasional macrophages, breast epithelium, and prostatic epithelium. Interestingly, ganglion cells and Schwann cells were strongly positive in periadrenal soft tissue, but were negative or showed only faint staining in other areas. Sweat lobules were strongly positive, but ducts were largely negative. Gastric parietal cells were positive in one sample and negative in the second. In the thyroid, rare parafollicular cells were faintly positive, but follicular epithelium was negative.

Other faintly positive cell types included skeletal muscle, visceral smooth muscle, vascular smooth muscle, distal convoluted tubules, oocytes, gastric chief cells, and pneumocytes. Blush staining was identified in urothelium, squamous epithelium, and rarely in endothelium and renal thin loops of Henle. Negative tissues and cell types included Leydig cells, fibroblasts, epididymis, adrenal zona fasciculata, zona reticularis, adipocytes, prostatic fibromuscular stroma, and lymphocytes.

Because of slight variation in staining between antibodies 22315-217 and 22315-330, sections of several peripheral tissues were examined with a third antibody (22315-155). Briefly, the 22315-217 antibody was raised against the following HGPRBMY3 peptide sequence: TLARPDATQSQRRRKTVRL (SEQ ID NO:25); the 22315-330 antibody was raised in rabbits against the following HPRBMY3 peptide sequence: AQSERSAVTTDATRPD (SEQ ID NO:26); and the 22315-155 antibody was raised in rabbits against the following HGPRBMY3 peptide sequence: PAARVHRPSRCRYRD (SEQ ID NO:27). In general, the third antibody supported the staining characteristics identified with antibody 22315-217. Specifically, positivity was identified in pancreatic islets, spermatocytic precursors, prostatic epithelium, sweat lobules, endometrium, neutrophils, and focally in gastric parietal cells.

In tumors, clusters (subpopulations) of intensely positive tumor cells were identified in many of the samples. This included ovarian carcinoma, colonic adenocarcinoma, pancreatic carcinoma, lung adenocarcinoma, breast carcinoma, and melanoma. Prostatic adenocarcinoma and small cell carcinoma were largely negative. The staining in tumors with this antibody varied from that with the second antibody (22315-330). Additional tumor samples will be examined with both antibodies in future studies.

### Example 5

### G-Protein Coupled Receptor PCR Expression Profiling

RNA quantification was performed using the Taqman® real-time-PCR fluorogenic assay. The Taqman® assay is one of the most precise methods for assaying the concentration of nucleic acid templates.

All cell lines were grown using standard conditions: RPMI 1640 supplemented with 10% fetal bovine serum, 100 IU/ml penicillin, 100 mg/ml streptomycin, and 2 mM L-glutamine, 10 mM HEPES (all from GibcoBRL; Rockville, Md.). Eighty percent confluent cells were washed twice with phosphate-buffered saline (GibcoBRL) and harvested using 0.25% trypsin (GibcoBRL). RNA was prepared using the RNeasy Maxi Kit from Qiagen (Valencia, Calif.).

cDNA template for real-time PCR was generated using the Superscript™ First Strand Synthesis system for RT-PCR.

SYBR Green real-time PCR reactions were prepared as follows: The reaction mix consisted of 20 ng first strand cDNA; 50 nM Forward Primer; 50 nM Reverse Primer; 0.75× SYBR Green I (Sigma); 1× SYBR Green PCR Buffer (50 mMTris-HCl pH 8.3, 75 mM KCl); 10% DMSO; 3 mM MgCl₂; 300 μM each dATP, dGTP, dTTP, dCTP; 1 U Platinum® Taq DNA Polymerase High Fidelity (Cat# 11304-029; Life Technologies; Rockville, Md.); 1:50 dilution; ROX (Life Technologies). Real-time PCR was performed using an Applied Biosystems 5700 Sequence Detection System. Conditions were 95° C. for 10 min (denaturation and activation of Platinum® Taq DNA Polymerase), 40 cycles of PCR (95° C. for 15 sec, 60° C. for 1 min). PCR products are analyzed for uniform melting using an analysis algorithm built into the 5700 Sequence Detection System. Forward primer: GPCR19-F2: 5′-CCTGGCTTCCACACTTTGTACTC-3′ (SEQ ID NO:28); and Reverse primer: GPCR19-R2: 5′-TCCCAACGCCTCTCGTTCT-3′ (SEQ ID NO:29).

cDNA quantification used in the normalization of template quantity was performed using Taqman® technology. Taqman® reactions are prepared as follows: The reaction mix consisted of 20 ng first strand cDNA; 25 nM GAPDH-F3, Forward Primer; 250 nM GAPDH-R1 Reverse Primer; 200 nM GAPDH-PVIC Taqman® Probe (fluorescent dye labeled oligonucleotide primer); IX Buffer A (Applied Biosystems); 5.5 mM MgCl12; 300 μM dATP, dGTP, dTTP, dCTP; 1 U Amplitaq Gold (Applied Biosystems). GAPDH, D-glyceraldehyde-3-phosphate dehydrogenase, was used as control to normalize mRNA levels.

Real-time PCR was performed using an Applied Biosystems 7700 Sequence Detection System. Conditions were 95° C. for 10 min. (denaturation and activation of Amplitaq Gold), 40 cycles of PCR (95° C. for 15 sec, 60° C. for 1 min).

The sequences for the GAPDH oligonucleotides used in the Taqman® reactions are as follows:

GAPDH-F3-5′-AGCCGAGCCACATCGCT-3′ (SEQ ID NO:30)

GAPDH-R1-5′-GTGACCAGGCGCCCAATAC-3′ (SEQ ID NO:31)

GAPDH-PVIC Taqman® Probe-VIC-5′-CAAATCCGTTGACTCCGACCTTCACCTT-3′ TAMRA (SEQ ID NO:32).

The Sequence Detection System generates a Ct (threshold cycle) value that is used to calculate a concentration for each input cDNA template. cDNA levels for each gene of interest are normalized to GAPDH cDNA levels to compensate for variations in total cDNA quantity in the input sample. This is done by generating GAPDH Ct values for each cell line. Ct values for the gene of interest and GAPDH are inserted into a modified version of the δδCt equation (Applied Biosystems Prism® 7700 Sequence Detection System User Bulletin #2), which is used to calculate a GAPDH normalized relative cDNA level for each specific cDNA. The δδCt equation is as follows: relative quantity of nucleic acid template=2^{δδCt}=2^{(δCta-δCtb)}, where δCta=Ct target−Ct GAPDH, and δCtb=Ct reference−Ct GAPDH. (No reference cell line was used for the calculation of relative quantity; δCtb was defined as 21).

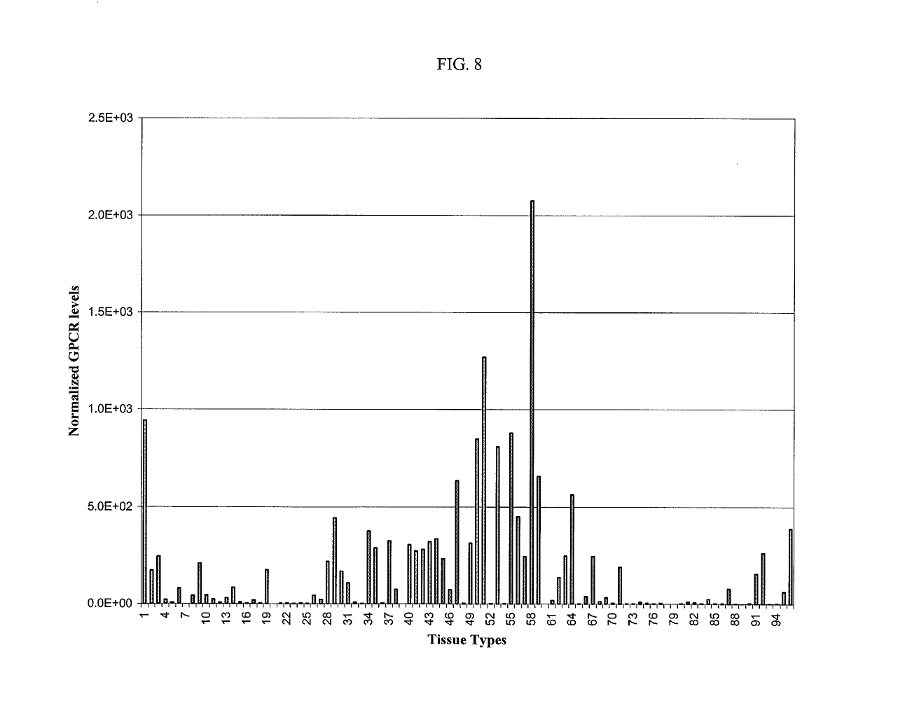

The Graph # of Table 1 corresponds to the tissue type position number of FIG. 8. Interestingly, HGPRBMY3 (also known as GPCRl9 or GPR92) was found to be overexpressed 500 to 2000 fold greater in colon carcinoma cell lines in comparison to other cancer cell lines in the OCLP-1 (oncology cell line panel). Additionally, HGPRBMY3 is sporadically expressed at moderate to high levels in breast and ovarian tumor cell lines. This expression pattern is similar to that seen with the Lifespan analysis of HGPRBMY3 protein in colon, breast and ovarian tumor tissue. The Lifespan data consistently show high expression of HGPRBMY3 protein in colon tumors and sporadically show moderate to high expression of HGPRBMY3 in breast and ovarian tumors.

TABLE 1

| Graph | | | Ct | Ct | | | |
|---|---|---|---|---|---|---|---|
| # | Name | Tissue | GAPDH | GPCR19-2 | dCt | ddCt | Quant. |
| 1 | AIN 4 | breast | 17.49 | 28.61 | 11.12 | −9.88 | 9.4E + 02 |
| 2 | AIN 4T | breast | 17.15 | 30.73 | 13.58 | −7.42 | 1.7E + 02 |
| 3 | AIN4/myc | breast | 17.81 | 30.88 | 13.07 | −7.93 | 2.4E + 02 |
| 4 | BT-20 | breast | 17.9 | 34.62 | 16.72 | −4.28 | 1.9E + 01 |
| 5 | BT-474 | breast | 17.65 | 36.1 | 18.45 | −2.55 | 5.9E + 00 |
| 6 | BT-483 | breast | 17.45 | 32.13 | 14.68 | −6.32 | 8.0E + 01 |
| 7 | BT-549 | breast | 17.55 | 40 | 22.45 | 1.45 | 0.0E + 00 |
| 8 | DU4475 | breast | 18.1 | 33.69 | 15.59 | −5.41 | 4.3E + 01 |
| 9 | H3396 | breast | 18.04 | 31.33 | 13.29 | −7.71 | 2.1E + 02 |
| 10 | HBL100 | breast | 17.02 | 32.57 | 15.55 | −5.45 | 4.4E + 01 |
| 11 | Her2 MCF-7 | breast | 19.26 | 35.79 | 16.53 | −4.47 | 2.2E + 01 |
| 12 | HS 578T | breast | 17.83 | 36.19 | 18.36 | −2.64 | 6.2E + 00 |
| 13 | MCF7 | breast | 17.83 | 33.96 | 16.13 | −4.87 | 2.9E + 01 |
| 14 | MCF-7/AdrR | breast | 17.23 | 31.84 | 14.61 | −6.39 | 8.4E + 01 |
| 15 | MDAH 2774 | breast | 16.87 | 34.95 | 18.08 | −2.92 | 7.6E + 00 |
| 16 | MDA-MB- | breast | 15.72 | 37.19 | 21.47 | 0.47 | 7.2E − 01 |
| | 175-VII | | | | | | |
| 17 | MDA-MB- | breast | 17.62 | 34.49 | 16.87 | −4.13 | 1.8E + 01 |
| | 231 | | | | | | |
| 18 | MDA-MB- | breast | 17.9 | 37.53 | 19.63 | −1.37 | 2.6E + 00 |
| | 453 | | | | | | |
| 19 | MDA-MB- | breast | 17.49 | 31.04 | 13.55 | −7.45 | 1.7E + 02 |
| | 468 | | | | | | |
| 20 | Pat-21 R60 | breast | 35.59 | 40 | 4.41 | −16.59 | ND |
| 21 | SKBR3 | breast | 17.12 | 38.24 | 21.12 | 0.12 | 9.2E − 01 |
| 22 | T47D | breast | 18.86 | 38.12 | 19.26 | −1.74 | 3.3E + 00 |
| 23 | UACC-812 | breast | 17.06 | 39.23 | 22.17 | 1.17 | 4.4E − 01 |
| 24 | ZR-75-1 | breast | 15.95 | 35.4 | 19.45 | −1.55 | 2.9E + 00 |
| 25 | C-33A | cervical | 17.49 | 38.9 | 21.41 | 0.41 | 7.5E − 01 |
| 26 | Ca Ski | cervical | 17.38 | 32.94 | 15.56 | −5.44 | 4.3E + 01 |
| 27 | HeLa | cervical | 17.59 | 34.17 | 16.58 | −4.42 | 2.1E + 01 |
| 28 | HT-3 | cervical | 17.42 | 30.65 | 13.23 | −7.77 | 2.2E + 02 |
| 29 | ME-180 | cervical | 16.86 | 29.07 | 12.21 | −8.79 | 4.4E + 02 |
| 30 | SiHa | cervical | 18.07 | 31.68 | 13.61 | −7.39 | 1.7E + 02 |
| 31 | SW756 | cervical | 15.59 | 29.83 | 14.24 | −6.76 | 1.1E + 02 |
| 32 | CACO-2 | colon | 17.56 | 35.79 | 18.23 | −2.77 | 6.8E + 00 |
| 33 | CCD-112Co | colon | 18.03 | 39.19 | 21.16 | 0.16 | 9.0E − 01 |
| 34 | CCD-33Co | colon | 17.07 | 29.52 | 12.45 | −8.55 | 3.7E + 02 |
| 35 | Colo 205 | colon | 18.02 | 30.85 | 12.83 | −8.17 | 2.9E + 02 |
| 36 | Colo 320DM | colon | 17.01 | 36.06 | 19.05 | −1.95 | 3.9E + 00 |
| 37 | Colo201 | colon | 17.89 | 30.55 | 12.66 | −8.34 | 3.2E + 02 |
| 38 | Cx-1 | colon | 18.79 | 33.59 | 14.8 | −6.2 | 7.4E + 01 |
| 39 | ddH2O | colon | 40 | 40 | 0 | −21 | ND |
| 40 | HCT116 | colon | 17.59 | 30.34 | 12.75 | −8.25 | 3.0E + 02 |
| 41 | HCT116/epo5 | colon | 17.71 | 30.62 | 12.91 | −8.09 | 2.7E + 02 |
| 42 | HCT116/ras | colon | 17.18 | 30.05 | 12.87 | −8.13 | 2.8E + 02 |
| 43 | HCT116/TX1 | colon | 17.36 | 30.04 | 12.68 | −8.32 | 3.2E + 02 |
| | 5CR | | | | | | |
| 44 | HCT116/vivo | colon | 17.7 | 30.31 | 12.61 | −8.39 | 3.4E + 02 |
| 45 | HCT116/VM4 | colon | 17.87 | 31.01 | 13.14 | −7.86 | 2.3E + 02 |
| | 6 | | | | | | |
| 46 | HCT116/VP3 | colon | 17.3 | 32.13 | 14.83 | −6.17 | 7.2E + 01 |
| | 5 | | | | | | |
| 47 | HCT-8 | colon | 17.44 | 29.13 | 11.69 | −9.31 | 6.3E + 02 |
| 48 | HT-29 | colon | 17.9 | 37.17 | 19.27 | −1.73 | 3.3E + 00 |
| 49 | LoVo | colon | 17.64 | 30.35 | 12.71 | −8.29 | 3.1E + 02 |
| 50 | LS 174T | colon | 17.93 | 29.2 | 11.27 | −9.73 | 8.5E + 02 |
| 51 | LS123 | colon | 17.65 | 28.34 | 10.69 | −10.31 | 1.3E + 03 |
| 52 | MIP | colon | 16.92 | 37.01 | 20.09 | −0.91 | 1.9E + 00 |
| 53 | SK-CO-1 | colon | 17.75 | 29.09 | 11.34 | −9.66 | 8.1E + 02 |
| 54 | SW1417 | colon | 17.22 | 37.66 | 20.44 | −0.56 | 1.5E + 00 |
| 55 | SW403 | colon | 18.39 | 29.61 | 11.22 | −9.78 | 8.8E + 02 |
| 56 | SW480 | colon | 17 | 29.19 | 12.19 | −8.81 | 4.5E + 02 |
| 57 | SW620 | colon | 17.16 | 30.23 | 13.07 | −7.93 | 2.4E + 02 |
| 58 | SW837 | colon | 18.35 | 28.33 | 9.98 | −11.02 | 2.1E + 03 |
| 59 | T84 | colon | 16.44 | 28.08 | 11.64 | −9.36 | 6.6E + 02 |
| 60 | CCD-18Co | colon, | 17.19 | 40 | 22.81 | 1.81 | 0.0E + 00 |
| | | fibroblast | | | | | |
| 61 | HT-1080 | fibrosarcoma | 17.16 | 34.03 | 16.87 | −4.13 | 1.8E + 01 |
| 62 | CCRF-CEM | leukemia | 17.07 | 30.98 | 13.91 | −7.09 | 1.4E + 02 |
| 63 | HL-60 | leukemia | 17.54 | 30.59 | 13.05 | −7.95 | 2.5E + 02 |
| 64 | K562 | leukemia | 18.42 | 30.28 | 11.86 | −9.14 | 5.6E + 02 |
| 65 | A-427 | lung | 18 | 40 | 22 | 1 | 0.0E + 00 |
| 66 | A549 | lung | 17.63 | 33.38 | 15.75 | −5.25 | 3.8E + 01 |
| 67 | Calu-3 | lung | 18.09 | 31.16 | 13.07 | −7.93 | 2.4E + 02 |
| 68 | Calu-6 | lung | 16.62 | 34.01 | 17.39 | −3.61 | 1.2E + 01 |
| 69 | ChaGo-K-1 | lung | 17.79 | 33.79 | 16 | −5 | 3.2E + 01 |
| 70 | DMS 114 | lung | 18.14 | 37.5 | 19.36 | −1.64 | 3.1E + 00 |
| 71 | LX-1 | lung | 18.17 | 31.61 | 13.44 | −7.56 | 1.9E + 02 |
| 72 | MRC-5 | lung | 17.3 | 38.87 | 21.57 | 0.57 | 6.7E − 01 |
| 73 | MSTO-211H | lung | 16.81 | 38.43 | 21.62 | 0.62 | 6.5E − 01 |
| 74 | NCI-H596 | lung | 17.73 | 35.66 | 17.93 | −3.07 | 8.4E + 00 |
| 75 | SHP-77 | lung | 18.66 | 37.89 | 19.23 | −1.77 | 3.4E + 00 |
| 76 | Sk-LU-1 | lung | 15.81 | 37.95 | 22.14 | 1.14 | 4.5E − 01 |
| 77 | SK-MES-1 | lung | 17.1 | 36.54 | 19.44 | −1.56 | 2.9E + 00 |
| 78 | SW1271 | lung | 16.45 | 40 | 23.55 | 2.55 | 0.0E + 00 |
| 79 | SW1573 | lung | 17.14 | 40 | 22.86 | 1.86 | 0.0E + 00 |
| 80 | SW900 | lung | 18.17 | 40 | 21.83 | 0.83 | 0.0E + 00 |
| 81 | Hs 294T | melanoma | 17.73 | 35.42 | 17.69 | −3.31 | 9.9E + 00 |
| 82 | A2780/DDP-R | ovarian | 21.51 | 40 | 18.49 | −2.51 | 0.0E + 00 |
| 83 | A2780/DDP-S | ovarian | 17.89 | 40 | 22.11 | 1.11 | 0.0E + 00 |
| 84 | A2780/epo5 | ovarian | 17.54 | 33.94 | 16.4 | −4.6 | 2.4E + 01 |
| 85 | A2780/TAX- | ovarian | 18.4 | 38.49 | 20.09 | −0.91 | 1.9E + 00 |
| | R | | | | | | |
| 86 | A2780/TAX-S | ovarian | 17.83 | 38.67 | 20.84 | −0.16 | 1.1E + 00 |
| 87 | Caov-3 | ovarian | 15.5 | 30.23 | 14.73 | −6.27 | 7.7E + 01 |
| 88 | ES-2 | ovarian | 17.22 | 38.34 | 21.12 | 0.12 | 9.2E − 01 |
| 89 | HOC-76 | ovarian | 34.3 | 40 | 5.7 | −15.3 | 0.0E + 00 |
| 90 | OVCAR-3 | ovarian | 17.09 | 37.09 | 20 | −1 | 2.0E + 00 |
| 91 | PA-1 | ovarian | 17.33 | 31.05 | 13.72 | −7.28 | 1.6E + 02 |
| 92 | SW 626 | ovarian | 16.94 | 29.91 | 12.97 | −8.03 | 2.6E + 02 |
| 93 | UPN251 | ovarian | 17.69 | 39.07 | 21.38 | 0.38 | 7.7E − 01 |
| 94 | LNCAP | prostate | 18.17 | 38.5 | 20.33 | −0.67 | 1.6E + 00 |
| 95 | PC-3 | prostate | 17.25 | 32.27 | 15.02 | −5.98 | 6.3E + 01 |
| 96 | A431 | squamous | 19.85 | 32.25 | 12.4 | −8.6 | 3.9E + 02 |

### Example 6

### Signal Transduction Assays

The activity of GPCRs or homologues thereof, can be measured using any assay suitable for the measurement of the activity of a G protein-coupled receptor, as commonly known in the art. Signal transduction activity of a G protein-coupled receptor can be monitor by monitoring intracellular Ca2+, cAMP, inositol 1,4,5-triphosphate (IP₃), or 1,2-diacylglycerol (DAG). Assays for the measurement of intracellular Ca²⁺ are described in Sakurai et al. (EP 480 381). Intracellular IP₃ can be measured using a kit available from Amersham, Inc. (Arlington Heights, Ill.). A kit for measuring intracellular cAMP is available from Diagnostic Products, Inc. (Los Angeles, Calif.)

Activation of a G protein-coupled receptor triggers the release of Ca²⁺ ions sequestered in the mitochondria, endoplasmic reticulum, and other cytoplasmic vesicles into the cytoplasm. Fluorescent dyes, e.g., fura-2, can be used to measure the concentration of free cytoplasmic Ca²⁺. The ester of fura-2, which is lipophilic and can diffuse across the cell membrane, is added to the media of the host cells expressing GPCRs. Once inside the cell, the fura-2 ester is hydrolyzed by cytosolic esterases to its non-lipophilic form, and then the dye cannot diffuse back out of the cell. The non-lipophilic form of fura-2 will fluoresce when it binds to free Ca²⁺. The fluorescence can be measured without lysing the cells at an excitation spectrum of 340 nm or 380 nm and at fluorescence spectrum of 500 nm (Sakurai et al., EP 480 381).

Upon activation of a G protein-coupled receptor, the rise of free cytosolic Ca²⁺ concentrations is preceded by the hydrolysis of phosphatidylinositol 4,5-bisphosphate. Hydrolysis of this phospholipid by the phospholipase C yields 1,2-diacylglycerol (DAG), which remains in the membrane, and water-soluble inositol 1,4,5-triphosphate (IP₃). Binding of ligands or agonists will increase the concentration of DAG and IP₃. Thus, signal transduction activity can be measured by monitoring the concentration of these hydrolysis products.

To measure the IP₃ concentrations, radioactivity labeled ³H-inositol is added to the media of host cells expressing GPCRs. The ³H-inositol is taken up by the cells and incorporated into IP₃. The resulting inositol triphosphate is separated from the mono and di-phosphate forms and measured (Sakurai et al., EP 480 381). Alternatively, Amersham provides an inositol 1,4,5-triphosphate assay system. With this system Amersham provides tritylated inositol 1,4,5-triphosphate and a receptor capable of distinguishing the radioactive inositol from other inositol phosphates. With these reagents an effective and accurate competition assay can be performed to determine the inositol triphosphate levels.

Cyclic AMP levels can be measured according to the methods described in Gilman et al., Proc. Natl. Acad. Sci 67:305-312 (1970). In addition, a kit for assaying levels of cAMP is available from Diagnostic Products Corp. (Los Angeles, Calif.).

### Example 7

### GPCR Activity

Another method for screening compounds which are antagonists, and thus inhibit activation of the receptor polypeptide of the present invention is provided. This involves determining inhibition of binding of labeled ligand, such as dATP, dAMP, or UTP, to cells which have the receptor on the surface thereof, or cell membranes containing the receptor. Such a method further involves transfecting a eukaryotic cell with DNA encoding the GPCR polypeptide such that the cell expresses the receptor on its surface. The cell is then contacted with a potential antagonist in the presence of a labeled form of a ligand, such as dATP, dAMP, or UTP. The ligand can be labeled, e.g., by radioactivity, fluorescence, or any detectable label commonly known in the art. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand which binds to the receptors. This method is called a binding assay. Naturally, this same technique can be used to determine agonists.

In a further screening procedure, mammalian cells, for example, but not limited to, CHO, HEK 293, Xenopus Oocytes, RBL-2H3, etc., which are transfected, are used to express the receptor of interest. The cells are loaded with an indicator dye that produces a fluorescent signal when bound to calcium, and the cells are contacted with a test substance and a receptor agonist, such as DATP, DAMP, or UTP. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

In yet another screening procedure, mammalian cells are transfected to express the receptor of interest, and are also transfected with a reporter gene construct that is coupled to activation of the receptor (for example, but not limited to luciferase or beta-galactosidase behind an appropriate promoter). The cells are contacted with a test substance and the receptor agonist (ligand), such as dATP, dAMP, or UTP, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another screening technique for antagonists or agonists involves introducing RNA encoding the GPCR polypeptide into cells (or CHO, HEK 293, RBL-2H3, etc.) to transiently or stably express the receptor. The receptor cells are then contacted with the receptor ligand, such as dATP, dAMP, or UTP, and a compound to be screened. Inhibition or activation of the receptor is then determined by detection of a signal, such as, cAMP, calcium, proton, or other ions.

### Example 8

### Functional Characterization of HGPRBMY3

DNA Constructs:

The putative GPCR HGPRBMY3 cDNA was PCR amplified using PFU™ (Stratagene). The primers used in the PCR reaction were specific to the HGPRBMY3 polynucleotide and were ordered from Gibco BRL (5′-gtccccaagcttgcaccatgttagccaacagctcctcaaccaacagttc-3′ (SEQ ID NO:33). The following 3 prime primer was used to add a Flag-tag epitope to the HGPRBMY3 polypeptide for immunocytochemistry: 5′-gtccgcggatccctacttgtcgtcgtcgtccttgtagtccatgagggcggaatcctggggacactgtgtgaa-3′(SEQ ID NO:34). The product from the PCR reaction was isolated from a 0.8% Agarose gel (Invitrogen) and purified using a Gel Extraction Kit™ from Qiagen.

The purified product was then digested overnight along with the pcDNA3.1 Hygro™ mammalian expression vector from Invitrogen using the HindIII and BamHI restriction enzymes (New England Biolabs). These digested products were then purified using the Gel Extraction Kit™ from Qiagen and subsequently ligated to the pcDNA3.1 Hygro™ expression vector using a DNA molar ratio of 4 parts insert: 1 vector. All DNA modification enzymes were purchased from NEB. The ligation was incubated overnight at 16 degrees Celsius, after which time, one microliter of the mix was used to transform DH5 alpha cloning efficiency competent E. coli™ (Gibco BRL). A detailed description of the pcDNA3.1 Hygro™ mammalian expression vector is available at the Invitrogen web site (www.Invitrogen.com). The plasmid DNA from the ampicillin resistant clones were isolated using the Wizard DNA Miniprep System™ from Promega. Positive clones were then confirmed and scaled up for purification using the Qiagen Maxiprep TM plasmid DNA purification kit.

Cell Line Generation:

The pcDNA3.1 hygro vector containing the orphan HGPRBMY3 cDNA was used to transfect CHO-NFAT/CRE or the CHO-NFAT G alpha 15 (Aurora Biosciences) cells using Lipofectamine 2000™ according to the manufacturers specifications (Gibco BRL). Two days later, the cells were split 1:3 into selective media (DMEM 11056, 600 μg/ml Hygromycin, 200 μg/ml Zeocin, 10% fetal bovine serum, FBS). All cell culture reagents were purchased from Gibco BRL-Invitrogen.

The CHO-NFAT/CRE or CHO-NFAT G alpha 15 cell lines, transiently or stably transfected with the orphan HGPRBMY3 GPCR, were analyzed using the FACS Vantage SE™ (BD), fluorescence microscopy (Nikon), and the LJL Analyst™ (Molecular Devices). In this system, changes in real-time gene expression, as a consequence of constitutive G-protein coupling of the orphan HGPRBMY3 GPCR, is examined by analyzing the fluorescence emission of the transformed cells at 447 nm and 518 nm. The changes in gene expression can be visualized using Beta-Lactamase as a reporter, that, when induced by the appropriate signaling cascade, hydrolyzes an intracellularly loaded, membrane-permeant ester substrate, Cephalosporin-Coumarin-Fluorescein2/Acetoxymethyl (CCF2/AM™ Aurora Biosciences; 7). The CCF2/AMTM substrate is a 7-hydroxycoumarin cephalosporin with a fluorescein attached through a stable thioether linkage. Induced expression of the Beta-Lactamase enzyme is readily apparent since each enzyme molecule produced is capable of changing the fluorescence of many CCF2/AM™ substrate molecules. A schematic of this cell based system is shown below.

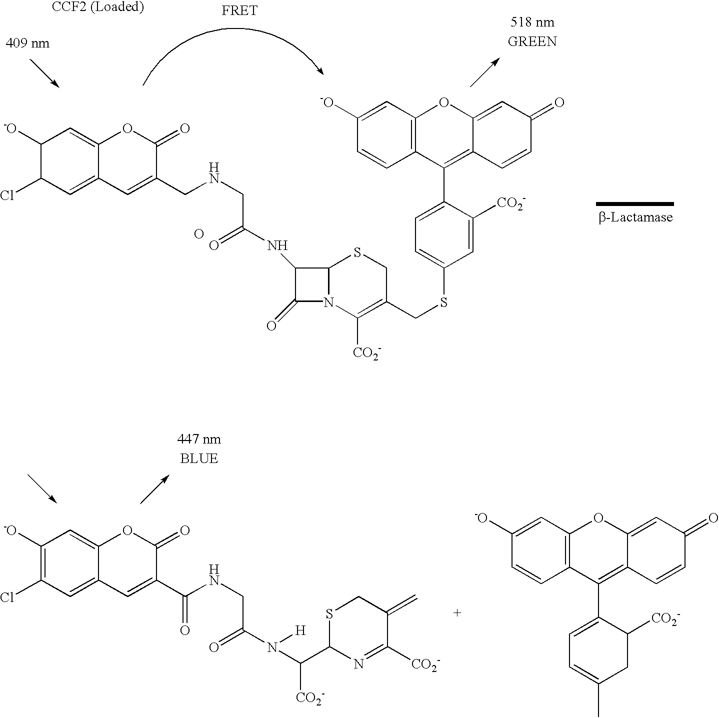

In summary, CCF2/AM™ is a membrane permeant, intracellularly-trapped, fluorescent substrate with a cephalosporin core that links a 7-hydroxycoumarin to a fluorescein. For the intact molecule, excitation of the coumarin at 409 nm results in Fluorescence Resonance Energy Transfer (FRET) to the fluorescein which emits green light at 518 nm. Production of active Beta-Lactamase results in cleavage of the Beta-Lactam ring, leading to the disruption of FRET, and excitation of the coumarin only—thus giving rise to blue fluorescent emission at 447 nm.

Fluorescent emissions were detected using a Nikon-TE300 microscope equipped with an excitation filter (D405/10X-25), dichroic reflector (430DCLP), and a barrier filter for dual DAPI/FITC (510 nM) to visually capture changes in Beta-Lactamase expression. The FACS Vantage SE is equipped with a Coherent Enterprise II Argon Laser and a Coherent 302C Krypton laser. In flow cytometry, LV excitation at 351-364 nm from the Argon Laser or violet excitation at 407 nm from the Krypton laser are used. The optical filters on the FACS Vantage SE are HQ460/50m and HQ535/40m bandpass separated by a 490 dichroic mirror.

Prior to analyzing the fluorescent emissions from the cell lines as described above, the cells were loaded with the CCF2/AM substrate. A 6× CCF2/AM loading buffer was prepared whereby 1 mM CCF2/AM (Aurora Biosciences) was dissolved in 100% DMSO (Sigma). Stock solution (12 μl) was added to 60 μl of 100 mg/ml Pluronic F127 (Sigma) in DMSO containing 0.1% Acetic Acid (Sigma). This solution was added while vortexing to 1 mL of Sort Buffer (PBS minus calcium and magnesium-Gibco-25 mM HEPES-Gibco-pH 7.4, 0.1% BSA). Cells were placed in serum-free media and the 6× CCF2/AM was added to a final concentration of IX. The cells were then loaded at room temperature for one to two hours, and then subjected to fluorescent emission analysis as described herein. Additional details relative to the cell loading methods and/or instrument settings may be found by reference to the following publications: see Zlokarnik, et al., 1998; Whitney et al., 1998; and BD Biosciences,1999.

Immunocytochemistry:

The cell lines transfected and selected for expression of Flag-epitope tagged orphan GPCRs were analyzed by immunocytochemistry. The cells were plated at 1×10³ in each well of a glass slide (VWR). The cells were rinsed with PBS followed by acid fixation for 30 minutes at room temperature using a mixture of 5% Glacial Acetic Acid/90% ethanol. The cells were then blocked in 2% BSA and 0.1% Triton in PBS, incubated for 2 h at room temperature or overnight at 4° C. A monoclonal anti-Flag FITC antibody was diluted at 1:50 in blocking solution and incubated with the cells for 2 h at room temperature. Cells were then washed three times with 0.1% Triton in PBS for five minutes. The slides were overlaid with mounting media dropwise with Biomedia-Gel Mount™ (Biomedia; Containing Anti-Quenching Agent). Cells were examined at 10× magnification using the Nikon TE300 equipped with FITC filter (535 nm).

There is strong evidence that certain GPCRs exhibit cDNA concentration-dependent constitutive activity through cAMP response element (CRE) luciferase reporters (Chen et al., 1999). In an effort to demonstrate functional coupling of HGPRBMY3 to known GPCR second messenger pathways, the HGPRBMY3 polypeptide was expressed at high constitutive levels in the CHO-NFAT/CRE cell line. To this end, the HGPRBMY3 cDNA was PCR amplified and subcloned into the pcDNA3.1 hygro™ mammalian expression vector as described herein. Early passage CHO-NFAT/CRE cells were then transfected with the resulting pcDNA3.1 hygro™/HGPRBMY3 construct. Transfected and non-transfected CHO-NFAT/CRE cells (control) were loaded with the CCF2 substrate and stimulated with 10 μM PMA, 1 μM Thapsigargin (NFAT stimulator), and 10 μM Forskolin (CRE stimulator) to fully activate the NFAT/CRE element. The cells were then analyzed for fluorescent emission by FACS.

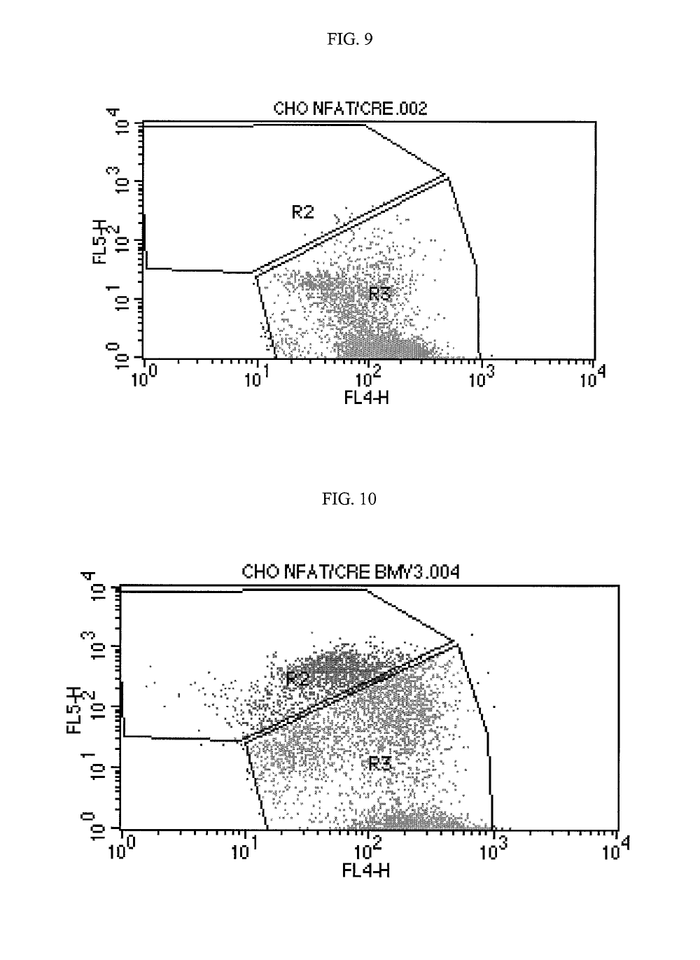

The FACS profile demonstrates the constitutive activity of HGPRBMY3 in the CHO-NFAT/CRE line as evidenced by the significant population of cells with blue fluorescent emission at 447 nm (see FIG. 10: Blue Cells). FIG. 10 describes CHO-NFAT/CRE cell lines transfected with the pcDNA3.1 Hygro™/HGPRBMY3 mammalian expression vector, as described herein. The cells were analyzed via FACS (Fluorescent Assisted Cell Sorter) according to their wavelength emission at 518 nM (Channel R3-Green Cells), and 447 nM (Channel R2-Blue Cells). As shown, overexpression of HGPRBMY3 results in functional coupling and subsequent activation of beta lactamase gene expression, as evidenced by the significant number of cells with fluorescent emission at 447 nM relative to the non-transfected control CHO-NFAT/CRE cells (shown in FIG. 9). FIG. 9 describes control CHO-NFAT/CRE (Nuclear Factor Activator of Transcription (NFAT)/cAMP response element (CRE)) cell lines, in the absence of the pcDNA3.1 Hygro™/HGPRBMY3 mammalian expression vector transfection. As shown, the vast majority of cells emit at 518 nM, with minimal emission observed at 447 nM. The latter is expected since the NFAT/CRE Response Elements remain dormant in the absence of an activated G-protein dependent signal transduction pathway (e.g., pathways mediated by Gq/11 or Gs coupled receptors). As a result, the cell permeant, CCF2/AM™ (Aurora Biosciences; 7) substrate remains intact and emits light at 518 nM.

As expected, the NFAT/CRE response element in the untransfected control cell line was not activated (i.e., beta lactamase not induced), enabling the CCF2 substrate to remain intact, and resulting in the green fluorescent emission at 518 nM (see FIG. 9—Green Cells). A very low level of leaky Beta Lactamase expression was detectable as evidenced by the small population of cells emitting at 447 nm. Analysis of a stable pool of cells transfected with HGPRBMY3 revealed constitutive coupling of the cell population to the NFAT/CRE response element, activation of Beta Lactamase and cleavage of the substrate (FIG. 10—Blue Cells). These results demonstrate that overexpression of HGPRBMY3 leads to constitutive coupling of signaling pathways known to be mediated by Gq/11 or Gs coupled receptors that converge to activate either the NFAT or CRE response elements respectively (Boss et al., 1996; Chen et al., 1999).

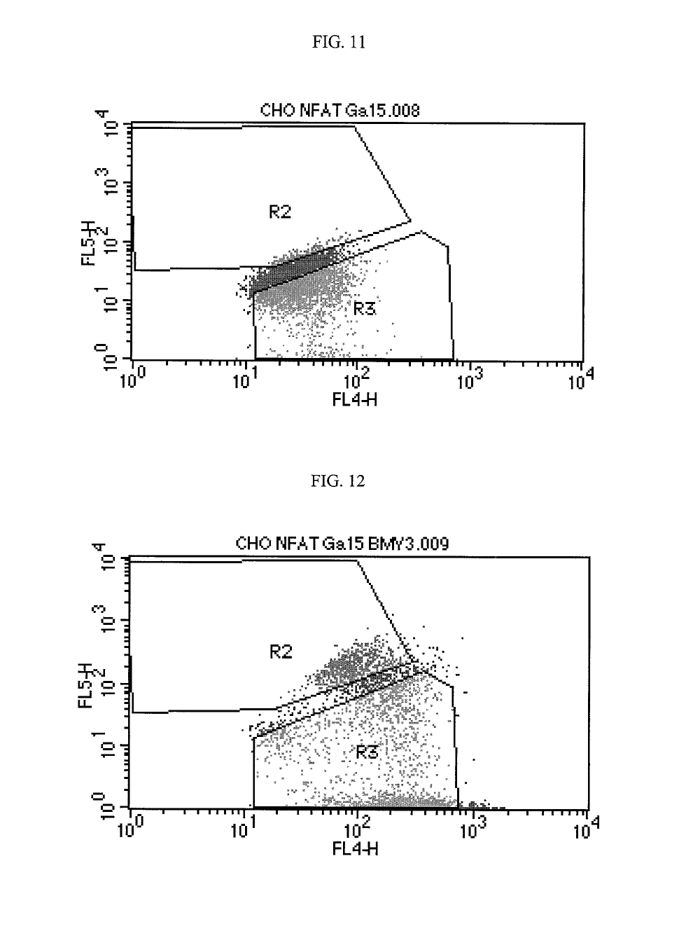

In an effort to further characterize the observed functional coupling of the HGPRBMY3 polypeptide, its ability to couple to a G protein was examined. To this end, the promiscuous G protein, G alpha 15 was utilized. Specific domains of alpha subunits of G proteins have been shown to control coupling to GPCRs (Blahos et al., 2001). It has been shown that the extreme C-terminal 20 amino acids of either G alpha 15 or 16 confer the unique ability of these G proteins to couple to many GPCRs, including those that naturally do not stimulate phospholipase C, PLC (Blahos et al., 2001). Indeed, both G alpha 15 and 16 have been shown to couple a wide variety of GPCRs to Phospholipase C activation of calcium mediated signaling pathways (including the NFAT-signaling pathway) (Offermanns & Simon). To demonstrate that HGPRBMY3 was functioning as a GPCR, the CHO-NFAT G alpha 15 cell line that contained only the integrated NFAT response element linked to the Beta-Lactamase reporter was transfected with the pcDNA3.1 hygro TM/HGPRBMY3 construct. Analysis of the fluorescence emission from this stable pool showed that HGPRBMY3 constitutively coupled to the NFAT mediated second messenger pathways via G alpha 15 (see FIGS. 11 and 12). FIG. 11 describes control CHO-NFAT G alpha 15 (Nuclear Factor Activator of Transcription (NFAT)) cell lines, in the absence of the pcDNA3.1 Hygro™/HGPRBMY3 mammalian expression vector transfection. The cells were analyzed via FACS (Fluorescent Assisted Cell Sorter) according to their wavelength emission at 518 nM (Channel R3-Green Cells), and 447 nM (Channel R2-Blue Cells). As shown, the vast majority of cells emit at 518 nM, with minimal emission observed at 447 nM. The latter is expected since the NFAT response elements remain dormant in the absence of an activated G-protein dependent signal transduction pathway (e.g., pathways mediated by G alpha 15 Gq/11 or Gs coupled receptors). As a TM result, the cell permeant, CCF2/AM™ (Aurora Biosciences; 7) substrate remains intact and emits light at 518 nM. FIG. 12 describes CHO-NFAT G alpha 15 cell lines transfected with the pcDNA3.1 Hygro™/HGPRBMY3 mammalian expression vector. The cells were analyzed and sorted via FACS according to their wavelength emission at 518 nM (Channel R3-Green Cells), and 447 nM (Channel R2-Blue Cells). As shown, overexpression of HGPRBMY3 results in functional coupling and subsequent activation of beta lactamase gene expression, as evidenced by the significant number of cells with fluorescent emission at 447 nM relative to the non-transfected control CHO-NFAT G alpha 15 cells (shown in FIG. 11).

In conclusion, the results are consistent with HGPRBMY3 representing a functional GPCR analogous to known G alpha 15 coupled receptors. Therefore, constitutive expression of HGPRBMY3 in the CHO-NFAT G alpha 15 cell line leads to NFAT activation through accumulation of intracellular Ca²⁺ as has been demonstrated for the M3 muscarinic receptor (4).

Demonstration of Cellular Expression:

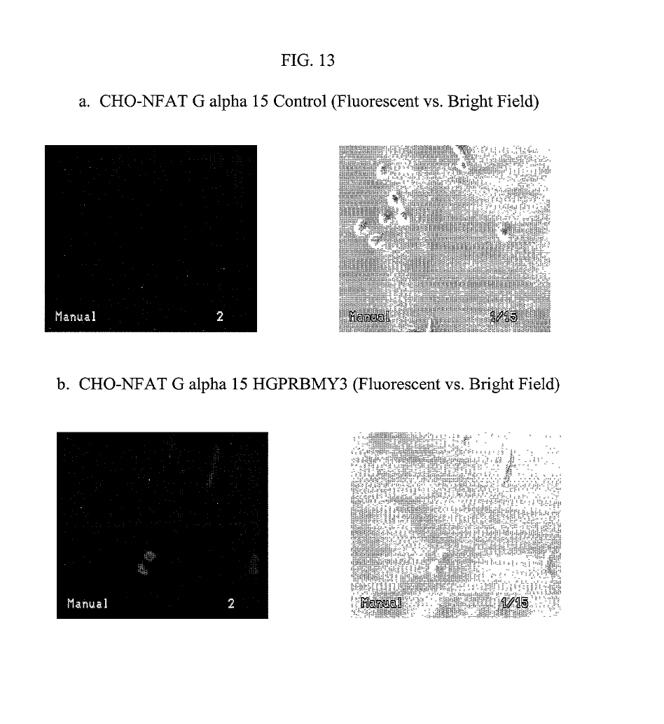

HGPRBMY3 was tagged at the C-terminus using the Flag epitope and inserted into the pcDNA3.1 hygro™ expression vector, as described herein. Immunocytochemistry of CHO-NFAT G alpha 15 cell lines transfected with the Flag-tagged HGPRBMY3 construct with FITC conjugated monoclonal antibody directed agains FLAG demonstrated that HGPRBMY3 is indeed a cell surface receptor. The immunocytochemistry also confirmed expression of the HGPRBMY3 in the CHO-NFAT G alpha 15 cell lines. Briefly, CHO-NFAT G alpha 15 cell lines were transfected with pcDNA3.1 hygro™/HGPRBMY3-Flag vector, fixed with 70% methanol, and permeablized with 0.1% TritonX100. The cells were then blocked with 1% Serum and incubated with a FITC conjugated monoclonal antibody raised against FLAG at 1:50 dilution in PBS-Triton. The cells were then washed several times with PBS-Triton, overlayed with mounting solution, and fluorescent images were captured (see FIG. 13). The control cell line, non-transfected CHO-NFAT G alpha 15 cell line, exhibited no detectable background fluorescence (FIG. 13). The HGPRBMY3-FLAG tagged expressing CHO-NFAT G alpha 15 line exhibited specific plasma membrane expression as indicated (FIG. 13). Panel A shows the transfected CHO-NFAT/CRE cells under visual wavelengths, and panel B shows the fluorescent emission of the same cells at 530 μm after illumination with a mercury light source. The cellular localization is clearly evident in panel B, and is consistent with the HGPRBMY3 polypeptide representing a member of the GPCR family. These data provide clear evidence that HGPRBMY3 is expressed in these cells and the majority of the protein is localized to the cell surface. Cell surface localization in consistent with HGPRBMY3 representing a 7 transmembrane domain containing GPCR. Taken together, the data indicate that HGPRBMY3 is a cell surface GPCR that can function through increases in Ca²⁺ signal transduction pathways via G alpha 15.

Screening Paradigm

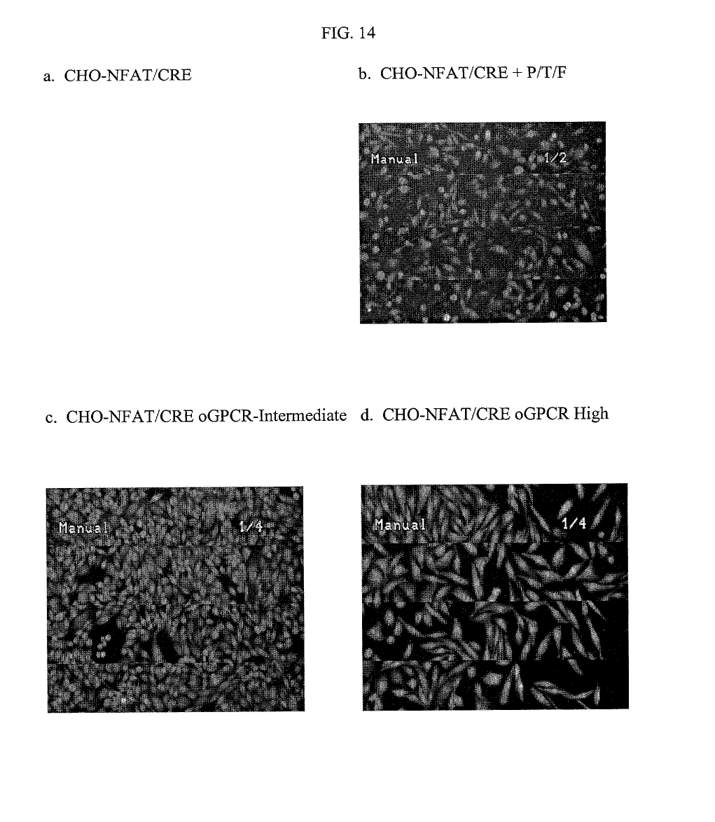

The Aurora Beta-Lactamase technology provides a clear path for identifying agonists and antagonists of the HGPRBMY3 polypeptide. Cell lines that exhibit a range of constitutive coupling activity have been identified by sorting through HGPRBMY3 transfected cell lines using the FACS Vantage SE (see FIG. 14). FIG. 14 describes several CHO-NFAT/CRE cell lines transfected with the pcDNA3.1 Hygro™/HGPRBMY3 mammalian expression vector were isolated via FACS that had either intermediate or high beta lactamase expression levels of constitutive activation, as described herein. Panel A shows untransfected CHO-NFAT/CRE cells prior to stimulation with 10 nM PMA, 1 μM Thapsigargin, and 10 μM Forskolin (−P/T/F). Panel B shows CHO-NFAT/CRE cells after stimulation with 10 nM PMA, 1 μM Thapsigargin, and 10 μM Forskolin (+P/T/F). Panel C shows a representative orphan GPCR (oGPCR) transfected CHO-NFAT/CRE cells that have an intermediate level of beta lactamase expression. Panel D shows a representative oGPCR transfected CHO-NFAT/CRE that have a high level of beta lactamase expression. For example, cell lines have been sorted that have an intermediate level of orphan GPCR expression, which also correlates with an intermediate coupling response, using the LJL analyst. Such cell lines will provide the opportunity to screen, indirectly, for both agonists and antagonists of HGPRBMY3 by looking for inhibitors that block the beta lactamase response, or agonists that increase the beta lactamase response. As described herein, modulating the expression level of beta lactamase directly correlates with the level of cleaved CCF2 substrate. For example, this screening paradigm has been shown to work for the identification of modulators of a known GPCR, 5HT6, that couples through Adenylate Cyclase, in addition to, the identification of modulators of the 5HT2c GPCR, that couples through changes in [Ca 2+]i. The data shown below represent cell lines that have been engineered with the desired pattern of HGPRBMY3 expression to enable the identification of potent small molecule agonists and antagonists. HGPRBMY3 modulator screens may be carried out using a variety of high throughput methods known in the art, though preferably using the fully automated Aurora UHTSS system. The uninduced, orphan-transfected CHO-NFAT/CRE cell line represents the relative background level of beta lactamase expression (FIG. 14; panel a). Following treatment with a cocktail of 10 nM PMA, 1 μM Thapsigargin, and 10 μM Forskolin (FIG. 14; P/T/F; panel b), the cells fully activate the CRE-NFAT response element demonstrating the dynamic range of the assay. Panel C (FIG. 14) represents an orphan transfected CHO-NFAT/CRE cell line that shows an intermediate level of beta lactamase expression post P/T/F stimulation, while panel D (FIG. 14) represents a HGPRBMY3 transfected CHO-NFAT/CRE cell line that shows a high level of beta lactamase expression post P/T/F stimulation.

### Example 9

### Phage Display Methods for Identifying Peptide Ligands or Modulators of Orphan GPCRs

Library Construction

Two HGPRBMY libraries were used for identifying peptides that may function as modulators. Specifically, a 15-mer library was used to identify peptides that may function as agonists or antagonists. The 15-mer library is an aliquot of the 15-mer library originally constructed by G. P. Smith (Scott, J K and Smith, G P. 1990, Science 249:386-390). A 40-mer library was used for identifying natural ligands and constructed essentially as previously described (BK Kay, et al. 1993, Gene 128:59-65), with the exception that a 15 base pair complementary region was used to anneal the two oligonucleotides, as opposed to 6, 9, or 12 base pairs, as described below.

The oligos used were: Oligo 1: 5′-CGAAGCGTAAGGGCCCAGCCGGCC (NNK x 20) CCGGGTCCGGGCGGC-3′ (SEQ ID NO:35) and Oligo2: 5′-AAAAGGAAAAAAGCGGCCGC (VNN x 20) GCCGCCCGGACCCGG-3′ (SEQ ID NO:36), where N=A+G+C+T and K=C+G+T and V=C+A+G.

The oligos were annealed through their 15 base pair complimentary sequences which encode a constant ProGlyProGlyGly (SEQ ID NO:37) pentapeptide sequence between the random 20 amino acid segments, and then extended by standard procedure using Klenow enzyme. This was followed by endonuclease digestion using Sfi1 and Not1 enzymes and ligation to Sfi1 and Not1 cleaved pCantab5E (Pharmacia). The ligation mixture was electroporated into E. coli XL1Blue and phage clones were essentially generated as suggested by the manufacturer for making ScFv antibody libraries in pCantab5E.

Sequencing Bound Phage

Standard procedures commonly known in the art were used. Phage in eluates were infected into E. coli host strain (TG1 for the 15-mer library; XL1Blue for the 40-mer library) and plated for single colonies. Colonies were grown in liquid and sequenced by standard procedure which involved: 1) generating PCR product with suitable primers of the library segments in the phage genome (15 mer library) or pCantab5E (40 mer library); and 2) sequencing PCR products using one primer of each PCR primer pair. Sequences were visually inspected or by using the Vector NTI alignment tool.

Peptide Modulators

The following serve as non-limiting examples of peptides:

| | |
|---|---|
| GDFWYEACESSCAFW | (SEQ ID NO:38) |
| LEWGSDVFYDVYDCC | (SEQ ID NO:39) |
| CLRSGTGCAFQLYRF | (SEQ ID NO:40) |
| LFSSETFFDACCAFE | (SEQ ID NO:41) |
| RIDCCAKYFLRSCD | (SEQ ID NO:42) |

Peptide Synthesis

Peptides were synthesized on Fmoc-Knorr amide resin [N-(9-fluorenyl)methoxycarbonyl-Knorr amide-resin; Midwest Biotech; Fishers, IN] with an Applied Biosystems (Foster City, Calif.) model 433A synthesizer and the FastMoc chemistry protocol (0.25 mmol scale) supplied with the instrument. Amino acids were double coupled as their N-α-Fmoc-derivatives and reactive side chains were protected as follows: Asp, Glu: t-Butyl ester (OtBu); Ser, Thr, Tyr: t-Butyl ether (tBu); Asn, Cys, Gln, His: Triphenylmethyl (Trt); Lys, Trp: t-Butyloxycarbonyl (Boc); Arg: 2,2,4,6,7-Pentamethyldihydrobenzofuran-5-sulfonyl (Pbf). After the final double coupling cycle, the N-terminal Fmoc group was removed by the multi-step treatment with piperidine in N-Methylpyrrolidone described by the manufacturer. The N-terminal free amines were then treated with 10% acetic anhydride, 5% Diisopropylamine in N-Methylpyrrolidone to yield the N-acetyl-derivative. The protected peptidyl-resins were simultaneously deprotected and removed from the resin by standard methods. The lyophilized peptides were purified on C₁₈ to apparent homogeneity as judged by RP-HPLC analysis. Predicted peptide molecular weights were verified by electrospray mass spectrometry (J. Biol. Chem. 273:12041-12046, 1998)

Cyclic analogs were prepared from the crude linear products. The cysteine disulfide was formed using one of the following methods:

Method 1:

A sample of the crude peptide was dissolved in water at a concentration of 0.5 mg/mL and the pH adjusted to 8.5 with NH₄OH. The reaction was stirred at room temperature, and monitored by RP-HPLC. Once completed, the reaction was adjusted to pH 4 with acetic acid and lyophilized. The product was purified and characterized as above.

Method 2:

A sample of the crude peptide was dissolved at a concentration of 0.5 mg/mL in 5% acetic acid. The pH was adjusted to 6.0 with NH₄OH. DMSO (20% by volume) was added and the reaction was stirred overnight. After analytical RP-HPLC analysis, the reaction was diluted with water and triple lyophilized to remove DMSO. The crude product was purified by preparative RP-HPLC (JACS. 113:6657, 1991)

Assessing Affect of Peptides on GPCR Function.

The effect of any one of these peptides on the function of the GPCR of the present invention may be determined by adding an effective amount of each peptide to each functional assay. Representative functional assays are described more specifically herein, particularly Example 8.

Uses Of The Peptide Modulators Of The Present Invention.

The aforementioned peptides of the present invention are useful for a variety of purposes, though most notably for modulating the function of the GPCR of the present invention, and potentially with other GPCRs of the same G-protein coupled receptor subclass (e.g., peptide receptors, adrenergic receptors, purinergic receptors, etc.), and/or other subclasses known in the art. For example, the peptide modulators of the present invention may be useful as HGPRBMY3 agonists. Alternatively, the peptide modulators of the present invention may be useful as HGPRBMY3 antagonists of the present invention. In addition, the peptide modulators of the present invention may be useful as competitive inhibitors of the HGPRBMY3 cognate ligand(s), or may be useful as non-competitive inhibitors of the HGPRBMY3 cognate ligand(s).

Furthermore, the peptide modulators of the present invention may be useful in assays designed to either deorphan the HGPRBMY3 polypeptide of the present invention, or to identify other agonists or antagonists of the HGPRBMY3 polypeptide of the present invention, particularly small molecule modulators.

### Example 10

### Method of Creating N- and C-Terminal Deletion Mutants Corresponding to the HGPRBMY3 Polypeptide

As described elsewhere herein, the present invention encompasses the creation of N- and C-terminal deletion mutants, in addition to any combination of N- and C-terminal deletions thereof, corresponding to the HGPRBMY3 polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutants of the present invention, exemplary methods are described below.

Briefly, using the isolated cDNA clone encoding the full-length HGPRBMY3 polypeptide sequence, appropriate primers of about 15-25 nucleotides derived from the desired 5′ and 3′ positions of SEQ ID NO: 1 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

For example, in the case of the R23 to L372 N-terminal deletion mutant, the following primers could be used to amplify a cDNA fragment corresponding to this deletion mutant:

| | |
|---|---|
| 5′ Primer | |
| 5′-GCAGCA GCGGCCGC CGCCTGCACTTGGTGGT | (SEQ ID NO:43) |

CTACAGC-3′

| | |
|---|---|
| NotI | |
| 3′ Primer | |
| 5′-GCAGCA GTCGAC GAGGGCGGAATCCTGGG | (SEQ ID NO:44) |

GACACTG-3′

SalI

For example, in the case of the M1 to A301 C-terminal deletion mutant, the following primers could be used to amplify a cDNA fragment corresponding to this deletion mutant:

| | |
|---|---|
| 5′ Primer | |
| 5′-GCAGCA GCGGCCGC ATGTTAGCCAACAGCTC | (SEQ ID NO:45) |

CTCAACC-3′

| | |
|---|---|
| NotI | |
| 3′ Primer | |
| 5′-GCAGCA GTCGAC GGCGCTAAAGTAGTACACC | (SEQ ID NO:46) |

AGCGGG-3′

SalI

Representative PCR amplification conditions are provided below, although the skilled artisan would appreciate that other conditions may be required for efficient amplification. A 100 ul PCR reaction mixture may be prepared using 10 ng of the template DNA (cDNA clone of HGPRBMY3), 200 uM 4dNTPs, 1 uM primers, 0.25U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E. coli cells using methods provided herein and/or otherwise known in the art.

The 5′ primer sequence for amplifying any additional N-terminal deletion mutants may be determined by reference to the following formula:

(S+(X*3)) to ((S+(X*3))+25),

wherein ‘S’ is equal to the nucleotide position of the initiating start codon of the HGPRBMY3 gene (SEQ ID NO:1), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO: 1. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula:

(S+(X*3)) to ((S+(X*3))−25),

wherein ‘S’ is equal to the nucleotide position of the initiating start codon of the HGPRBMY3 gene (SEQ ID NO:1), and ‘X’ is equal to the most C-terminal amino acid of the intended N-terminal deletion mutant. The first term provides the start 5′ nucleotide position of the 3′ primer, while the second term provides the end 3′ nucleotide position of the 3′ primer corresponding to the anti-sense strand of SEQ ID NO: 1. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

In preferred embodiments, the following N-terminal HGPRBMY3 deletion polypeptides are encompassed by the present invention (of SEQ ID NO:2): M1-L372, L2-L372, A3-L372, N4-L372, S5-L372, S6-L372, S7-L372, T8-L372, N9-L372, S10-L372, S11-L372, V12-L372, L13-L372, P14-L372, C15-L372, P16-L372, D17-L372, Y18-L372, R19-L372, P20-L372, T21-L372, H22-L372, R23-L372, L24-L372, H25-L372, L26-L372, V27-L372, V28-L372, Y29-L372, S30-L372, L31-L372, V32-L372, L33-L372, A34-L372, A35-L372, G36-L372, L37-L372, P38-L372, L39-L372, N40-L372, A41-L372, L42-L372, A43-L372, L44-L372, W45-L372, V46-L372, F47-L372, L48-L372, R49-L372, A50-L372, L51-L372, R52-L372, V53-L372, H54-L372, S55-L372, V56-L372, V57-L372, S58-L372, V59-L372, Y60-L372, M61-L372, C62-L372, N63-L372, L64-L372, A65-L372, A66-L372, S67-L372, D68-L372, L69-L372, L70-L372, F71-L372, T72-L372, L73-L372, S74-L372, L75-L372, P76-L372, V77-L372, R78-L372, L79-L372, S80-L372, Y81-L372, Y82-L372, A83-L372, L84-L372, H85-L372, H86-L372, W87-L372, P88-L372, F89-L372, P90-L372, D91-L372, L92-L372, L93-L372, C94-L372, Q95-L372, T96-L372, T97-L372, G98-L372, A99-L372, 1100-L372, F10-L372, Q102-L372, M103-L372, N104-L372, M105-L372, Y106-L372, G107-L372, S108-L372, C109-L372, I110-L372, F111-L372, L112-L372, M113-L372, L114-L372, 1115-L372, N116-L372, V117-L372, D118-L372, R119-L372, Y120-L372, A121-L372, A122-L372, 1123-L372, V124-L372, H125-L372, P126-L372, L127-L372, R128-L372, L129-L372, R130-L372, H131-L372, L132-L372, R133-L372, R134-L372, P135-L372, R136-L372, V137-L372, A138-L372, R139-L372, L140-L372, L141-L372, C142-L372, L143-L372, G144-L372, V145-L372, W146-L372, A147-L372, L148-L372, 1149-L372, L150-L372, V151-L372, F152-L372, A153-L372, V154-L372, P155-L372, A156-L372, A157-L372, R158-L372, V159-L372, H160-L372, R161-L372, P162-L372, S163-L372, R164-L372, C165-L372, R166-L372, Y167-L372, R168-L372, D169-L372, L170-L372, E171-L372, V172-L372, R173-L372, L174-L372, C175-L372, F176-L372, E177-L372, S178-L372, F179-L372, S180-L372, D181-L372, E182-L372, L183-L372, W184-L372, K185-L372, G186-L372, R187-L372, L188-L372, L189-L372, P190-L372, L191-L372, V192-L372, L193-L372, L194-L372, A195-L372, E196-L372, A197-L372, L198-L372, G199-L372, F200-L372, L201-L372, L202-L372, P203-L372, L204-L372, A205-L372, A206-L372, V207-L372, V208-L372, Y209-L372, S210-L372, S211-L372, G212-L372, R213-L372, V214-L372, F215-L372, W216-L372, T217-L372, L218-L372, A219-L372, R220-L372, P221-L372, D222-L372, A223-L372, T224-L372, Q225-L372, S226-L372, Q227-L372, R228-L372, R229-L372, R230-L372, K231-L372, T232-L372, V233-L372, R234L372, L235-L372, L236-L372, L237-L372, A238-L372, N239-L372, L240-L372, V241-L372, 1242-L372, F243-L372, L244-L372, L245-L372, C246-L372, F247-L372, V248-L372, P249-L372, Y250-L372, N251-L372, S252-L372, T253-L372, L254-L372, A255-L372, V256-L372, Y257-L372, G258-L372, L259-L372, L260-L372, R261-L372, S262-L372, K263-L372, L264-L372, V265-L372, A266-L372, A267-L372, S268-L372, V269-L372, P270-L372, A271-L372, R272-L372, D273-L372, R274-L372, V275-L372, R276-L372, G277-L372, V278-L372, L279-L372, M280-L372, V281-L372, M282-L372, V283-L372, L284-L372, L285-L372, A286-L372, G287-L372, A288-L372, N289-L372, C290-L372, V291-L372, L292-L372, D293-L372, P294-L372, L295-L372, V296-L372, Y297-L372, Y298-L372, F299-L372, S300-L372, A301-L372, E302-L372, G303-L372, F304-L372, R305-L372, N306-L372, T307-L372, L308-L372, R309-L372, G310-L372, L311-L372, G312-L372, T313-L372, P314-L372, H315-L372, R316-L372, A317-L372, R318-L372, T319-L372, S320-L372, A321-L372, T322-L372, N323-L372, G324-L372, T325-L372, R326-L372, A327-L372, A328-L372, L329-L372, A330-L372, Q331-L372, S332-L372, E333-L372, R334-L372, S335-L372, A336-L372, V337-L372, T338-L372, T339-L372, D340-L372, A341-L372, T342-L372, R343-L372, P344-L372, D345-L372, A346-L372, A347-L372, S348-L372, Q349-L372, G350-L372, L351-L372, L352-L372, R353-L372, P354-L372, S355-L372, D356-L372, S357-L372, H358-L372, S359-L372, L360-L372, S361-L372, S362-L372, F363-L372, T364-L372, Q365-L372, and/or C366-L372 of SEQ ID NO:2. Polynucleotide sequences encoding these polypeptides are also included in SEQ ID NO: 1. The present invention also encompasses the use of these N-terminal HGPRBMY3 deletion polypeptides as immunogenic and/or antigenic epitopes as described elsewhere herein.

In preferred embodiments, the following C-terminal HGPRBMY3 deletion polypeptides are encompassed by the present invention (of SEQ ID NO:2): M1-L372, M1-A371, M1-S370, M1-D369, M1-Q368, M1-P367, M1-C366, M1-Q365, M1-T364, M1-F363, M1-S362, M1-S361, M1-L360, M1-S359, M1-H358, M1-S357, M1-D356, M1-S355, M1-P354, M1-R353, M1-L352, M1-L351, M1-G350, M1-Q349, M1-S348, M1-A347, M1-A346, M1-D345, M1-P344, M1-R343, M1-T342, M1-A341, M1-D340, M1-T339, M1-T338, M1-V337, M1-A336, M1-S335, M1-R334, M1-E333, M1-S332, M1-Q331, M1-A330, M1-L329, M1-A328, M1-A327, M1-R326, M1-T325, M1-G324, M1-N323, M1-T322, M1-A321, M1-S320, M1-T319, M1-R318, M1-A317, M1-R316, M1-H315, M1-P314, M1-T313, M1-G312, M1-L311, M1-G310, M1-R309, M1-L308, M1-T307, M1-N306, M1-R305, M1-F304, M1-G303, M1-E302, M1-A301, M1-S300, M1-F299, M1-Y298, M1-Y297, M1-V296, M1-L295, M1-P294, M1-D293, M1-L292, M1-V291, M1-C290, M1-N289, M1-A288, M1-G287, M1-A286, M1-L285, M1-L284, M1-V283, M1-M282, M1-V281, M1-M280, M1-L279, M1-V278, M1-G277, M1-R276, M1-V275, M1-R274, M1-D273, M1-R272, M1-A271, M1-P270, M1-V269, M1-S268, M1-A267, M1-A266, M1-V265, M1-L264, M1-K263, M1-S262, M1-R261, M1-L260, M1-L259, M1-G258, M1-Y257, M1-V256, M1-A255, M1-L254, M1-T253, M1-S252, M1-N251, M1-Y250, M1-P249, M1-V248, M1-F247, M1-C246, M1-L245, M1-L244, M1-F243, M1-1242, M1-V241, M1-L240, M1-N239, M1-A238, M1-L237, M1-L236, M1-L235, M1-R234, M1-V233, M1-T232, M1-K231, M1-R230, M1-R229, M1-R228, M1-Q227, M1-S226, M1-Q225, M1-T224, M1-A223, M1-D222, M1-P221, M1-R220, M1-A219, M1-L218, M1-T217, M1-W216, M1-F215, M1-V214, M1-R213, M1-G212, M1-S211, M1-S210, M1-Y209, M1-V208, M1-V207, M1-A206, M1-A205, M1-L204, M1-P203, M1-L202, M1-L201, M1-F200, MI-G199, M1-L198, M1-A197, M1-E196, M1-A195, M1-L194, M1-L193, M1-V192, Ml-L191, Ml-P190, M1-L189, M1-L188, M1-R187, M1-G186, M1-K185, M1-W184, M1-L183, M1-E182, M1-D181, M1-S180, M1-F179, M1-S178, M1-E177, M1-F176, M1-C175, M1-L174, M1-R173, M1-V172, M1-E171, M1-L170, M1-D169, M1-R168, M1-Y167, M1-R166, M1-C165, M1-R164, M1-S163, M1-P162, M1-R161, M1-H160, M1-V159, M1-R158, M1-A157, M1-A156, M1-P155, M1-V154, M1-A153, M1-F152, M1-V151, M1-L150, M1-I149, M1-L148, M1-A147, M1-W146, M1-V145, M1-G144, M1-L143, M1-C142, M1-L141, M1-L140, M1-R139, M1-A138, M1-V137, M1-R136, M1-P135, M1-R134, M1-R133, M1-L132, M1-H131, M1-R130, M1-L129, M1-R128, M1-L127, M1-P126, M1-H125, M1-V124, M1-I123, M1-A122, M1-A121, M1-Y120, M1-R119, M1-D118, M1-V117, M1-N116, M1-115, M1-L114, M1-M113, M1-L112, M1-F111, M1-I110, M1-C109, M1-S108, M1-G107, M1-Y106, M1-M105, M1-N104, M1-M103, M1-Q102, M1-F101, M1-I100, M1-A99, M1-G98, M1-T97, M1-T96, M1-Q95, M1-C94, M1-L93, M1-L92, M1-D91, M1-P90, M1-F89, M1-P88, M1-W87, M1-H86, M1-H85, M1-L84, M1-A83, M1-Y82, M1-Y81, M1-S80, M1-L79, M1-R78, M1-V77, M1-P76, M1-L75, M1-S74, M1-L73, M1-T72, M1-F71, M1-L70, M1-L69, M1-D68, M1-S67, M1-A66, M1-A65, M1-L64, M1-N63, M1-C62, M1-M61, M1-Y60, M1-V59, M1-S58, M1-V57, M1-V56, M1-S55, M1-H54, M1-V53, M1-R52, M1-L51, M1-A50, M1-R49, M1-L48, M1-F47, M1-V46, M1-W45, M1-L44, M1-A43, M1-L42, M1-A41, M1-N40, M1-L39, M1-P38, M1-L37, M1-G36, M1-A35, M1-A34, M1-L33, M1-V32, M1-L31, M1-S30, M1-Y29, M1-V28, M1-V27, M1-L26, M1-H25, M1-L24, M1-R23, M1-H22, M1-T21, M1-P20, M1-R19, M1-Y18, M1-D17, M1-P16, M1-C15, M1-P14, M1-L13, M1-V12, M1-S11, M1-S10, M1-N9, M1-T8, and/or M1-S7 of SEQ ID NO:2. Polynucleotide sequences encoding these polypeptides are also included in SEQ ID NO: 1. The present invention also encompasses the use of these C-terminal HGPRBMY3 deletion polypeptides as immunogenic and/or antigenic epitopes as described elsewhere herein.

Alternatively, preferred polypeptides of the present invention may comprise polypeptide sequences corresponding to, for example, internal regions of the HGPRBMY3 polypeptide (e.g., any combination of both N- and C-terminal HGPRBMY3 polypeptide deletions) of SEQ ID NO:2. For example, internal regions could be defined by the equation: amino acid NX to amino acid CX, wherein NX refers to any N-terminal deletion polypeptide amino acid of HGPRBMY3 (SEQ ID NO:2), and where CX refers to any C-terminal deletion polypeptide amino acid of HGPRBMY3 (SEQ ID NO:2). Polynucleotides encoding these polypeptides are also included in SEQ ID NO: 1. The present invention also encompasses the use of these polypeptides as an immunogenic and/or antigenic epitope as described elsewhere herein.

### Example 11

### Method of Enhancing the Biological Activity or Functional Characteristics through Molecular Evolution

Although many of the most biologically active proteins known are highly effective for their specified function in an organism, they often possess characteristics that make them undesirable for transgenic, therapeutic, pharmaceutical, and/or industrial applications. Among these traits, a short physiological half-life is the most prominent problem, and is present either at the level of the protein, or the level of the proteins mRNA. The ability to extend the half-life, for example, is particularly important for using proteins in gene therapy, transgenic animal production, the bioprocess production and purification of the protein, and as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered G-protein coupled receptor may be constitutively active upon binding of its cognate ligand. Alternatively, an engineered G-protein coupled receptor may be constitutively active in the absence of ligand binding. In yet another example, an engineered GPCR may be capable of being activated with less than all of the regulatory factors and/or conditions typically required for GPCR activation (e.g., ligand binding, phosphorylation, conformational changes, etc.). Such GPCRs would be useful in screens to identify GPCR modulators, among other uses described herein.

Directed evolution is comprised of several steps. The first step is to establish a library of variants for the gene or protein of interest. The most important step is to then select for those variants that entail the activity for identification. The design of the screen is essential since the screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as descibed by Derbyshire, K. M. et al, Gene, 46:145-152, (1986), and Hill, DE, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (WPC, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, a randomly digested pool of small fragments of the gene of interest, created by Dnase I digestion, is introduced into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments-further diversifying the potential hybridation sites during the annealing step of the reaction.

A variety of reaction conditions could be utilized to carry-out the DNA shuffling reaction. However, specific reaction conditions for DNA shuffling are provided, for example, in PNAS, 91:10747, (1994).

Briefly, prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50mM Tris-HCL, pH 7.4/1 mM MgCl₂ for 10-20 min. at room temperature. The resulting fragments of 10-50 bp could then be purified by running them through a 2% low-melting point agarose gel by electrophoresis onto DE81 ion-exchange paper (Whatman) or could be purified using Microcon concentrators (Amicon) of the appropriate molecular weight cuttoff, or could use oligonucleotide purification columns (Qiagen), in addition to other methods known in the art. If using DE81 ion-exchange paper, the 10-50 bp fragments could be eluted from said paper using 1M NaCl, followed by ethanol precipitation.

The resulting purified fragments would then be subjected to a PCR assembly reaction by re-suspension in a PCR mixture containing: 2 mM of each DNTP, 2.2 mM MgCl₂, 50 mM KCl, 10 mM Tris*HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C for 60s; 94 C for 30s, 50-55 C for 30s, and 72 C for 30s using 30-45 cycles, followed by 72 C for 5 min using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8um of each primer and subjecting this mixture to 15 cycles of PCR (using 94 C for 30s, 50 C for 30s, and 72 C for 30s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailered to the desired level of mutagenesis using the methods described by Zhao, et al. (Nucl Acid Res., 25(6):1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16,000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host. For example, a particular varient of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native strucuture which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel varient that provided the desired characteristics.

Likewise, the invention encompasses the application of DNA shuffling technology to the evolution of polynucletotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homolog sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

The contents of all patents, patent applications, published PCT applications and articles, books, references, reference manuals and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

References

1. Rees, S., Brown, S., Stables, J.: “Reporter gene systems for the study of G Protein Coupled Receptor signalling in mammalian cells”. In Milligan G. (ed.): Signal Transduction: A practical approach. Oxford: Oxford University Press, 1999: 171-221.

2. Alam, J., Cook, J. L.: “Reporter Genes: Application to the study of mammalian gene transcription”. Anal. Biochem. 1990; 188: 245-254.

3. Selbie, L. A. and Hill, S. J.: “G protein-coupled receptor cross-talk: The fine-tuning of multiple receptor-signaling pathways”. TiPs. 1998; 19: 87-93.

4. Boss, V., Talpade, D. J., and Murphy, T. J.: “Induction of NFAT mediated transcription by Gq-coupled Receptors in lympoid and non-lymphoid cells”. JBC. 1996; 271: 10429-10432.

5. George, S. E., Bungay, B. J., and Naylor, L. H.: “Functional coupling of endogenous serotonin (5-HT1B) and calcitonin (C1a) receptors in CHO cells to a cyclic AMP-responsive luciferase reporter gene”. J. Neurochem. 1997; 69: 1278-1285.

6. Suto, C M, Igna D M: “Selection of an optimal reporter for cell-based high throughput screening assays”. J. Biomol. Screening. 1997; 2: 7-12.

7. Zlokarnik, G., Negulescu, P. A., Knapp, T. E., More, L., Burres, N., Feng. L., Whitney, M., Roemer, K. and Tsien, R. Y. “Quantitation of transcription and clonal selection of single living cells with a B-Lactamase Reporter”. Science. 1998; 279: 84-88.

8. S. Fiering et. al., Genes Dev. 4, 1823 (1990).

9. J. Karttunen and N.Shastri, PNAS 88, 3972(1991).

10. Hawes, B. E., Luttrell. L. M., van Biesen, T., and Lefkowitz, R. J. (1996) JBC 271, 12133-12136.

11. Gilman, A. G. (1987) Annul. Rev. Biochem. 56,615-649.

12. Maniatis et al., Cold Spring Harbor Press, 1989.

13. Salcedo, R., Ponce, M. L., Young, H. A., Wasserman, K., Ward, J. M., Kleinman, H. K., Oppenheim, J J., Murphy, W. J “Human endothelial cells express CCF2 and respond to MCP-1: direct role of MCP-1 in angiogepesis and tumor progression”. Blood. 2000; 96 (1): 34-40.

14. Sica, A., Saccani, A., Bottazzi, B., Bernasconi, S., Allavena. P., Gaetano, B., LaRossa, G., Scotton, C., Balkwill F., Mantovani, A. “Defective expression of the monocyte chemotactic protein I receptor CCF2 in macrophages associated with human ovarian carcinoma”. J. Immunology. 2000; 164: 733-8.

15. Kypson, A., Hendrickson, S., Aklter, S., Wilson, K., McDonald, P., Lilly, R., Dolber, P., Glower, D., Lefkowitz, R., Koch, W. “Adenovirus-mediated gene transfer of the B2 AR to donor hearts enhances cardiac function”. Gene Therapy. 1999; 6: 1298-304.

16. Dorn, G. W., Tepe, N. M., Lorenz, J. N., Kock, W. J., Ligget S. B. “Low and high level transgenic expression of B2AR differentially affect cardiac hypertrophy and function in Galpha q-overexpressing mice”. PNAS. 1999; 96: 6400-5.

17. J. Wess. “G protein coupled receptor: molecular mechanisms involved in receptor activation and selectivity of G-protein recognition”. FASEB. 1997; 11:346-354.

18. Whitney, M, Rockenstein, E, Cantin, G., Knapp, T., Zlokamik, G., Sanders, P., Durick, K., Craig, F. F., and Negulescu, P. A. “A genome-wide functional assay of signal transduction in living mammalian cells”. Nature Biotech. 1998; 16: 1329-1333.

19. BD Biosciences: FACS Vantage SE Training Manual. Part Number 11-11020-00 Rev. A. August 1999.

20. Chen, G., Jaywickreme, C., Way, J., Armour S., Queen K., Watson., C., Ignar, D., Chen, W. J., Kenakin, T. “Constitutive Receptor systems for drug discovery”. J. Pharmacol. Toxicol. Methods 1999; 42: 199-206.

21. Blahos, J., Fischer, T., Brabet, I., Stauffer, D., Rovelli, G., Bockaert, J., and Pin, J. -P. “A novel Site on the G alpha-protein that Rocognized Heptahelical Receptors”. J.Biol. Chem. 2001; 275, No. 5, 3262-69.

22. Offermanns, S. & Simon, M. I. “G alpha 15 and G alpha 16 Couple a Wide Variety of Receptors to Phospholipase C”. J. Biol. Chem. 1995; 270, No. 25, 15175-80.

What is claimed is:

1. An isolated nucleic acid molecule consisting of a polynucleotide having a nucleotide sequence selected from the group consisting of:
a) a polynucleotide fragment of SEQ ID NO: 1 or a polynucleotide fragment of the cDNA sequence included in ATCC Deposit No:PTA-2681, which is hybridizable to SEQ ID NO:1;
b) a polynucleotide encoding a polypeptide fragment of SEQ ID NO:2 or a polypeptide fragment encoded by the cDNA sequence included in ATCC Deposit No:PTA-2681, which is hybridizable to SEQ ID NO: 1;
c) a polynucleotide encoding a polypeptide domain of SEQ ID NO:2 or a polypeptide domain encoded by the cDNA sequence included in ATCC Deposit No:PTA-2681, which is hybridizable to SEQ ID NO1;
d) a polynucleotide encoding a polypeptide epitope of SEQ ID NO:2 or a polypeptide epitope encoded by the cDNA sequence included in ATCC Deposit No:PTA-2681, which is hybridizable to SEQ ID NO:1;
e) a polynucleotide encoding a polypeptide of SEQ ID NO:2 or the cDNA sequence included in ATCC Deposit No:PTA-2681, which is hybridizable to SEQ ID NO:1, having biological activity;
f) a polynucleotide which is a variant of SEQ ID NO: 1;
g) a polynucleotide which is an allelic variant of SEQ ID NO: 1;
h) a polynucleotide which encodes a species homologue of the SEQ ID NO:2;
i) a polynucleotide which represents the complimentary sequence (antisense) of SEQ ID NO: 1;
j) a polynucleotide corresponding to nucleotides 4 to 1116 of SEQ ID NO:1;
k) a polynucleotide corresponding to nucleotides 1 to 1116 of SEQ ID NO:1; or
l) a polynucleotide capable of hybridizing under stringent conditions to any one of the polynucleotides specified in (a)-(k),
wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a G-protein coupled receptor protein.

3. The isolated nucleic acid molecule of claim 1, wherein the polynucleotide fragment comprises a nucleotide sequence encoding the sequence identified as SEQ ID NO:2 or the polypeptide encoded by the cDNA sequence included in ATCC Deposit No:PTA-2681, which is hybridizable to SEQ ID NO:1.

4. The isolated nucleic acid molecule of claim 1, wherein the polynucleotide fragment comprises the entire nucleotide sequence of SEQ ID NO: 1 or the cDNA sequence included in ATCC Deposit No:PTA-2681, which is hybridizable to SEQ ID NO:1.

5. The isolated nucleic acid molecule of claim 2, wherein the nucleotide sequence comprises sequential nucleotide deletions from either the C-terminus or the N-terminus.

6. The isolated nucleic acid molecule of claim 3, wherein the nucleotide sequence comprises sequential nucleotide deletions from either the C-terminus or the N-terminus.

7. A recombinant vector comprising the isolated nucleic acid molecule of claim 1.

8. A method of making a recombinant host cell comprising the isolated nucleic acid molecule of claim 1.

9. A recombinant host cell produced by the method of claim 8.

10. The recombinant host cell of claim 9 comprising vector sequences.

11. An isolated polypeptide comprising an amino acid sequence at least 95% identical to a sequence selected from the group consisting of:
a ) a polypeptide fragment of SEQ ID NO:2 or the encoded sequence included in ATCC Deposit No:PTA-2681;
b ) a polypeptide fragment of SEQ ID NO:2 or the encoded sequence included in ATCC Deposit No:PTA-2681, having biological activity;
c ) a polypeptide domain of SEQ ID NO:2 or the encoded sequence included in ATCC Deposit No:PTA-2681;
d ) a polypeptide epitope of SEQ ID NO:2 or the encoded sequence included in ATCC Deposit No:PTA-2681;
e) a full length protein of SEQ ID NO:2 or the encoded sequence included in ATCC Deposit No:PTA-2681;
f) a variant of SEQ ID NO:2;
g) an allelic variant of SEQ ID NO:2;
h) a species homologue of SEQ ID NO:2; or
i) a polypeptide corresponding to amino acids 2 to 372 of SEQ ID NO:2.

12. The isolated polypeptide of claim 11, wherein the full length protein comprises sequential amino acid deletions from either the C-terminus or the N-terminus.

13. An isolated antibody that binds specifically to the isolated polypeptide of claim 11.

14. A recombinant host cell that expresses the isolated polypeptide of claim 11.

15. A method of making an isolated polypeptide comprising:
a ) culturing the recombinant host cell of claim 14 under conditions such that said polypeptide is expressed; and
b ) recovering said polypeptide.

16. A polypeptide produced by claim 15.

17. A method for preventing, treating, or ameliorating a medical condition, comprising administering to a mammalian subject a therapeutically effective amount of the polypeptide of claim 11 or the polynucleotide of claim 1.

18. A method of diagnosing a pathological condition or a susceptibility to a pathological condition in a subject comprising:
a ) determining the presence or absence of a mutation in the polynucleotide of claim 1; and
b ) diagnosing a pathological condition or a susceptibility to a pathological condition based on the presence or absence of said mutation.

19. A method of diagnosing a pathological condition or a susceptibility to a pathological condition in a subject comprising:
a) determining the presence or amount of expression of the polypeptide of claim 11 in a biological sample; and
b) diagnosing a pathological condition or a susceptibility to a pathological condition based on the presence or amount of expression of the polypeptide.

20. A gene corresponding to the cDNA sequence of SEQ ID NO:2.

21. A method of identifying an activity in a biological assay, wherein the method comprises:
a) expressing the HGPRBMY3 sequence as set forth in SEQ ID NO:2 in a host cell having; and
b ) measuring the resulting activity of the expressed HGPRBMY3.

22. A method for identifying a binding partner to the polypeptide of claim 11 comprising:
a ) contacting the polypeptide of claim 11 with a binding partner; and
b ) determining whether the binding partner effects an activity of the polypeptide.

23. A method of identifying a compound that modulates the biological activity of HGPRBMY3, or a GPCR, comprising:
a) combining a candidate modulator compound with a host cell containing a vector according to claim 7, wherein HGPRBMY3 is expressed by the cell; and
b) measuring an effect of the candidate modulator compound on the activity of the expressed HGPRBMY3.

24. A compound that modulates the biological activity of human HGPRBMY3 as identified by the method according to claim 21, 22, or 23.

25. The method of claim 22 wherein said binding partner is a peptide.

26. A method of treating a disease, disorder, or condition related to the colon, breast, ovaries, or immune system, comprising administering the G-protein coupled receptor polypeptide or homologue according to claim 11 in an amount effective to treat the colon-, testes-, breast-, ovary-, or immune-related disorder.

27. The polynucleotide of claim 2, further comprising a polynucleotide localized in colon, breast, ovarian, or testes cell lines.

28. The polypeptide of claim 11, further comprising a polypeptide expressed in colon, breast, ovarian, or testes cell lines.

29. A cell comprising NFAT/CRE and the polypeptide of claim 11.

30. A cell comprising NFAT G alpha 15 and the polypeptide of claim 11.

31. A method of screening for candidate compounds capable of modulating activity of a G-protein coupled receptor-encoding polypeptide, comprising:
a) contacting a test compound with the cell of claim 29 or 30; and
b) selecting as candidate modulating compounds those test compounds that modulate activity of the G-protein coupled receptor polypeptide.

32. The method according to claim 31, wherein the candidate compounds are agonists or antagonists of G-protein coupled receptor activity.

33. The method according to claim 32, wherein the candidate compounds are peptides.

34. The method according to claim 32, wherein the polypeptide activity is associated with the colon, testes, breast, ovaries, or immune system.
